(19) United States
(12) Patent Application Publication
(10) Pub. No.: US 2018/0284120 A1
(43) Pub. Date: Oct. 4, 2018

(54) METHODS FOR DETERMINING A BREAST CANCER-ASSOCIATED DISEASE STATE AND ARRAYS FOR USE IN THE METHODS

(71) Applicant: IMMUNOVIA AB, Lund (SE)

(72) Inventors: Carl Arne Krister Borrebaeck, Lund (SE); Christer Lars Bertil Wingren, Sandby (SE)

(21) Appl. No.: 15/855,156

(22) Filed: Dec. 27, 2017

Related U.S. Application Data
(63) Continuation of application No. 14/391,144, filed on Oct. 7, 2014, now abandoned.

(30) Foreign Application Priority Data
Apr. 10, 2012 (GB) 1206323.6

(51) Int. Cl. G01N 33/574; C12Q 1/6886

(52) U.S. Cl. CPC G01N 33/57415; G01N 2570/00; G01N 2446/00; C12Q 2600/158; C12Q 1/6886

(57) ABSTRACT

The present invention provides a method for determining a breast cancer-associated disease state comprising the steps of: a) providing a sample to be tested; and b) determining a biomarker signature of the test sample by measuring the presence and/or amount in the test sample of one or more biomarker selected from the group defined in Table 1; wherein the presence and/or amount in the test sample of the one or more biomarker selected from the group defined in Table 1 is indicative of the breast cancer-associated disease state. The invention further provides arrays and kits fir use in the same.

This application a continuation application of U.S. patent application Ser. No. 14/391,144, filed Oct. 7, 2014, which is a § 371 application of PCT/IB2013/052858, filed Apr. 10, 2013, which in turn claims priority to GB Application 1206323.6, filed Apr. 10, 2012. The entire disclosure of each of the foregoing applications is incorporated by reference herein.

### FIELD OF THE INVENTION

The present invention provides methods for determining a breast cancer-associated disease state, as well as arrays and kits for use in such methods.

### BACKGROUND OF THE INVENTION

Breast cancer is the most frequently diagnosed cancer and the leading cause of cancer death among women, accounting for 23% of the total cancer cases and 14% of the cancer related deaths (Jemal et al., 2011). Traditional clinic pathological parameters, such as histological grading, tumor size, age, lymph node involvement, and hormonal receptor status are used to decide treatment and estimate prognosis (Ciocca and Elledge, 2000; Elston and Ellis, 1991; Hondermarck et al., 2008; Hudis, 2007; Slamon et al., 2001). Histological grading, one of the most commonly used prognostic factors, is a combined score, based on microscopic evaluation of morphological and cytological features of tumor cells, reflecting the aggressiveness of a tumor. This combined score is then used to stratify breast cancer tumors into; grade 1—slow growing and well differentiated, grade 2—moderately differentiated, and grade 3—highly proliferative and poorly differentiated (Elston and Ellis, 1991). However, the clinical value of histologic grade for patient prognosis has been questioned, mainly reflecting the current challenges associated with grading the tumors (Frierson et al., 1995; Robbins et al., 1995). Furthermore, 30-60% of the tumors are classified as histologic grade 2, which has turned out to represent a very heterogeneous patient cohort and proven to be less informative for clinical decision making (Sotiriou et al., 2006). Clearly, traditional clinical laboratory parameters are still not sufficient for adequate prognosis and risk-group discrimination, and for predicting whether a given treatment will be successful. As a result, some patients will be over-treated, under-treated, or even treated with a therapy that will not offer any benefit. Hence, a deeper molecular understanding of breast cancer biology and tumor progression, in combination with improved ways to individualize prognosis and treatment decisions are required in order to further advance prognostic and, consequently, therapeutic outcomes (Dowsett et al., 2007).

### DISCLOSURE OF THE INVENTION

To date, a set of genomic efforts have generated molecular signatures for subgrouping of breast cancer types (Ivshina et al., 2006; Perou et al., 2000; Sorlie et al., 2001) as well as for breast cancer prognostics and risk stratification (Paik et al., 2004; van 't Veer et al., 2002; van de Vijver et al., 2002). On the other hand, proteomic findings have been anticipated to accelerate the translation of key discoveries into clinical practice (Hanash, 2003). In this context, classical mass spectrometry (MS)-based proteomics have generated valuable inventories of breast cancer proteomes, targeting mainly cell lines and few tissue samples (Bouchal et al., 2009; Geiger et al., 2010; Geiger et al., 2012; Gong et al., 2008; Kang et al., 2010; Strande et al., 2009; Sutton et al., 2010), and more recently, affinity proteomics efforts delivered the first multiplexed serum portraits for breast cancer diagnosis and for predicting the risk of relapse (Carlsson et al., 2008; Carlsson et al., 2011). But despite the recent technical advancements, generating detailed protein expression profiles of large cohorts of crude proteomes, e.g. tissue extracts, in a sensitive and reproducible manner remains a challenge using either classical proteomic technologies (Aebersold and Mann, 2003) or affinity proteomics (Borrebaeck and Wingren, 2011).

To resolve these issues, we have recently developed the global proteome survey (GPS) technology platform (Wingren et al., 2009), combining the best features of affinity proteomics and MS. GPS is suited for discovery endeavours, reproducibly deciphering crude proteomes in a sensitive and quantitative manner (Olsson et al., 2012; Olsson et al., 2011).

In this study, we delineated in-depth molecular tissue portraits of histologic graded breast cancer tissues reflecting tumour progression using GPS. To this end, 52 breast cancer tissue proteomes were profiled, to the best of our knowledge, representing one of the largest label-free LC-MS/MS-based breast cancer tissue studies. The protein expression profiles were successfully validated using an orthogonal method. In the long-term run, these tissue biomarker portraits could pave the way for improved classification and prognosis.

Accordingly, a first aspect of the invention provides a method for determining a breast cancer-associated disease state comprising the steps of:
a) providing a sample to be tested; and
b) determining a biomarker signature of the test sample by measuring the presence and/or amount in the test sample of one or more biomarker selected from the group defined in Table 1;
   wherein the presence and/or amount in the test sample of the one or more biomarker selected from the group defined in Table 1 is indicative of the breast cancer-associated disease state. Hence, in effect, steps (b) comprises an additional step of step ((b)(i)) of determining a breast cancer associated disease state using or based on the presence and/or amount in the test sample of the one or more biomarker selected from the group defined in Table 1.

By “breast cancer-associated disease state” we mean the histological grade of breast cancer cells and/or the metastasis-free survival time of an individual comprising breast cancer cells.

The breast cancer-associated disease state may be the histological grade (of breast cancer cells) and/or the metastasis-free survival time (of an individual).

By “biomarker” we mean a naturally-occurring biological molecule, or component or fragment thereof, the measurement of which can provide information useful in the prognosis of breast cancer. For example, the biomarker may be a naturally-occurring protein or carbohydrate moiety, or an antigenic component or fragment thereof.

Preferably the sample to be tested is provided from a mammal. The mammal may be any domestic or farm animal. Preferably, the mammal is a rat, mouse, guinea pig, cat, dog, horse or a primate. Most preferably, the mammal is human. Preferably the sample is a cell or tissue sample (or derivative thereof) comprising or consisting of breast cancer cells or equally preferred, protein or nucleic acid derived from a cell or tissue sample comprising or consisting of breast cancer cells. Preferably test and control samples are derived from the same species.

Where the breast cancer-associated disease state is or comprises the histological grade of breast cancer cells, the method may further comprise the steps of:
c) providing one or more control sample comprising or consisting of histological grade 1 breast cancer cells, histological grade 2 breast cancer cells and/or histological grade 3 breast cancer cells; and
d) determining a biomarker signature of the control sample(s) by measuring the presence and/or amount in the control sample(s) of the one or more biomarker measured in step (b);
   wherein the presence of breast cancer cells is identified in the event that the presence and/or amount in the test sample of the one or more biomarker measured in step (b):
i) corresponds to the presence and/or amount in a control sample comprising or consisting breast cancer cells of a first histological grade (where present);
ii) is different to the presence and/or amount in a control sample comprising or consisting breast cancer cells of a second histological grade (where present); and/or
iii) is different to the presence and/or amount in a control sample comprising or consisting breast cancer cells of a third histological grade (where present).

Hence, if the first histological grade was Elston grade 1, the second and third histological grades (where present) would be Elston grade 2 and Elston Grade 3 (or vice versa). Where the first histological grade was Elston grade 2, the second and third histological grades (where present) would be Elston grade 1 and Elston Grade 3 (or vice versa). Where the first histological grade was Elston grade 3, the second and third histological grades (where present) would be Elston grade 1 and Elston Grade 2 (or vice versa).

By “corresponds to the presence and/or amount in a control sample comprising or consisting breast cancer cells of a first histological grade” we mean the presence and or amount is identical to that of a control sample comprising or consisting of breast cancer cells of a first histological grade; or closer to that of a control sample comprising or consisting breast cancer cells of a first histological grade than to a control sample comprising or consisting breast cancer cells of a second histological grade and/or a control sample comprising or consisting breast cancer cells of a third histological grade (or to predefined reference values representing the same). Preferably the presence and/or amount is at least 60% of that of the control sample comprising or consisting breast cancer cells of a first histological grade, for example, at least 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100%.

By “is different to the presence and/or amount in a control sample comprising or consisting breast cancer cells of a third histological grade” we mean the presence and or amount differs from that of the control sample comprising or consisting breast cancer cells of a first histological grade or than that of a control sample comprising or consisting breast cancer cells of a second histological grade and/or a control sample comprising or consisting breast cancer cells of a third histological grade (or to predefined reference values representing the same). Preferably the presence and/or amount is no more than 40% of that of the control sample comprising or consisting breast cancer cells of a second histological grade, and/or the control sample comprising or consisting breast cancer cells of a third histological grade for example, no more than 39%, 38%, 37%, 36%, 35%, 34%, 33%, 32%, 31%, 30%, 29%, 28%, 27%, 26%, 25%, 24%, 23%, 22%, 21%, 20%, 19%, 18%, 17%, 16%, 15%, 14%, 13%, 12%, 11%, 10%, 9%, 8%, 7%, 6%, 5%, 4%, 3%, 2%, 1% or 0%.

Preferably histological grade control sample comprises or consists of a single histological grade of breast cancer cells. Preferably, step (c) comprises or consists of:
i) providing one or more control sample comprising or consisting of histological grade 1 breast cancer cells; providing one or more control sample comprising or consisting of histological grade 2 breast cancer cells; and providing one or more control sample comprising or consisting of histological grade 3 breast cancer cells;
ii) providing one or more control sample comprising or consisting of histological grade 1 breast cancer cells; and providing one or more control sample comprising or consisting of histological grade 2 breast cancer cells;
iii) providing one or more control sample comprising or consisting of histological grade 1 breast cancer cells; and providing one or more control sample comprising or consisting of histological grade 3 breast cancer cells;
iv) providing one or more control sample comprising or consisting of histological grade 2 breast cancer cells; and providing one or more control sample comprising or consisting of histological grade 3 breast cancer cells;
v) providing one or more control sample comprising or consisting of histological grade 1 breast cancer cells;
vi) providing one or more control sample comprising or consisting of histological grade 2 breast cancer cells; or
vii) providing one or more control sample comprising or consisting of histological grade 3 breast cancer cells.

Where the breast cancer-associated disease state is or comprises the metastasis-free survival time of an individual the method may further comprise the steps of:
c) providing one or more first control sample comprising or consisting of breast cancer cells from an individual with less than 10 years metastasis-free survival; and/or one or more second control sample comprising or consisting of breast cancer cells from an individual with 10 or more years metastasis-free survival; and
d) determining a biomarker signature of the control sample(s) by measuring the presence and/or amount in the control sample(s) of the one or more biomarker measured in step (b);
wherein the metastasis-free survival time of an individual is identified as less than 10 years in the event that the presence and/or amount of the one or more biomarker measured in step (b) corresponds to the presence and/or amount of the first control sample (where present) and/or is different to the presence and/or amount of the second control sample (where present);
and wherein the metastasis-free survival time of an individual is identified as more than 10 years in the event that the presence and/or amount of the one or more biomarker measured in step (b) is different to the presence and/or amount of the first control sample (where present) and/or corresponds to the presence and/or amount of the second control sample (where present)

By “corresponds to the presence and/or amount of the one or more first control sample” we mean the presence and or amount is identical to that of the one or more first control sample; or closer to that of a first control sample than to the one or more second control sample (or to predefined reference values representing the same). Preferably the presence and/or amount is at least 60% of that of the first control sample, for example, at least 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100%.

By “is different to the presence and/or amount of the one or more a second control sample” we mean the presence and or amount differs from that of the second control sample (or to predefined reference values representing the same). Preferably the presence and/or amount is no more than 40% of that of the second control sample, for example, no more than 39%, 38%, 37%, 36%, 35%, 34%, 33%, 32%, 31%, 30%, 29%, 28%, 27%, 26%, 25%, 24%, 23%, 22%, 21%, 20%, 19%, 18%, 17%, 16%, 15%, 14%, 13%, 12%, 11%, 10%, 9%, 8%, 7%, 6%, 5%, 4%, 3%, 2%, 1% or 0%.

Preferably, the one or more first and/or second metastasis-free survival time control sample is of the same histological grade as the sample to be tested.

Preferably, the one or more control samples are age- and/or sex-matched for the individual to be tested. In other words, the healthy individual is approximately the same age (e.g. within 5 years) and is the same sex as the individual to be tested.

Preferably, the presence and/or amount in the test sample of the one or more biomarkers measured in step (b) are compared against predetermined reference values.

Hence, it is preferred that the presence and/or amount in the test sample of the one or more biomarker measured in step (b) is significantly different (i.e. statistically different) from the presence and/or amount of the one or more biomarker measured in step (d) or the predetermined reference values. For example, as discussed in the accompanying Examples, significant difference between the presence and/or amount of a particular biomarker in the test and control samples may be classified as those where p<0.05 (for example, where p<0.04, p<0.03, p<0.02 or where p<0.01).

Hence, the method of the first aspect of the invention may comprise or consist of determining the histological grade of breast cancer cells and the metastasis-free survival time of an individual (either concurrently or consecutively).

Preferably step (b) comprises or consists of measuring the presence and/or amount in the test sample of one or more biomarkers selected from the group defined in Table 1, for example at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78 or at least 79 biomarkers selected from the group defined in Table 1.

Hence, the first aspect of the invention may comprise or consist of a method for determining the histological grade of breast cancer cells (i.e., staging of breast cancer samples to determine histological grade) comprising the steps of:
a) providing a sample to be tested;
b) determining a biomarker signature of the test sample by measuring the presence and/or amount in the test sample of one or more biomarker selected from the group defined in Table 1;
wherein the presence and/or amount in the test sample of the one or more biomarker selected from the group defined in Table 1 is indicative of the histological grade of the breast cancer cells.

By “determining the histological grade of breast cancer cells” we mean that the breast cancer cells of a sample are categorised as histological grade 1 (i.e., Elston grade 1), histological grade 2 (i.e., Elston grade 2) or histological grade 3 (i.e., Elston grade 3) as defined in Elston, C. W., and Ellis, I. O. (1991). Pathological prognostic factors in breast cancer. I. The value of histological grade in breast cancer: experience from a large study with long-term follow-up. Histopathology 19, 403-410 which is incorporated herein by reference.

Where the method comprises or consists of determining the histological grade of breast cancer cells, step (b) may comprise or consist of measuring the presence and/or amount in the test sample of one or more biomarkers selected from the group defined in Table 1A, for example at least 2, biomarkers selected from the group defined in Table 1A. Preferably step (b) comprises or consists of measuring the presence and/or amount in the test sample of one or more biomarkers selected from the group defined in Table 1B, for example at least 2, 3, 4, 5, 6, 7, 8, 9, 10, 11, 12, 13, 14, 15, 16, 17, 18, 19, 20, 21, 22, 23, 24, 25, 26, 27, 28, 29 or at least 30 biomarkers selected from the group defined in Table 1B. Preferably step (b) comprises or consists of measuring the presence and/or amount in the test sample of one or more biomarkers selected from the group defined in Table 10, for example at least 2, 3, 4, 5, 6, 7, 8, 9, 10, 11, 12, 13, 14, 15, 16, 17, 18, 19, 20, 21, 22, 23, 24, 25, 26, 27 or at least 28 biomarkers selected from the group defined in Table 10. Less preferably, step (b) comprises or consists of measuring the presence and/or amount in the test sample of one or more biomarkers selected from the group defined in Table 1D, for example at least 2, 3, 4, 5, 6, 7, 8, 9 or at least 10 biomarkers selected from the group defined in Table 1D. Also less preferably, step (b) comprises or consists of measuring the presence and/or amount in the test sample of one or more biomarkers selected from the group defined in Table 1E, for example at least 2, 3, 4, 5, 6, 7, 8 or at least 9 biomarkers selected from the group defined in Table 1E. Hence, step (b) may comprise or consist of measuring the presence and/or amount in the test sample of all of the biomarkers defined in Table 1.

Hence, the first aspect of the invention may comprise or consist of a method for determining the metastasis-free survival time of an individual comprising the steps of:
a) providing a sample to be tested;
b) determining a biomarker signature of the test sample by measuring the presence and/or amount in the test sample of one or more biomarker selected from the group defined in Table 1;
wherein the presence and/or amount in the test sample of the one or more biomarker selected from the group defined in Table 1 is indicative of the metastasis-free survival time of the individual.

By “determining the metastasis-free survival time of an individual” we mean that the individual from which the test sample is obtained is prognosed to have a metastasis-free survival time (distant metastasis-free survival/DMFS) of either less than 10 years or greater than 10 years from initial diagnosis.

Where the method comprises or consists of determining the metastasis-free survival time of an individual step (b) may comprise or consist of measuring the presence and/or amount in the test sample of one or more biomarkers selected from the group defined in Table 1A, for example at least 2, biomarkers selected from the group defined in Table 1A. Preferably step (b) comprises or consists of measuring the presence and/or amount in the test sample of one or more biomarkers selected from the group defined in Table 1B, for example at least 2, 3, 4, 5, 6, 7, 8, 9, 10, 11, 12, 13, 14, 15, 16, 17, 18, 19, 20, 21, 22, 23, 24, 25, 26, 27, 28, 29 or at least 30 biomarkers selected from the group defined in Table 1B. Preferably step (b) comprises or consists of measuring the presence and/or amount in the test sample of one or more biomarkers selected from the group defined in Table 1D, for example at least 2, 3, 4, 5, 6, 7, 8, 9 or at least 10 biomarkers selected from the group defined in Table 1D. Preferably step (b) comprises or consists of measuring the presence and/or amount in the test sample of all of the defined in Table 1A, Table 1B and Table 1D.

Where the method comprises or consists of determining the metastasis-free survival time of an individual, although less preferred, step (b) may comprise or consist of measuring the presence and/or amount in the test sample of one or more biomarkers selected from the group defined in Table 10, for example at least 2, 3, 4, 5, 6, 7, 8, 9, 10, 11, 12, 13, 14, 15, 16, 17, 18, 19, 20, 21, 22, 23, 24, 25, 26, 27 or at least 28 biomarkers selected from the group defined in Table 10. Also less preferably, step (b) may comprise or consist of measuring the presence and/or amount in the test sample of one or more biomarkers selected from the group defined in Table 1E, for example at least 2, 3, 4, 5, 6, 7, 8 or at least 9 biomarkers selected from the group defined in Table 1E. Also less preferably step (b) may comprise or consist of measuring the presence and/or amount in the test sample of all of the biomarkers defined in Table 10 and Table 1E.

Hence, although less preferred, the method of the first aspect of the invention may comprise or consist of determining the metastasis-free survival time of an individual wherein step (b) comprises or consists of measuring the presence and/or amount in the test sample of all of the biomarkers defined in Table 1.

Hence, the method according to the first aspect of the invention may include measuring SPON1 expression. The method may include measuring KERA expression. The method may include measuring APCS expression. The method may include measuring ATP6V1G1 expression. The method may include measuring RPS27L expression. The method may include measuring DPYSL3 expression. The method may include measuring ERP44 expression. The method may include measuring RAPGEF1 expression. The method may include measuring ACLY expression. The method may include measuring CMA1 expression. The method may include measuring MCM3 expression. The method may include measuring ANGPTL2 expression. The method may include measuring AEBP1 expression. The method may include measuring UBE2V2 expression. The method may include measuring MIS18BP1 expression. The method may include measuring CLCF1 expression. The method may include measuring ABAT expression. The method may include measuring SLC25A5 expression. The method may include measuring STIP1 expression. The method may include measuring OLFML3 expression. The method may include measuring CD3G expression. The method may include measuring MCM7 expression. The method may include measuring SLC25A11 expression. The method may include measuring NOP56 expression. The method may include measuring RRP8 expression. The method may include measuring SLTM expression. The method may include measuring TSN expression. The method may include measuring ECH1 expression. The method may include measuring PRELP expression. The method may include measuring SARS expression. The method may include measuring RPS25 expression. The method may include measuring ESYT1 expression. The method may include measuring PODN expression. The method may include measuring RPRD1B expression. The method may include measuring RPLPOP6 expression. The method may include measuring CD300LG expression. The method may include measuring SUGT1 expression. The method may include measuring POTEF expression. The method may include measuring KARS expression. The method may include measuring NDUFS2 expression. The method may include measuring HNRNPH2 expression. The method may include measuring CALU expression. The method may include measuring EIF3B expression. The method may include measuring SLC4A1AP expression. The method may include measuring RPS5 expression. The method may include measuring PLXDC2 expression. The method may include measuring KIAA1324 expression. The method may include measuring MRC1 expression. The method may include measuring RPRD1A expression. The method may include measuring SHMT2 expression. The method may include measuring CCT4 expression. The method may include measuring TSSC1 expression. The method may include measuring IKZF3 expression. The method may include measuring UBE2Q1 expression. The method may include measuring PSMD9 expression. The method may include measuring SNRNP70 expression. The method may include measuring RALB expression. The method may include measuring ACO2 expression. The method may include measuring MYO18A expression. The method may include measuring QARS expression. The method may include measuring PABPC4 expression. The method may include measuring SCGB1D2 expression. The method may include measuring PFKP expression. The method may include measuring SLC3A2 expression. The method may include measuring ASPN expression. The method may include measuring CD38 expression. The method may include measuring MXRAS expression. The method may include measuring CDK1 expression. The method may include measuring STC2 expression. The method may include measuring CTSC expression. The method may include measuring NOP58 expression. The method may include measuring PGK1 expression. The method may include measuring FKBP3 expression. The method may include measuring GSTM3 expression. The method may include measuring CALML5 expression. The method may include measuring PML expression. The method may include measuring ADAMTS4 expression. The method may include measuring THBS1 expression. The method may include measuring FN1 expression.

In one preferred embodiment, step (b) comprises or consists of measuring the presence and/or amount in the test sample of one or more biomarker selected from the group consisting of MCM7, NOP56, MCM3, PABPC4, MXRAS, STC2, SCGB1D2 and ANGPTL2. For example, step (b) may comprise or consist of measuring the presence and/or amount in the test sample of 2, 3, 4, 5, 6, 7 or 8 of these biomarkers. Preferably, in this embodiment, the breast cancer-associated disease state is histological grade; however, less preferably the breast cancer-associated disease state is or also includes metastasis-free survival time.

In another preferred embodiment, step (b) comprises or consists of measuring the presence and/or amount in the test sample of one or more biomarker selected from the group consisting of OLFML3, SPON1, PODN and ASPN. For example, step (b) may comprise or consist of measuring the presence and/or amount in the test sample of 2, 3 or 4 of these biomarkers. Preferably, in this embodiment, the breast cancer-associated disease state is histological grade; however, less preferably the breast cancer-associated disease state is or also includes metastasis-free survival time.

By “expression” we mean the level or amount of a gene product such as mRNA or protein.

Methods of detecting and/or measuring the concentration of protein and/or nucleic acid are well known to those skilled in the art, see for example Sambrook and Russell, 2001, Cold Spring Harbor Laboratory Press.

Preferred methods for detection and/or measurement of protein include Western blot, North-Western blot, immunosorbent assays (ELISA), antibody microarray, tissue microarray (TMA), immunoprecipitation, in situ hybridisation and other immunohistochemistry techniques, radioimmunoassay (RIA), immunoradiometric assays (IRMA) and immunoenzymatic assays (IEMA), including sandwich assays using monoclonal and/or polyclonal antibodies. Exemplary sandwich assays are described by David et al., in U.S. Pat. Nos. 4,376,110 and 4,486,530, hereby incorporated by reference. Antibody staining of cells on slides may be used in methods well known in cytology laboratory diagnostic tests, as well known to those skilled in the art.

Typically, ELISA involves the use of enzymes which give a coloured reaction product, usually in solid phase assays. Enzymes such as horseradish peroxidase and phosphatase have been widely employed. A way of amplifying the phosphatase reaction is to use NADP as a substrate to generate NAD which now acts as a coenzyme for a second enzyme system. Pyrophosphatase from Escherichia coli provides a good conjugate because the enzyme is not present in tissues, is stable and gives a good reaction colour. Chemi-luminescent systems based on enzymes such as luciferase can also be used.

Conjugation with the vitamin biotin is frequently used since this can readily be detected by its reaction with enzyme-linked avidin or streptavidin to which it binds with great specificity and affinity.

Preferred methods for detection and/or measurement of nucleic acid (e.g. mRNA) include southern blot, northern blot, polymerase chain reaction (PCR), reverse transcriptase PCR (RT-PCR), quantitative real-time PCR (qRT-PCR), nanoarray, microarray, macroarray, autoradiography and in situ hybridisation.

In one embodiment of the first aspect of the invention step (b) comprises measuring the expression of a nucleic acid molecule encoding the one or more biomarker(s). The nucleic acid molecule may be a cDNA molecule or an mRNA molecule. Preferably the nucleic acid molecule is an mRNA molecule. Also preferably the nucleic acid molecule is a cDNA molecule.

Hence, measuring the expression of the one or more biomarker(s) in step (b) may be performed using a method selected from the group consisting of Southern hybridisation, Northern hybridisation, polymerase chain reaction (PCR), reverse transcriptase PCR (RT-PCR), quantitative real-time PCR (qRT-PCR), nanoarray, microarray, macroarray, autoradiography and in situ hybridisation. Preferably measuring the expression of the one or more biomarker(s) in step (b) is determined using a DNA microarray. Hence, the method may comprise or consist of measuring the expression of the one or more biomarker(s) in step (b) using one or more binding moiety, each capable of binding selectively to a nucleic acid molecule encoding one of the biomarkers identified in Table 1.

Preferably the one or more binding moieties each comprise or consist of a nucleic acid molecule such as DNA, RNA, PNA, LNA, GNA, TNA or PMO (preferably DNA). Preferably the one or more binding moieties are 5 to 100 nucleotides in length. More preferably, the one or more nucleic acid molecules are 15 to 35 nucleotides in length. The binding moiety may comprise a detectable moiety.

Suitable binding agents (also referred to as binding molecules) may be selected or screened from a library based on their ability to bind a given nucleic acid, protein or amino acid motif, as discussed below.

In another embodiment of the first aspect of the invention step (b) comprises measuring the expression of the protein or polypeptide of the one or more biomarker(s) or a fragment or derivative thereof. Preferably measuring the expression of the one or more biomarker(s) in step (b) is performed using one or more binding moieties each capable of binding selectively to one of the biomarkers identified in Table 1.

The one or more binding moieties may comprise or consist of an antibody or an antigen-binding fragment thereof.

The term “antibody” includes any synthetic antibodies, recombinant antibodies or antibody hybrids, such as but not limited to, a single-chain antibody molecule produced by phage-display of immunoglobulin light and/or heavy chain variable and/or constant regions, or other immunointeractive molecules capable of binding to an antigen in an immunoassay format that is known to those skilled in the art.

We also include the use of antibody-like binding agents, such as affibodies and aptamers.

A general review of the techniques involved in the synthesis of antibody fragments which retain their specific binding sites is to be found in Winter & Milstein (1991) Nature 349, 293-299.

Additionally, or alternatively, one or more of the first binding molecules may be an aptamer (see Collett et al., 2005, Methods 37:4-15).

Molecular libraries such as antibody libraries (Clackson et al, 1991, Nature 352, 624-628; Marks et al, 1991, J Mol Biol 222(3): 581-97), peptide libraries (Smith, 1985, Science 228(4705): 1315-7), expressed cDNA libraries (Santi et al (2000) J Mol Biol 296(2): 497-508), libraries on other scaffolds than the antibody framework such as affibodies (Gunneriusson et al, 1999, Appl Environ Microbiol 65(9): 4134-40) or libraries based on aptamers (Kenan et al, 1999, Methods Mol Biol 118, 217-31) may be used as a source from which binding molecules that are specific for a given motif are selected for use in the methods of the invention.

The molecular libraries may be expressed in vivo in prokaryotic cells (Clackson et al, 1991, op. cit.; Marks et al, 1991, op. cit.) or eukaryotic cells (Kieke et al, 1999, Proc Natl Acad Sci USA, 96(10):5651-6) or may be expressed in vitro without involvement of cells (Hanes & Pluckthun, 1997, Proc Natl Acad Sci USA 94(10):4937-42; He & Taussig, 1997, Nucleic Acids Res 25(24):5132-4; Nemoto et al, 1997, FEBS Lett, 414(2):405-8).

In cases when protein based libraries are used, the genes encoding the libraries of potential binding molecules are often packaged in viruses and the potential binding molecule displayed at the surface of the virus (Clackson et al, 1991, supra; Marks et al, 1991, supra; Smith, 1985, supra).

Perhaps the most commonly used display system is filamentous bacteriophage displaying antibody fragments at their surfaces, the antibody fragments being expressed as a fusion to the minor coat protein of the bacteriophage (Clackson et al, 1991, supra; Marks et al, 1991, supra). However, other suitable systems for display include using other viruses (EP 39578), bacteria (Gunneriusson et al, 1999, supra; Daugherty et al, 1998, Protein Eng 11(9):825-32; Daugherty et al, 1999, Protein Eng 12(7):613-21), and yeast (Shusta et al, 1999, J Mol Biol 292(5):949-56).

In addition, display systems have been developed utilising linkage of the polypeptide product to its encoding mRNA in so-called ribosome display systems (Hanes & Pluckthun, 1997, supra; He & Taussig, 1997, supra; Nemoto et al, 1997, supra), or alternatively linkage of the polypeptide product to the encoding DNA (see U.S. Pat. No. 5,856,090 and WO 98/37186).

The variable heavy (V_{H}) and variable light (V_{L}) domains of the antibody are involved in antigen recognition, a fact first recognised by early protease digestion experiments. Further confirmation was found by “humanisation” of rodent antibodies. Variable domains of rodent origin may be fused to constant domains of human origin such that the resultant antibody retains the antigenic specificity of the rodent parented antibody (Morrison et al (1984) Proc. Natl. Acad. Sci. USA 81, 6851-6855).

That antigenic specificity is conferred by variable domains and is independent of the constant domains is known from experiments involving the bacterial expression of antibody fragments, all containing one or more variable domains. These molecules include Fab-like molecules (Better et al (1988) Science 240, 1041); Fv molecules (Skerra et al (1988) Science 240, 1038); single-chain Fv (ScFv) molecules where the V_{H} and V_{L} partner domains are linked via a flexible oligopeptide (Bird et al (1988) Science 242, 423; Huston et al (1988) Proc. Natl. Acad. Sci. USA 85, 5879) and single domain antibodies (dAbs) comprising isolated V domains (Ward et al (1989) Nature 341, 544). A general review of the techniques involved in the synthesis of antibody fragments which retain their specific binding sites is to be found in Winter & Milstein (1991) Nature 349, 293-299.

The antibody or antigen-binding fragment may be selected from the group consisting of intact antibodies, Fv fragments (e.g. single chain Fv and disulphide-bonded Fv), Fab-like fragments (e.g. Fab fragments, Fab′ fragments and F(ab)₂ fragments), single variable domains (e.g. V_{H} and V_{L} domains) and domain antibodies (dAbs, including single and dual formats [i.e. dAb-linker-dAb]). Preferably, the antibody or antigen-binding fragment is a single chain Fv (scFv).

The one or more binding moieties may alternatively comprise or consist of an antibody-like binding agent, for example an affibody or aptamer.

By “scFv molecules” we mean molecules wherein the V_{H} and V_{L} partner domains are linked via a flexible oligopeptide.

The advantages of using antibody fragments, rather than whole antibodies, are several-fold. The smaller size of the fragments may lead to improved pharmacological properties, such as better penetration of solid tissue. Effector functions of whole antibodies, such as complement binding, are removed. Fab, Fv, ScFv and dAb antibody fragments can all be expressed in and secreted from E. coli, thus allowing the facile production of large amounts of the said fragments.

Whole antibodies, and F(ab′)₂ fragments are “bivalent”. By “bivalent” we mean that the said antibodies and F(ab′)₂ fragments have two antigen combining sites. In contrast, Fab, Fv, ScFv and dAb fragments are monovalent, having only one antigen combining sites.

The antibodies may be monoclonal or polyclonal. Suitable monoclonal antibodies may be prepared by known techniques, for example those disclosed in “Monoclonal Antibodies: A manual of techniques”, H Zola (CRC Press, 1988) and in “Monoclonal Hybridoma Antibodies: Techniques and applications”, J G R Hurrell (CRC Press, 1982), both of which are incorporated herein by reference.

When potential binding molecules are selected from libraries, one or more selector peptides having defined motifs are usually employed. Amino acid residues that provide structure, decreasing flexibility in the peptide or charged, polar or hydrophobic side chains allowing interaction with the binding molecule may be used in the design of motifs for selector peptides. For example:
(i) Proline may stabilise a peptide structure as its side chain is bound both to the alpha carbon as well as the nitrogen;
(ii) Phenylalanine, tyrosine and tryptophan have aromatic side chains and are highly hydrophobic, whereas leucine and isoleucine have aliphatic side chains and are also hydrophobic;
(iii) Lysine, arginine and histidine have basic side chains and will be positively charged at neutral pH, whereas aspartate and glutamate have acidic side chains and will be negatively charged at neutral pH;
(iv) Asparagine and glutamine are neutral at neutral pH but contain a amide group which may participate in hydrogen bonds;
(v) Serine, threonine and tyrosine side chains contain hydroxyl groups, which may participate in hydrogen bonds.

Typically, selection of binding molecules may involve the use of array technologies and systems to analyse binding to spots corresponding to types of binding molecules.

Hence, preferably the antibody or fragment thereof is a monoclonal antibody or fragment thereof. Preferably the antibody or antigen-binding fragment is selected from the group consisting of intact antibodies, Fv fragments (e.g. single chain Fv and disulphide-bonded Fv), Fab-like fragments (e.g. Fab fragments, Fab′ fragments and F(ab)₂ fragments), single variable domains (e.g. V_{H} and V_{L} domains) and domain antibodies (dAbs, including single and dual formats [i.e. dAb-linker-dAb]). Hence, the antibody or antigen-binding fragment may be a single chain Fv (scFv). Alternatively, the one or more binding moieties comprise or consist of an antibody-like binding agent, for example an affibody or aptamer. The one or more binding moieties comprise a detectable moiety.

By a “detectable moiety” we include a moiety which permits its presence and/or relative amount and/or location (for example, the location on an array) to be determined, either directly or indirectly.

Suitable detectable moieties are well known in the art.

For example, the detectable moiety may be a fluorescent and/or luminescent and/or chemiluminescent moiety which, when exposed to specific conditions, may be detected. Such a fluorescent moiety may need to be exposed to radiation (i.e. light) at a specific wavelength and intensity to cause excitation of the fluorescent moiety, thereby enabling it to emit detectable fluorescence at a specific wavelength that may be detected.

Alternatively, the detectable moiety may be an enzyme which is capable of converting a (preferably undetectable) substrate into a detectable product that can be visualised and/or detected. Examples of suitable enzymes are discussed in more detail below in relation to, for example, ELISA assays.

Hence, the detectable moiety may be selected from the group consisting of: a fluorescent moiety; a luminescent moiety; a chemiluminescent moiety; a radioactive moiety (for example, a radioactive atom); or an enzymatic moiety. Preferably, the detectable moiety comprises or consists of a radioactive atom. The radioactive atom may be selected from the group consisting of technetium-99m, iodine-123, iodine-125, iodine-131, indium-111, fluorine-19, carbon-13, nitrogen-15, oxygen-17, phosphorus-32, sulphur-35, deuterium, tritium, rhenium-186, rhenium-188 and yttrium-90.

Clearly, the agent to be detected (such as, for example, the one or more biomarkers in the test sample and/or control sample described herein and/or an antibody molecule for use in detecting a selected protein) must have sufficient of the appropriate atomic isotopes in order for the detectable moiety to be readily detectable.

In an alternative preferred embodiment, the detectable moiety of the binding moiety is a fluorescent moiety.

The radio- or other labels may be incorporated into the biomarkers present in the samples of the methods of the invention and/or the binding moieties of the invention in known ways. For example, if the binding agent is a polypeptide it may be biosynthesised or may be synthesised by chemical amino acid synthesis using suitable amino acid precursors involving, for example, fluorine-19 in place of hydrogen. Labels such as ^{99m}Tc, ¹²³I, ¹⁸⁶Rh, ¹⁸⁸Rh and ¹¹¹In can, for example, be attached via cysteine residues in the binding moiety. Yttrium-90 can be attached via a lysine residue. The IODOGEN method (Fraker et al (1978) Biochem. Biophys. Res. Comm. 80, 49-57) can be used to incorporate ¹²³I. Reference (“Monoclonal Antibodies in Immunoscintigraphy”, J-F Chatal, CRC Press, 1989) describes other methods in detail. Methods for conjugating other detectable moieties (such as enzymatic, fluorescent, luminescent, chemiluminescent or radioactive moieties) to proteins are well known in the art.

It will be appreciated by persons skilled in the art that biomarkers in the sample(s) to be tested may be labelled with a moiety which indirectly assists with determining the presence, amount and/or location of said proteins. Thus, the moiety may constitute one component of a multicomponent detectable moiety. For example, the biomarkers in the sample(s) to be tested may be labelled with biotin, which allows their subsequent detection using streptavidin fused or otherwise joined to a detectable label.

Detectable moieties may be selected from the group consisting of a fluorescent moiety, a luminescent moiety, a chemiluminescent moiety, a radioactive moiety and an enzymatic moiety.

Hence, the detectable moiety may comprise or consist of a radioactive atom. The radioactive atom may be selected from the group consisting of technetium-99m, iodine-123, iodine-125, iodine-131, indium-111, fluorine-19, carbon-13, nitrogen-15, oxygen-17, phosphorus-32, sulphur-35, deuterium, tritium, rhenium-186, rhenium-188 and yttrium-90.

Alternatively, the detectable moiety of the binding moiety may be a fluorescent moiety.

In the method according to the first aspect of the invention the samples provided in step (a) and/or step (c) are treated prior to step (b) and/or step (d), respectively, such that any biomarkers present in the samples may be labelled with biotin. Step (b) and/or step (d) may be performed using a detecting agent comprising Streptavidin and a detectable moiety (such as a fluorescent moiety).

Thus, the proteins of interest in the sample to be tested may first be isolated and/or immobilised using first binding agent(s), after which the presence and/or relative amount of said biomarkers may be determined using second binding agent(s).

In one embodiment, the second binding agent is an antibody or antigen-binding fragment thereof; typically a recombinant antibody or fragment thereof. Conveniently, the antibody or fragment thereof is selected from the group consisting of: scFv; Fab; a binding domain of an immunoglobulin molecule. Suitable antibodies and fragments, and methods for making the same, are described in detail above.

Alternatively, the second binding agent may be an antibody-like binding agent, such as an affibody or aptamer.

Alternatively, where the detectable moiety on the protein in the sample to be tested comprises or consists of a member of a specific binding pair (e.g. biotin), the second binding agent may comprise or consist of the complimentary member of the specific binding pair (e.g. streptavidin).

Where a detection assay is used, it is preferred that the detectable moiety is selected from the group consisting of: a fluorescent moiety; a luminescent moiety; a chemiluminescent moiety; a radioactive moiety; an enzymatic moiety. Examples of suitable detectable moieties for use in the methods of the invention are described above.

Preferred assays for detecting serum or plasma proteins include enzyme linked immunosorbent assays (ELISA), radioimmunoassay (RIA), immunoradiometric assays (IRMA) and immunoenzymatic assays (IEMA), including sandwich assays using monoclonal and/or polyclonal antibodies. Exemplary sandwich assays are described by David et al in U.S. Pat. Nos. 4,376,110 and 4,486,530, hereby incorporated by reference. Antibody staining of cells on slides may be used in methods well known in cytology laboratory diagnostic tests, as well known to those skilled in the art.

Thus, in one embodiment the assay is an ELISA (Enzyme Linked Immunosorbent Assay) which typically involves the use of enzymes which give a coloured reaction product, usually in solid phase assays. Enzymes such as horseradish peroxidase and phosphatase have been widely employed. A way of amplifying the phosphatase reaction is to use NADP as a substrate to generate NAD which now acts as a coenzyme for a second enzyme system. Pyrophosphatase from Escherichia coli provides a good conjugate because the enzyme is not present in tissues, is stable and gives a good reaction colour. Chemiluminescent systems based on enzymes such as luciferase can also be used.

Conjugation with the vitamin biotin is frequently used since this can readily be detected by its reaction with enzyme-linked avidin or streptavidin to which it binds with great specificity and affinity.

In an alternative embodiment, the assay used for protein detection is conveniently a fluorometric assay. Thus, the detectable moiety of the second binding agent may be a fluorescent moiety, such as an Alexa fluorophore (for example Alexa-647).

Preferably the predicative accuracy of the method, as determined by an ROC AUC value, is at least 0.50, for example at least 0.55, 0.60, 0.65, 0.70, 0.75, 0.80, 0.85, 0.90, 0.95, 0.96, 0.97, 0.98 or at least 0.99. More preferable the predicative accuracy of the method, as determined by an ROC AUC value, is at least 0.80 (most preferably 1).

In the method of the first aspect of the invention step (b) may be performed using an array such as a bead-based array or a surface-based array. Preferably the array is selected from the group consisting of: macroarray; microarray; nanoarray.

The method for determining a breast cancer-associated disease state may be performed using a support vector machine (SVM), such as those available from http://cran.r-project.org/web/packages/e1071/index.html (e.g. e1071 1.5-24). However, any other suitable means may also be used. SVMs may also be used to determine the ROC AUCs of biomarker signatures comprising or consisting of one or more Table 1 biomarkers as defined herein.

Support vector machines (SVMs) are a set of related supervised learning methods used for classification and regression. Given a set of training examples, each marked as belonging to one of two categories, an SVM training algorithm builds a model that predicts whether a new example falls into one category or the other. Intuitively, an SVM model is a representation of the examples as points in space, mapped so that the examples of the separate categories are divided by a clear gap that is as wide as possible. New examples are then mapped into that same space and predicted to belong to a category based on which side of the gap they fall on.

More formally, a support vector machine constructs a hyperplane or set of hyperplanes in a high or infinite dimensional space, which can be used for classification, regression or other tasks. Intuitively, a good separation is achieved by the hyperplane that has the largest distance to the nearest training datapoints of any class (so-called functional margin), since in general the larger the margin the lower the generalization error of the classifier. For more information on SVMs, see for example, Burges, 1998, Data Mining and Knowledge Discovery, 2:121-167.

In one embodiment of the invention, the SVM is ‘trained’ prior to performing the methods of the invention using biomarker profiles of known agents (namely, breast cancer cells of known histological grade or breast cancer cells from breast cancer patients with known distant metastasis-free survival). By running such training samples, the SVM is able to learn what biomarker profiles are associated with particular characteristics. Once the training process is complete, the SVM is then able whether or not the biomarker sample tested is from a particular breast cancer sample type (i.e., a particular breast cancer-associated disease state).

However, this training procedure can be by-passed by pre-programming the SVM with the necessary training parameters. For example, cells belonging to a particular breast cancer-associated disease state can be identified according to the known SVM parameters using the SVM algorithm detailed in Table 4, based on the measurement of the biomarkers listed in Table 1 using the values and/or regulation patterns detailed therein.

It will be appreciated by skilled persons that suitable SVM parameters can be determined for any combination of the biomarkers listed Table 1 by training an SVM machine with the appropriate selection of data (i.e. biomarker measurements from cells of known histological grade and/or cells from individuals with known metastasis-free survival times).

Alternatively, the Table 1 data may be used to determine a particular breast cancer-associated disease state according to any other suitable statistical method known in the art, such as Principal Component Analysis (PCA) and other multivariate statistical analyses (e.g., backward stepwise logistic regression model). For a review of multivariate statistical analysis see, for example, Schervish, Mark J. (November 1987). “A Review of Multivariate Analysis”. Statistical Science 2 (4): 396-413 which is incorporated herein by reference.

Preferably, the method of the invention has an accuracy of at least 65%, for example 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% accuracy.

Preferably, the method of the invention has a sensitivity of at least 65%, for example 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% sensitivity.

Preferably, the method of the invention has a specificity of at least 65%, for example 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% specificity.

By “accuracy” we mean the proportion of correct outcomes of a method, by “sensitivity” we mean the proportion of all positive chemicals that are correctly classified as positives, and by “specificity” we mean the proportion of all negative chemicals that are correctly classified as negatives.

The method of the first aspect of the invention may further comprise the steps of:
e) providing treatment to the individual being tested based upon the breast-cancer associated disease state determined in the preceding steps.

Hence, the method comprises treating the patient according to the histological grade of their breast cancer and/or according to their predicted metastasis-free survival time. For example, a more aggressive treatment may be provided for higher grade breast cancers and/or wherein metastasis-free survival time is predicted to be relatively low (e.g., less than 10 years) versus relatively high (e.g., more than 10 years). Suitable therapeutic approaches can be determined by the skilled person according to the prevailing guidance at the time, for example, see NICE Clinical Guideline 80 “Early and locally advanced breast cancer: Diagnosis and treatment”, (available here: http://www.nice.org.uk/nicemedia/pdf/CG80NICEGuideline.pdf) which is incorporated herein by reference.

Accordingly, the present invention comprises an antineoplastic agent for use in treating breast cancer wherein the dosage regime is determined based on the results of the method of the first aspect of the invention.

The present invention comprises the use of an antineoplastic agent in treating breast cancer wherein the dosage regime is determined based on the results of the method of the first aspect of the invention.

The present invention comprises the use of an antineoplastic agent in the manufacture of a medicament for treating breast cancer wherein the dosage regime is determined based on the results of the method of the first aspect of the invention.

The present invention comprises a method of treating breast cancer comprising providing a sufficient amount of an antineoplastic agent wherein the amount of antineoplastic agent sufficient to treat the breast cancer is determined based on the results of the method of the first aspect of the invention.

In one embodiment, the antineoplastic agent is an alkylating agent (ATC code L01a), an antimetabolite (ATC code L01b), a plant alkaloid or other natural product (ATC code L01c), a cytotoxic antibiotic or a related substance (ATC code L01d), or an other antineoplastic agents (ATC code L01x).

Hence, in one embodiment the antineoplastic agent is an alkylating agent selected from the group consisting of a nitrogen mustard analogue (for example cyclophosphamide, chlorambucil, melphalan, chlormethine, ifosfamide, trofosfamide, prednimustine or bendamustine) an alkyl sulfonate (for example busulfan, treosulfan, or mannosulfan) an ethylene imine (for example thiotepa, triaziquone or carboquone) a nitrosourea (for example carmustine, lomustine, semustine, streptozocin, fotemustine, nimustine or ranimustine) an epoxides (for example etoglucid) or another alkylating agent (ATC code L01ax, for example mitobronitol, pipobroman, temozolomide or dacarbazine).

In a another embodiment the antineoplastic agent is an antimetabolite selected from the group consisting of a folic acid analogue (for example methotrexate, raltitrexed, pemetrexed or pralatrexate), a purine analogue (for example mercaptopurine, tioguanine, cladribine, fludarabine, clofarabine or nelarabine) or a pyrimidine analogue (for example cytarabine, fluorouracil, tegafur, carmofur, gemcitabine, capecitabine, azacitidine or decitabine).

In a still further embodiment the antineoplastic agent is a plant alkaloid or other natural product selected from the group consisting of a vinca alkaloid or a vinca alkaloid analogue (for example vinblastine, vincristine, vindesine, vinorelbine or vinflunine), a podophyllotoxin derivative (for example etoposide or teniposide) a colchicine derivative (for example demecolcine), a taxane (for example paclitaxel, docetaxel or paclitaxel poliglumex) or another plant alkaloids or natural product (ATC code L01cx, for example trabectedin).

In one embodiment the antineoplastic agent is a cytotoxic antibiotic or related substance selected from the group consisting of an actinomycine (for example dactinomycin), an anthracycline or related substance (for example doxorubicin, daunorubicin, epirubicin, aclarubicin, zorubicin, idarubicin, mitoxantrone, pirarubicin, valrubicin, amrubicin or pixantrone) or another (ATC code L01dc, for example bleomycin, plicamycin, mitomycin or ixabepilone).

In a further embodiment the antineoplastic agent is an other antineoplastic agent selected from the group consisting of a platinum compound (for example cisplatin, carboplatin, oxaliplatin, satraplatin or polyplatillen) a methylhydrazine (for example procarbazine) a monoclonal antibody (for example edrecolomab, rituximab, trastuzumab, alemtuzumab, gemtuzumab, cetuximab, bevacizumab, panitumumab, catumaxomab or ofatumumab) a sensitizer used in photodynamic/radiation therapy (for example porfimer sodium, methyl aminolevulinate, aminolevulinic acid, temoporfin or efaproxiral) or a protein kinase inhibitor (for example imatinib, gefitinib, erlotinib, sunitinib, sorafenib, dasatinib, lapatinib, nilotinib, temsirolimus, everolimus, pazopanib, vandetanib, afatinib, masitinib or toceranib).

In a still further embodiment the antineoplastic agent is an other neoplastic agent selected from the group consisting of amsacrine, asparaginase, altretamine, hydroxycarbamide, lonidamine, pentostatin, miltefosine, masoprocol, estramustine, tretinoin, mitoguazone, topotecan, tiazofurine, irinotecan, alitretinoin, mitotane, pegaspargase, bexarotene, arsenic trioxide, denileukin diftitox, bortezomib, celecoxib, anagrelide, oblimersen, sitimagene ceradenovec, vorinostat, romidepsin, omacetaxine mepesuccinate or eribulin.

Accordingly, a second aspect of the invention provides an array for use in a method according to the first aspect of the invention, the array comprising one or more first binding agents as defined above in relation to the first aspect of the invention.

The array binding agents may comprise or consist of binding agents which are collectively capable of binding to one or more biomarkers selected from the group defined in Table 1A, for example at least 2, biomarkers selected from the group defined in Table 1A. Preferably the array binding agents may comprise or consist of binding agents which are collectively capable of binding to one or more biomarkers selected from the group defined in Table 1B, for example at least 2, 3, 4, 5, 6, 7, 8, 9, 10, 11, 12, 13, 14, 15, 16, 17, 18, 19, 20, 21, 22, 23, 24, 25, 26, 27, 28, 29 or at least 30 biomarkers selected from the group defined in Table 1B. Preferably the array binding agents may comprise or consist of binding agents which are collectively capable of binding to one or more biomarkers selected from the group defined in Table 10, for example at least 2, 3, 4, 5, 6, 7, 8, 9, 10, 11, 12, 13, 14, 15, 16, 17, 18, 19, 20, 21, 22, 23, 24, 25, 26, 27 or at least 28 biomarkers selected from the group defined in Table 10. Preferably the array binding agents may comprise or consist of binding agents which are collectively capable of binding to one or more biomarkers selected from the group defined in Table 1D, for example at least 2, 3, 4, 5, 6, 7, 8, 9 or at least 10 biomarkers selected from the group defined in Table 1D. Preferably the array binding agents may comprise or consist of binding agents which are collectively capable of binding to one or more biomarkers selected from the group defined in Table 1E, for example at least 2, 3, 4, 5, 6, 7, 8 or at least 9 biomarkers selected from the group defined in Table 1E.

Hence, the array binding agents may comprise or consist of binding agents which are collectively capable of binding to all of the biomarkers defined in Table 1A. The array binding agents may comprise or consist of binding agents which are collectively capable of binding to all of the biomarkers defined in Table 1B. The array binding agents may comprise or consist of binding agents which are collectively capable of binding to all of the biomarkers defined in Table 1C. The array binding agents may comprise or consist of binding agents which are collectively capable of binding to all of the biomarkers defined in Table 1D. The array binding agents may comprise or consist of binding agents which are collectively capable of binding to all of the biomarkers defined in Table 1E. Preferably the array binding agents comprise or consist of binding agents which are collectively capable of binding to all of the biomarkers defined in Table 1.

The first binding agents of the array may be immobilised.

Arrays per se are well known in the art. Typically they are formed of a linear or two-dimensional structure having spaced apart (i.e. discrete) regions (“spots”), each having a finite area, formed on the surface of a solid support. An array can also be a bead structure where each bead can be identified by a molecular code or colour code or identified in a continuous flow. Analysis can also be performed sequentially where the sample is passed over a series of spots each adsorbing the class of molecules from the solution. The solid support is typically glass or a polymer, the most commonly used polymers being cellulose, polyacrylamide, nylon, polystyrene, polyvinyl chloride or polypropylene. The solid supports may be in the form of tubes, beads, discs, silicon chips, microplates, polyvinylidene difluoride (PVDF) membrane, nitrocellulose membrane, nylon membrane, other porous membrane, non-porous membrane (e.g. plastic, polymer, perspex, silicon, amongst others), a plurality of polymeric pins, or a plurality of microtitre wells, or any other surface suitable for immobilising proteins, polynucleotides and other suitable molecules and/or conducting an immunoassay. The binding processes are well known in the art and generally consist of cross-linking covalently binding or physically adsorbing a protein molecule, polynucleotide or the like to the solid support. Alternatively, affinity coupling of the probes via affinity-tags or similar constructs may be employed. By using well-known techniques, such as contact or non-contact printing, masking or photolithography, the location of each spot can be defined. For reviews see Jenkins, R. E., Pennington, S. R. (2001, Proteomics, 2, 13-29) and Lal et al (2002, Drug Discov Today 15; 7(18 Suppl):S143-9).

Typically the array is a microarray. By “microarray” we include the meaning of an array of regions having a density of discrete regions of at least about 100/cm², and preferably at least about 1000/cm². The regions in a microarray have typical dimensions, e.g. diameter, in the range of between about 10-250 μm, and are separated from other regions in the array by about the same distance. The array may alternatively be a macroarray or a nanoarray.

Once suitable binding molecules (discussed above) have been identified and isolated, the skilled person can manufacture an array using methods well known in the art of molecular biology; see Examples below.

A third aspect of the invention provides the use of one or more biomarkers selected from the group defined in Table 1A, Table 1B, Table 10, Table 1D and/or Table 1D for determining a breast cancer-associated disease state.

In one embodiment all of the biomarkers defined in Table 1A, Table 1B, Table 10, Table 1D and Table 1D are used collectively for determining a breast cancer-associated disease state.

A fourth aspect of the invention provide an analytical kit for use in a method according to the first aspect of the invention comprising:
A) an array according to the second aspect of the invention or as defined in the first aspect of the invention; and
B) instructions for performing the method as defined in the first aspect of the invention (optional).

The analytical kit may comprise one or more control samples as defined in the first aspect of the invention.

Preferred, non-limiting examples which embody certain aspects of the invention will now be described, with reference to the following figures:

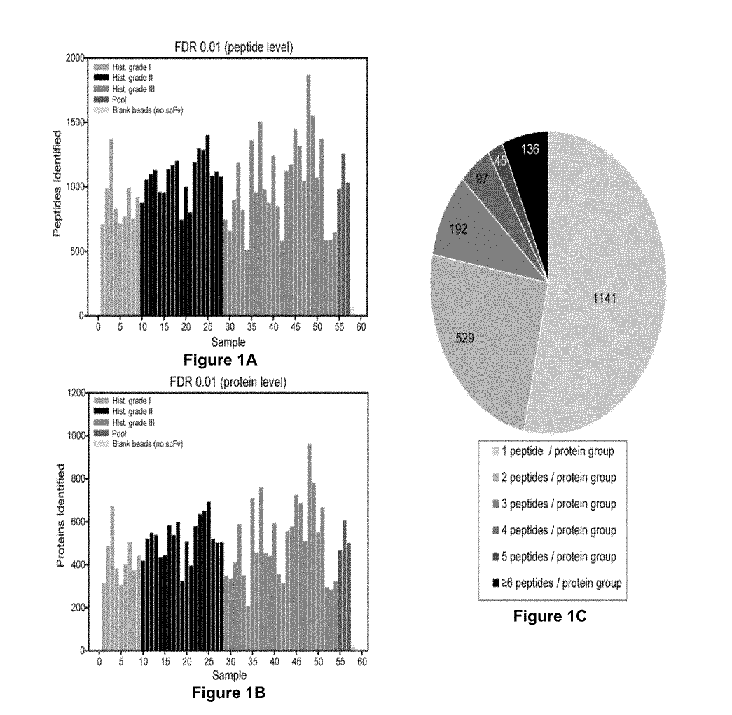

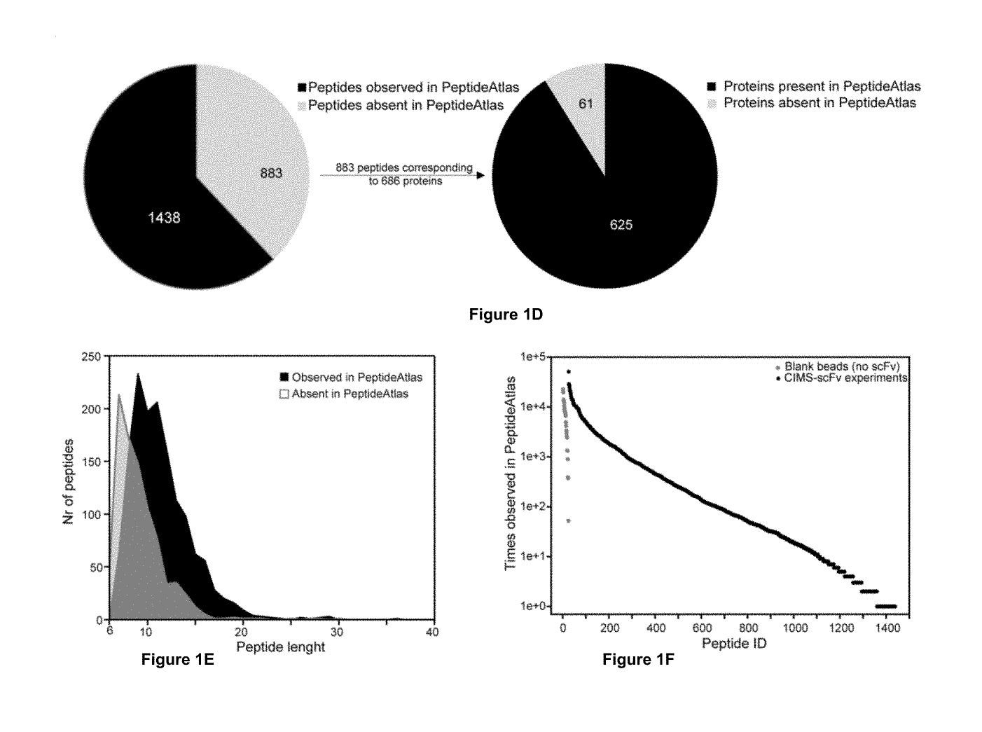

FIGS. 1A-1F. Peptide and protein statistics. (FIG. 1A) Total number of unique peptide sequences identified per sample (FDR 0.01, using Mascot+X!Tandem). (FIG. 1B) Total number of assembled protein groups identified per sample (FDR 0.01, set at protein level, using Mascot+X!Tandem). (FIG. 10) Number of unique peptides per protein group (FDR 0.01, set at protein level, using Mascot+X!Tandem) resulting in a total protein coverage of 2140 protein groups in the entire study. (Data based on all samples and runs, including replicates, pool runs and samples with missing clinical parameters). (FIG. 1D) Evaluation of quantified peptides (Progenesis LC-MS software, limited to Mascot scored peptides using FDR 0.01) against the PeptideAtlas (version 2011-08 Ens62, human). In addition, for peptides not present in the PeptideAtlas, a second comparison was performed in order to evaluate if the corresponding protein had been reported. In cases of multiple protein accessions, all were assessed. (FIG. 1E) Comparison of peptide length. (FIG. 1F) Observed peptide frequency in PeptideAtlas.

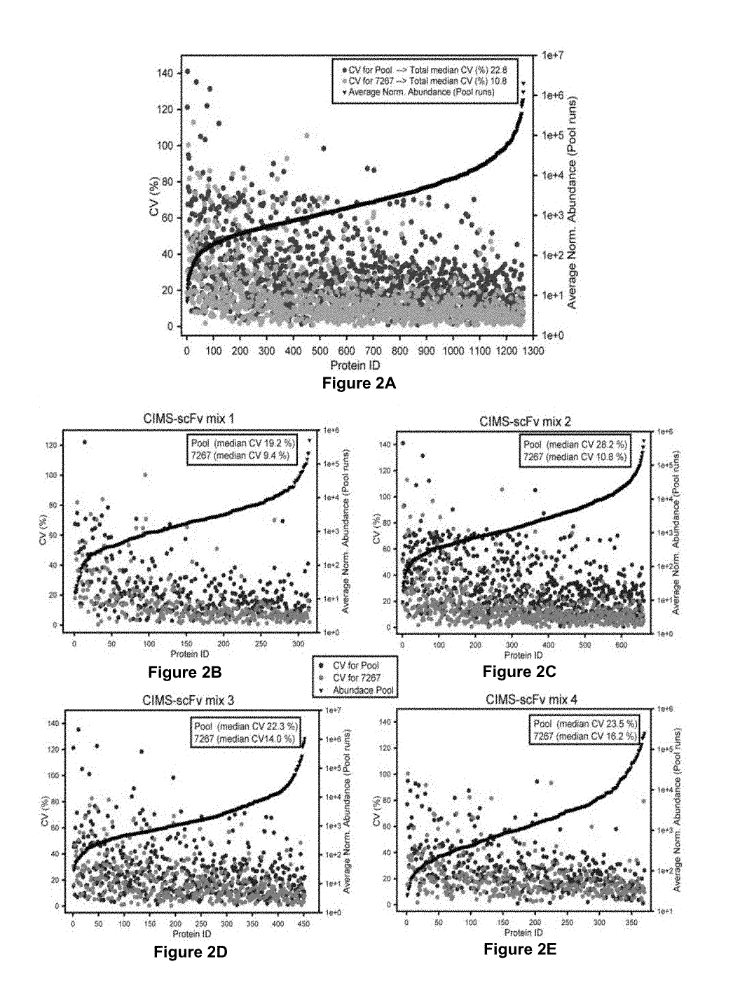

FIGS. 2A-2E. Reproducibility of the entire GPS setup (i.e. capture+LC-MS/MS) illustrated both for combined data and individual mixtures for a representative sample (sample 7267) and the reference (pool) sample. In order to include (plot) a data point, the protein had to be quantified (normalized abundance >0) in all triplicate runs. Such requirement was used for all data plotted in FIGS. 2A-2E. (FIG. 2A) Illustrated for all data combined (based on 1264 proteins). (FIG. 2B) Illustrated for CIMS-mix 1 (based on 315 proteins) (FIG. 2C) Illustrated for CIMS-mix 2 (based on 661 proteins) (FIG. 2D) Illustrated for CIMS-mix 3 (based on 452 proteins) (FIG. 2E) Illustrated for CIMS-mix 4 (based on 370 proteins).

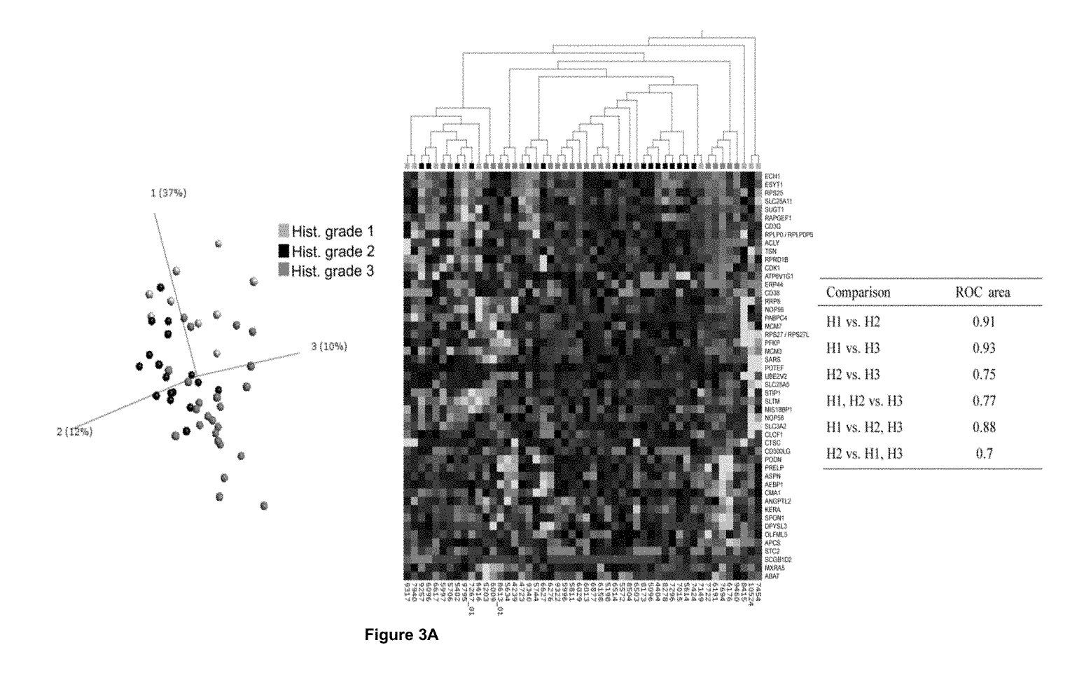

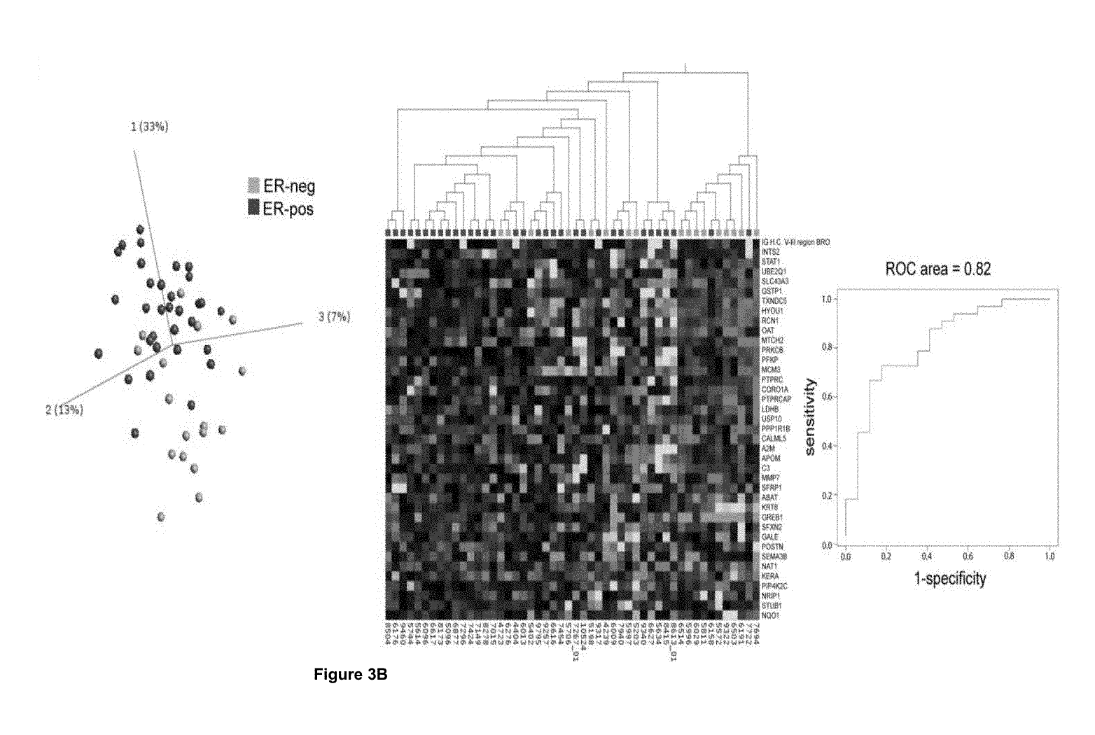

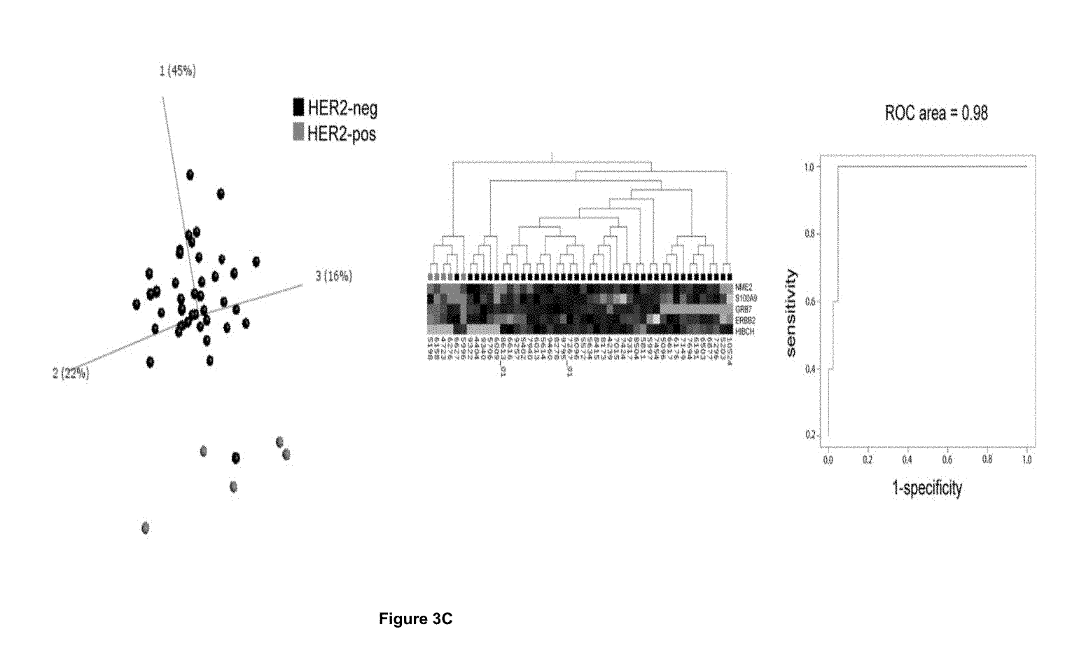

FIGS. 3A-3C. Significantly differentially expressed proteins based on histologic grade, estrogen receptor status, and HER2 status. Differentially expressed analytes are shown in heatmaps (red—up-regulated, green—down-regulated). (FIG. 3A) PCA-plot and associated heatmap of histologic grade 1, grade 2 and grade 3 samples (data filtered on variance 0.2, p-value <0.01, q-value <0.25). In addition results from a leave-one out cross validation approach with a SVM demonstrated with ROC-area values. (FIG. 3B) PCA-plot and associated heatmap of ER-positive and ER-negative samples (data filtered on variance 0.2, p-value <0.01, q-value <0.32). In addition result from a leave-one out cross validation approach illustrated with a ROC-curve. (FIG. 3C) PCA-plot and associated heatmap of HER2-positive and negative samples. (data filtered on variance 0.2, p-value <0.01, q-value <0.9). Result from a leave-one out cross validation approach illustrated with a ROC-curve.

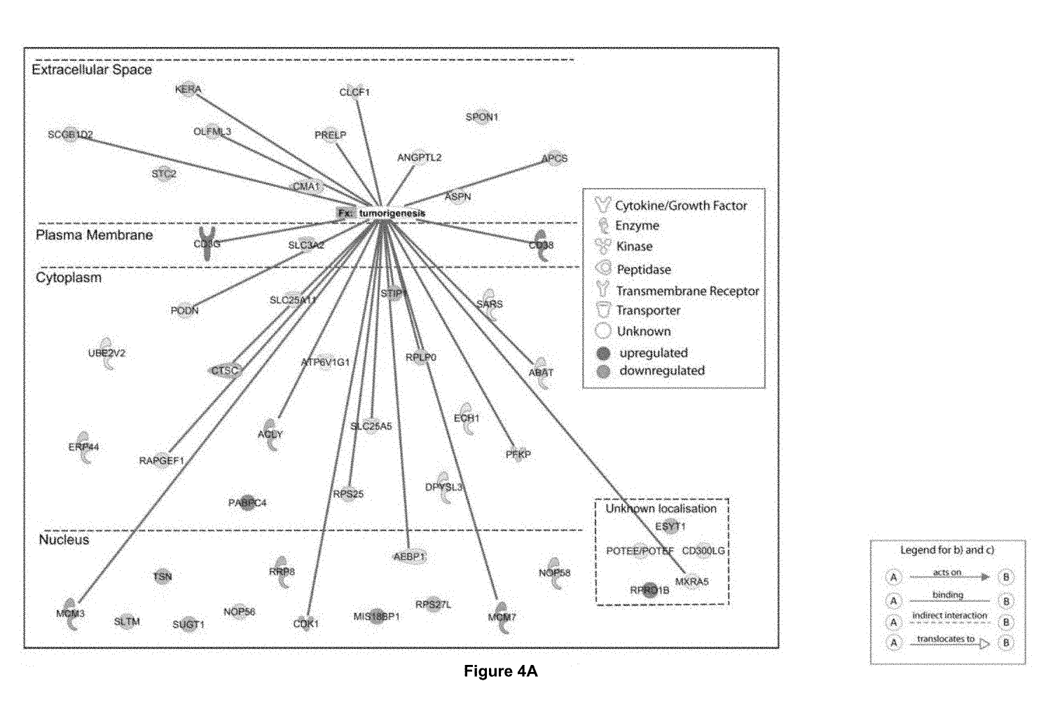

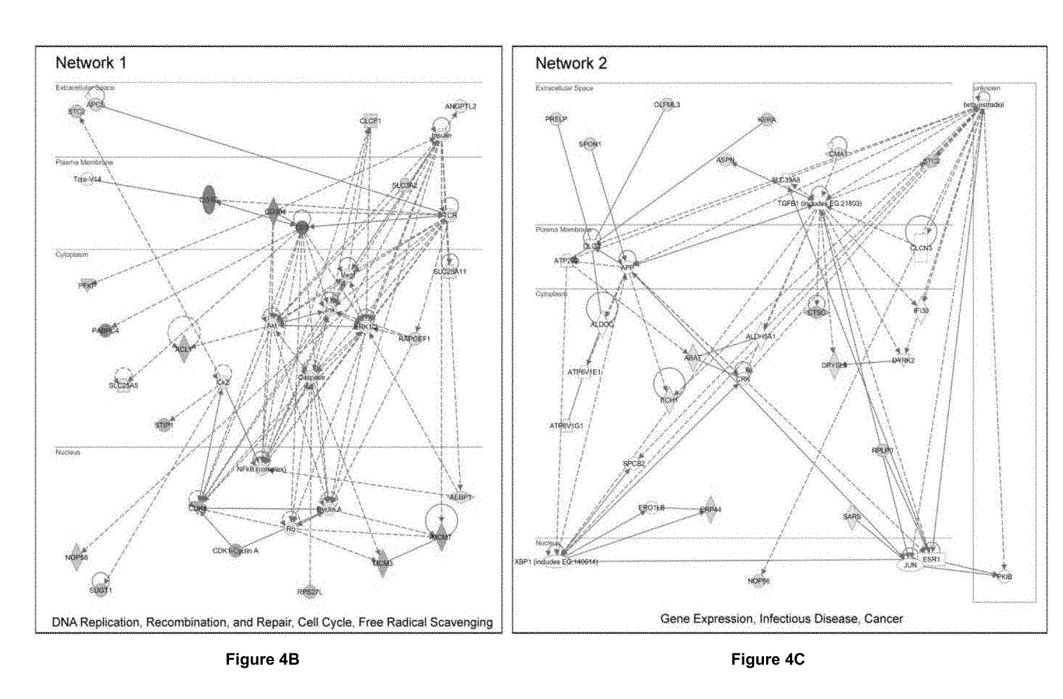

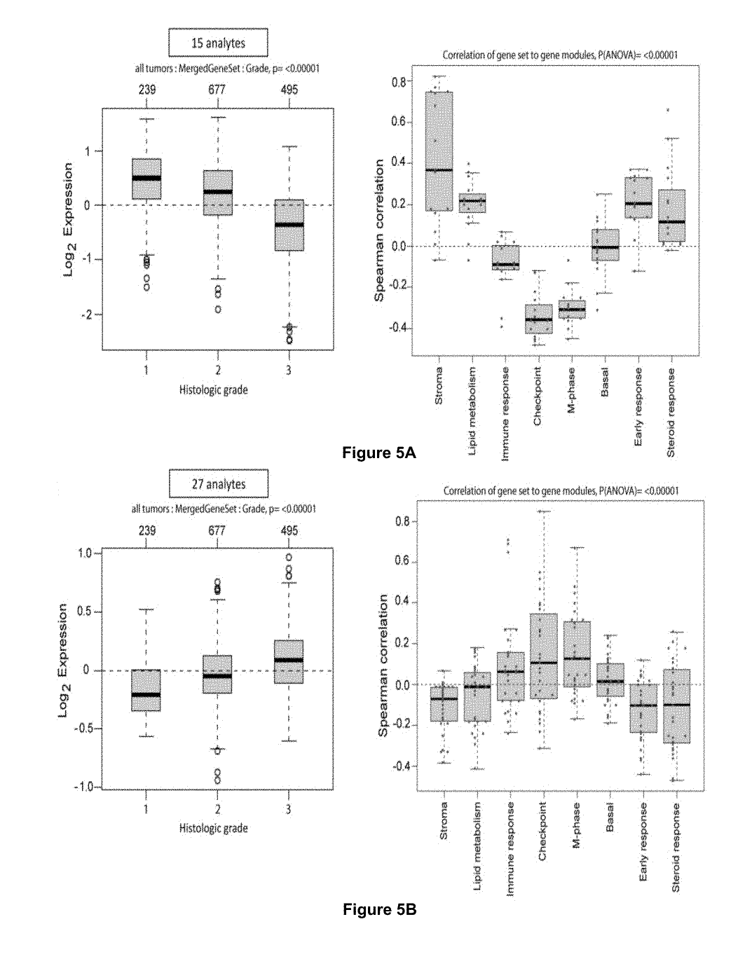

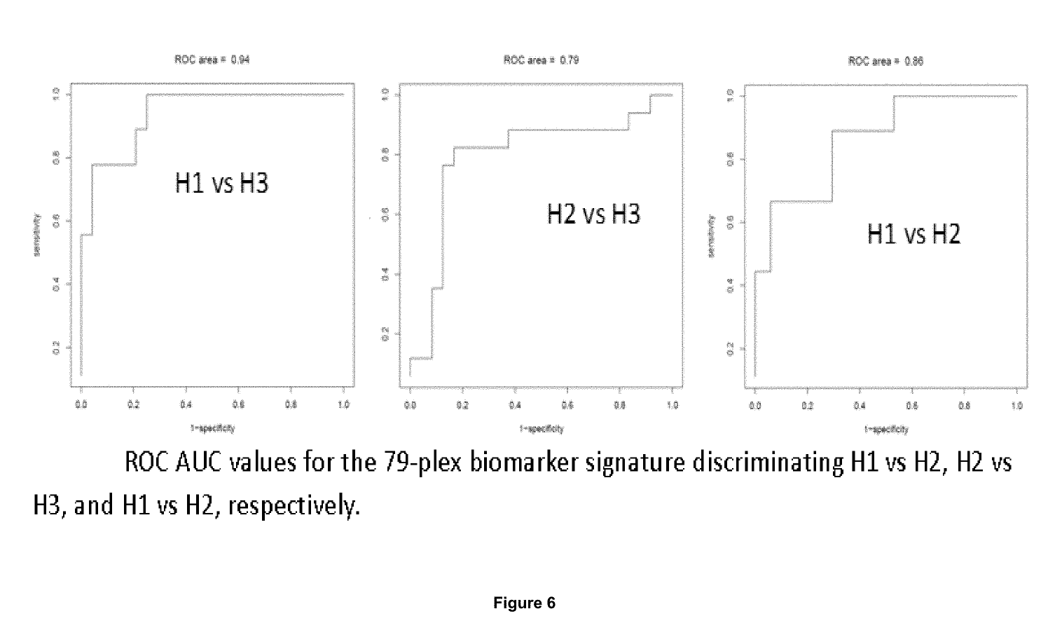

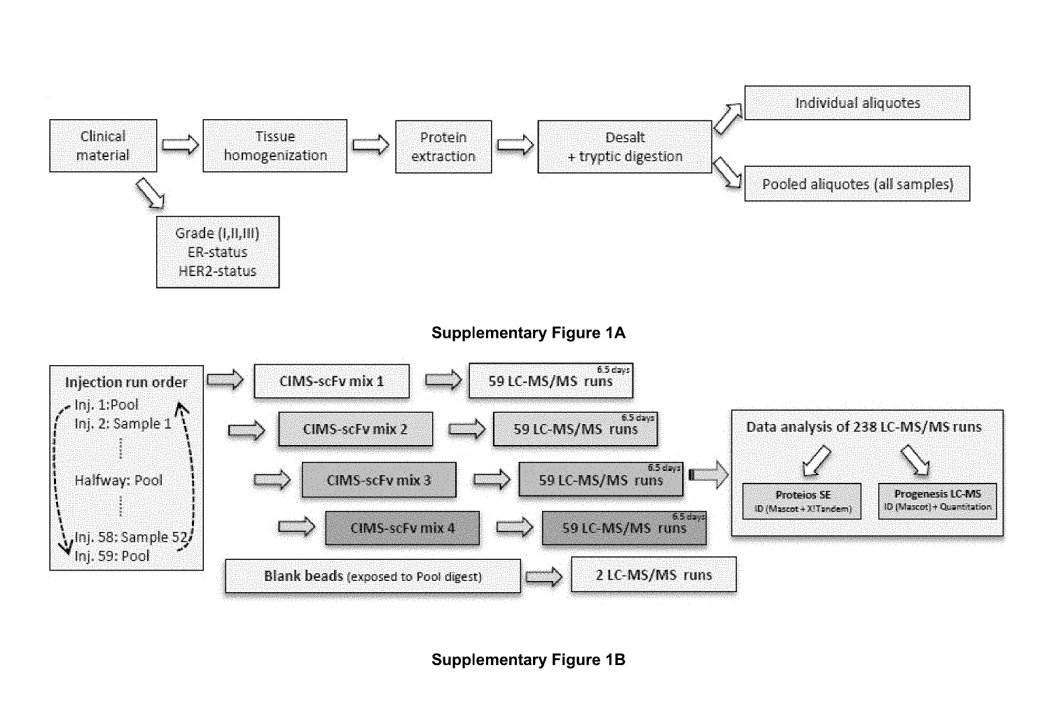

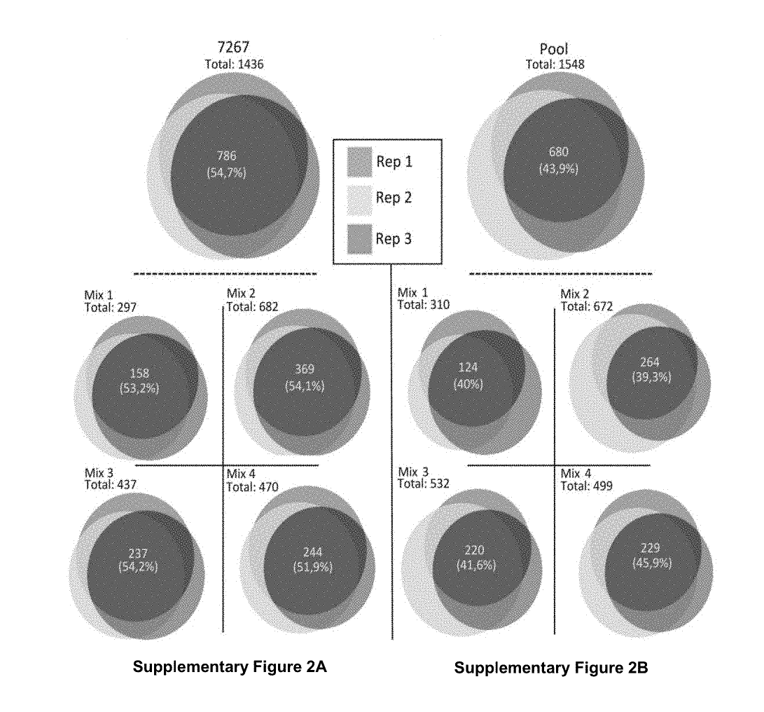

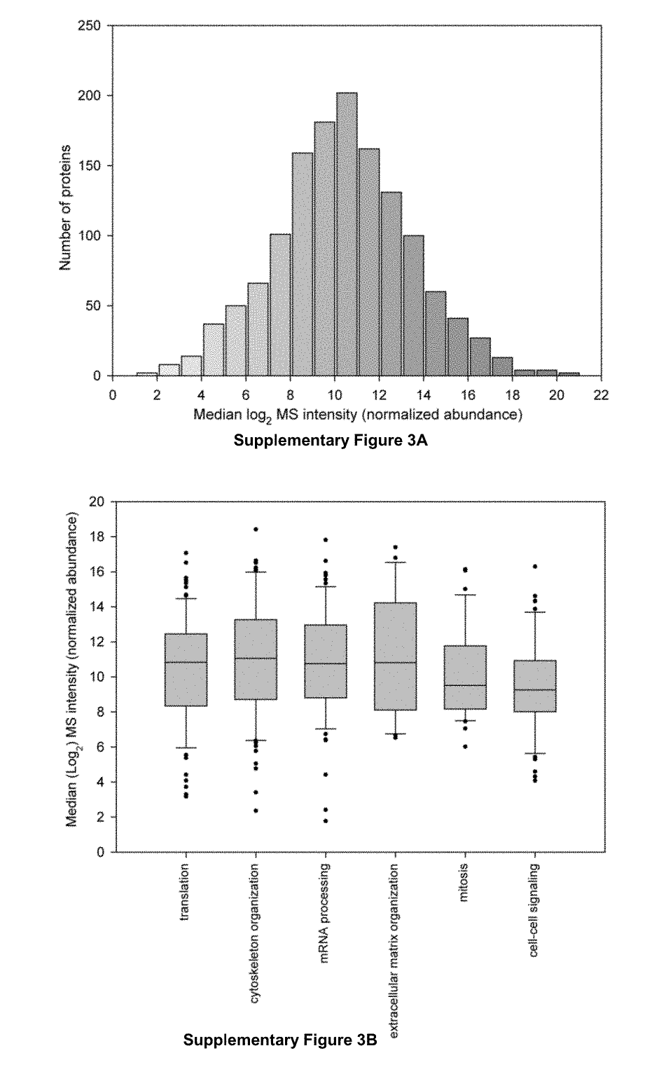

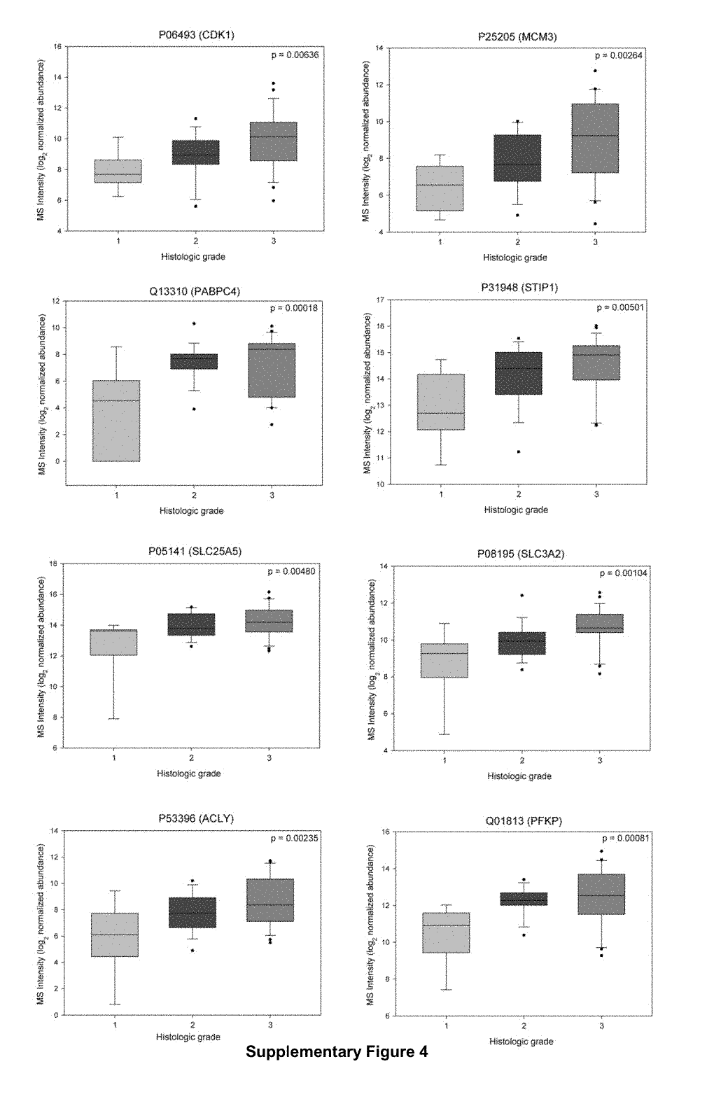

FIGS. 4A-4C. Biological relevance of differentially expressed analytes between the three histologic graded tumor types using IPA. (FIG. 4A) The 49 proteins identified as significantly differentially expressed proteins between the three tumor cohorts mapped to their cellular localization. Colored log₂-ratio (median grade 3/median grade 1) where red color illustrates up-regulation and green color illustrates down-regulation. Proteins with known association to tumorigenesis have been indicated. (FIG. 4B) The top reported network found to be associated with DNA replication, recombination, cell cycle, and free radical scavenging. (FIG. 4C) The second reported network found to be associated with gene expression, infectious disease, and cancer.

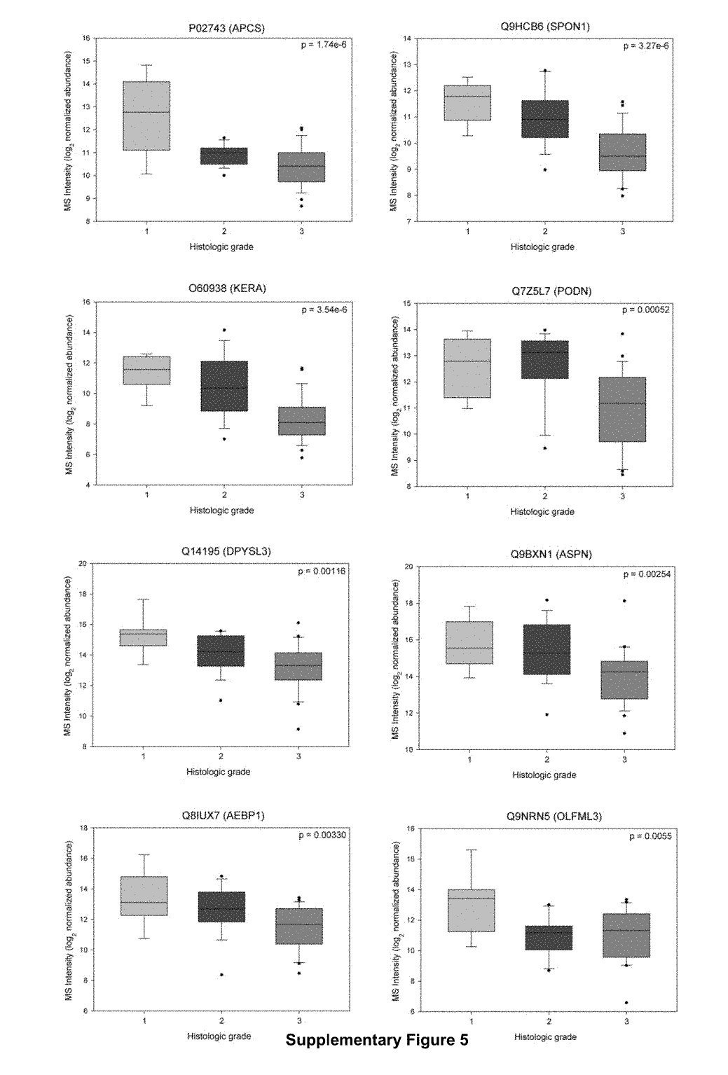

FIGS. 5A-5B. Validation of protein expression profiles using an orthogonal method. To this end, mRNA expression profiles based on data from 1411 histological graded tumor samples was used. 42 of 49 differentially expressed proteins among histologic grade 1, 2 and 3 were successfully mapped (using Gene Entrez ID) into the GOBO-database. (FIG. 5A) mRNA expression profiles for proteins found to display a decreased protein expression in histologic grade 3 tumors (median ratio compared to histologic grade 1) whereof 15 (of total 16) analytes could be mapped with the GOBO-tool. In addition, correlation of the 15 genes to different gene set module expression pattern is indicated. Grey dots indicate actual correlation values. (FIG. 5B) mRNA expression profiles for proteins found to display an increased expression in histologic grade 3 tumors (compared to histologic grade 1) whereof 27 (of total 33) could be mapped with the GOBO-tool. In addition, correlation of the 27 genes to different gene set module expression pattern is indicated. Grey dots indicate actual correlation values.

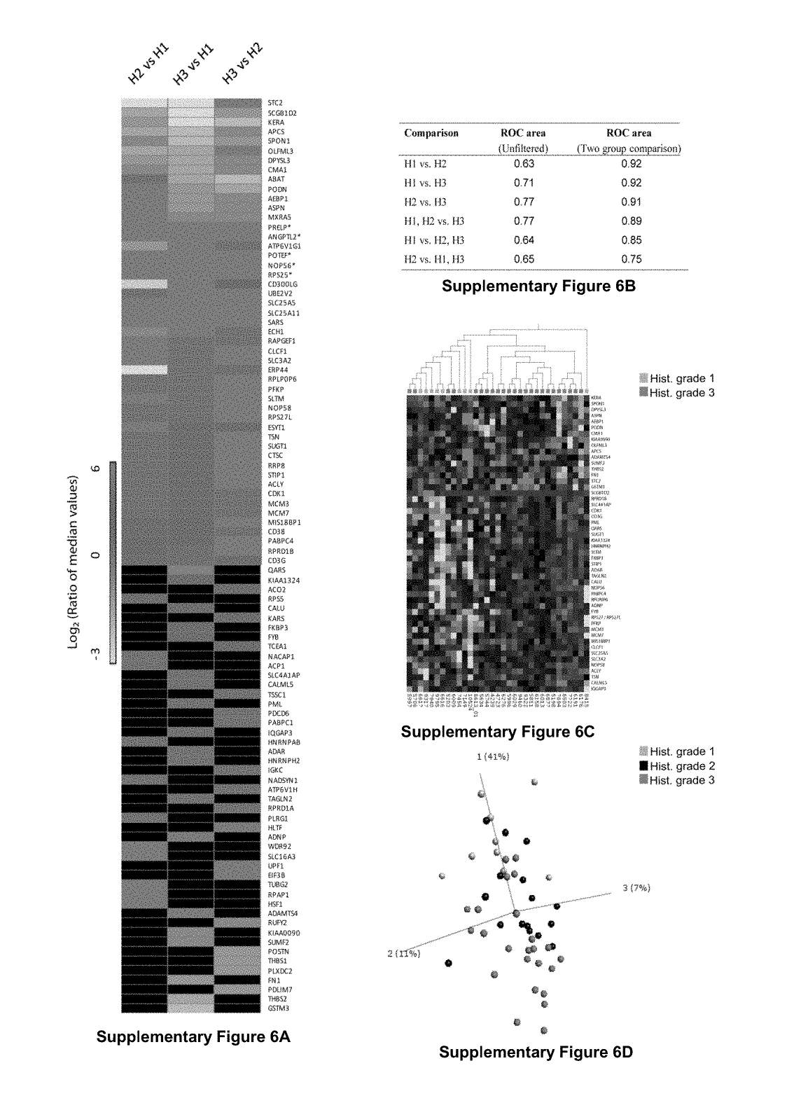

FIG. 6. kaplan meier analyses of exemplary biomarker signatures of varying lengths and combinations of Table 1 biomarkers.

FIGS. S1A-S1B. Schematic overview of the workflow used in the study. (FIG. S1A) Tumor sample preparations and (FIG. S1B) peptide capture using CIMS-antibodies, run schedule on the LC-MS/MS and data analysis. The analysis of all eluates derived from one CIMS-binder mix were finalised prior to move on to the next CIMS-binder mix derived eluates. Consequently, all CIMS-binder mix analysis started with an analysis of an eluate from the pooled sample, continuing with half of the individual samples in a random sequence according to histological grade. Halfway through the mix, another pool sample was injected, then the remaining samples and at the end finishing with the third pool sample. After completion, the analysis of the eluates from the next CIMS-binder mix was started. Between binder mix 2 and binder mix 3, two injections of blank beads were run. Blank beads contained no antibodies attached, so only background peptides that bind to the magnetic beads should elute. Data was analyzed in Proteios SE and Progenisis to obtain identification and quantification of peptides and proteins.

FIGS. S2A-S2B. Identification reproducibility of the entire GPS setup (i.e. capture +LC-MS/MS) illustrated as Venn diagrams. (FIG. S2A) Overlap of peptides (all unique sequences) between replicate capture runs for sample 7267. Statistics for total coverage of sample 7267 (top diagram) and individual mixes (the smaller four Venn diagrams) are shown. Data generated from Proteios SE (i.e. Mascot and X!Tandem scored peptides). (FIG. S2B) Overlap of peptides (all unique sequences) between replicate capture runs for the pool sample. Statistics for both total (top diagram) and individual mixes are shown. Data generated from Proteios SE (i.e. Mascot and X!Tandem scored peptides).

FIGS. S3A-S3B. Distribution of log 2 MS intensity for quantified proteins. (FIG. S3A) Median normalized abundance (based on 50 samples with clinical records) plotted for 1364 proteins (24 proteins with a median log 2 intensity value of 0 were excluded). Bars are colored according to MS intensity, ranging from light yellow (low MS intensity) to dark red (high MS intensity). (FIG. S3B) The distribution of log 2 MS intensity values based on GO biological processes for selected protein categories. Analytes were grouped by major biological processes using the Generic Gene Ontology (GO) Term Mapper tool (http://go.princeton.edu/cgi-bin/GOTermMapper).

FIG. S4. Individual intensity boxplots for 8 of the differentially expressed proteins between the three histological grades, demonstrating highest expression in histological grade 3 tumors.

FIG. S5. Individual intensity boxplots for 8 of the differentially expressed proteins between the three histological grades, demonstrating highest expression in histological grade 1 tumors.

FIGS. S6A-S6D. Extended comparisons between histological grades (FIG. S6A) log 2-fold change between histologic grade 2 (H2) and histologic grade 1 (H1), between histologic grade 3 (H3) and histologic grade 1 (H1), and between histologic grade 3 (H3) and histologic grade 2 (H2). The top 49 illustrated analytes are the protein signature identified as differentially expressed between the three grades. Therefore, all comparisons are calculated and shown. The lower 47 analytes are derived from SVM-calculations between two of the grades while the third grade is left out. This calculation was done for all three comparisons, and the list of significant analytes was consequently compiled. The matrix color figure was generated using Matrix2png (Pavlidis and Noble, 2003). (FIG. S6B) The ROC AUC values derived from the SVM calculations from a two group comparison. Listed are both ROC AUC values from unfiltered, (entire dataset) as well as filtered data (variance 0.2 and p-value <0.01). (FIG. S6C) Heatmap of histological grade 1 and grade 3 (data filtered on variance 0.2, p-value <0.01, q-value <0.25). Differentially expressed analytes are shown in heatmaps, where red color illustrates up-regulation and green color illustrates down-regulation. (FIG. S6D) PCA-plot of histological grade 1, grade 2 and grade 3 using the 50 differentially expressed proteins between grade 1 and grade 3.

FIGS. S7A-S7B. Individual intensity boxplots exemplified for a subset of proteins identified as differentially expressed in the ER-status comparison or the HER2/neu-status comparison. (FIG. S7A) Differentially expressed analytes between ER-positive and ER-negative tumors. (FIG. S7B) Differentially expressed analytes between HER2-negative and HER2-positive tumors.

FIGS. S8A-S8B. Evaluation of Ki67-positive (25% cut off) and Ki67-negative staged tumors. Differentially expressed analytes are shown in heatmaps, where red color illustrates up-regulation and green color illustrates down-regulation. (FIG. S8A) PCA-plot of Ki67-positive and Ki67-negative staged tumors. Heatmap of corresponding analytes and samples. (data filtered on variance 0.2, p-value <0.01, q-value <0.27). (FIG. S8B) Result from a leave-one out cross validation approach with a SVM illustrated with a ROC-curve.

FIGS. S9A-S9B. Transcription factor association network analysis using IPA for the differentially expressed analytes reflecting histological grade or ER-status. Lines connecting molecules indicate molecular relationships and the style of the arrows indicate specific molecular relationships and the directionality of the interaction. (FIG. S9A) The 49 proteins identified in the multi-group histological grad comparison were used as input. Log₂ ratio of the median value for histological grade 3 vs histological grade 1 used in order to color code measured analytes. Red color illustrates up regulation. Green color illustrates down-regulation. (FIG. S9B) The 39 proteins identified in the ER-status comparison were used as input. Log₂ ratio of the median value used in order to colour code measured analytes. Red colour illustrates up regulation and green colour illustrates down-regulation in the ER-negative samples.

FIGS. S10A-S10J. Individual mRNA expression profiles based on data from 1411 histological graded tumor samples exemplified for a subset of the analytes found to display significant differential protein expression between histologic grades. (FIGS. S10A-S10E) mRNA expression levels for five proteins found to display increased expression in histologic grade 3 tumors. (FIGS. S10E-S10J) mRNA expression levels for five proteins found to display decreased expression in histologic grade 3 tumors.

FIGS. S11A-S11F. mRNA expression profiles based on data from 1620 ER-status defined breast tumor samples. 32 of the 39 differentially expressed proteins were successfully mapped into the GOBO-database using gene entrez ID. (FIG. S11A) mRNA expression profiles for 10 proteins found to display an increased protein expression in ER-positive tumors. In addition correlation of the 10 genes to different gene set module expression pattern is illustrated. Grey dots indicate actual correlation values. (FIG. S11B) mRNA expression profiles for proteins found to display an decreased expression in ER-positive tumors. In addition correlation of the 22 genes to different gene set module expression pattern can be seen. Grey dots indicate actual correlation values. (FIGS. S11C-S11D) Individual mRNA expression profiles exemplified for two proteins found to display increased expression in ER-positive tumors. (FIGS. S11E-S11F) Individual mRNA expression profiles exemplified for two proteins found to display decreased expression in ER-positive tumors.

FIGS. S12A-S12C. Individual mRNA expression profiles mapped, using the GOBO-database tool, exemplified for three analytes found to display significant differential protein expression in the HER2/neu comparison. Data based on 1881 available tumor samples. (FIG. S12A) HER2/neu, (FIG. S12B) S100A9 (FIG. S12C) GRB7. In addition to above three analytes a fourth protein (accession P22392) was tested to be mapped. However, due to error message from the GOBO-database tool using either Gene Entrez ID 4831 or 654364 the data is missing.

FIG. S13. Kaplan-Meier analysis, using DMFS as 10-year endpoint. 42 of the 49 proteins differentiating the histological grades were successfully mapped to the gene expression database using Entrez Gene ID (after converting swissprot ID). The analytes were divided into two groups (based on up- or down-regulation, using a ratio between histological grade 3 and grade 1 samples for the observed protein expression level) resulting in 15 down-regulated analytes and 27 up-regulated analytes. These two groups were then used to assess potential risk of distant metastasis free survival (DMFS) using the gene expression dataset. Kaplan-Meier analysis, using DMFS as 10-year endpoint for histological graded tumors (n=1379) was performed by stratifying the gene expression data into three quantiles. In addition, four individual Kaplan-Meier analysis using DMFS as 10-year endpoint based on single genes (2 down-regulated and two up-regulated) were generated and displayed in a similar manner using the GOBO-tool.

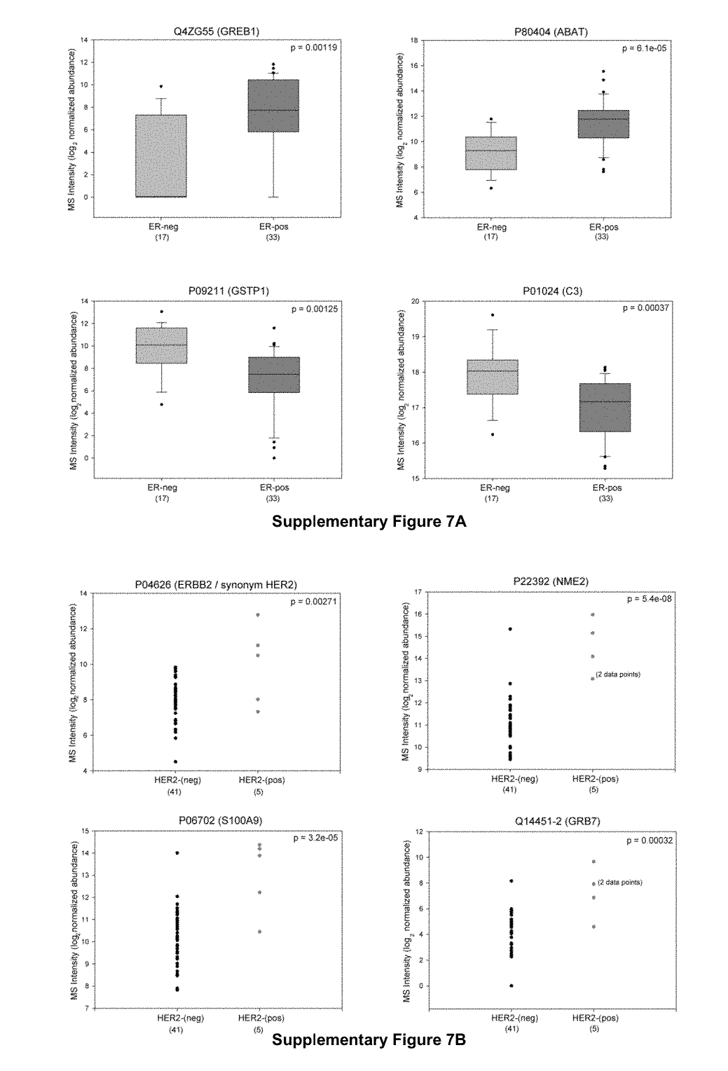

FIG. 7. Breast cancer tissue samples were selected from the same original cohort of 52 samples, here including 6 grade 1 samples, 9 grade 2 samples and 6 grade 9 samples. The samples were digested (trypsinated) in solution and analysed using Selective Monitoring Reaction (SRM) set-up (an established mass spectrometry based approach). 9 peptides corresponding to 8 proteins from the stated list of biomarkers were targeted and quantified. Samples were run in triplicate. Data was analysed using Anubis Followed by P-value filtering (p<0.01) and q-value filtering (q<0.11). The data shows that the breast cancer tissues samples could be differentiated according to grade using a truncated list of markers.

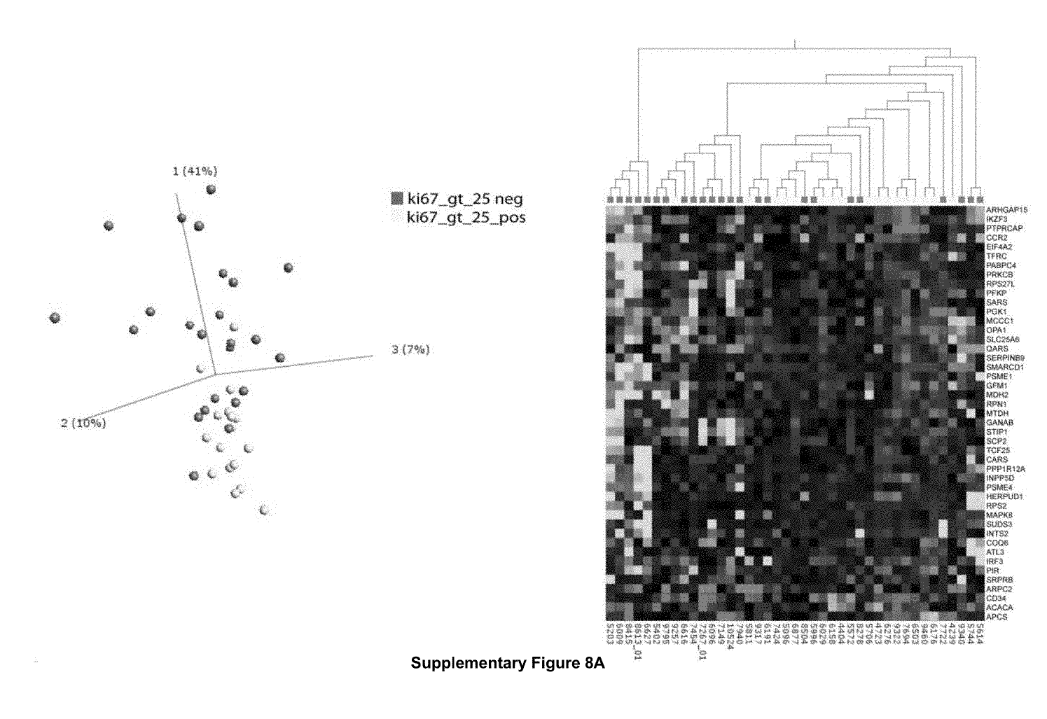

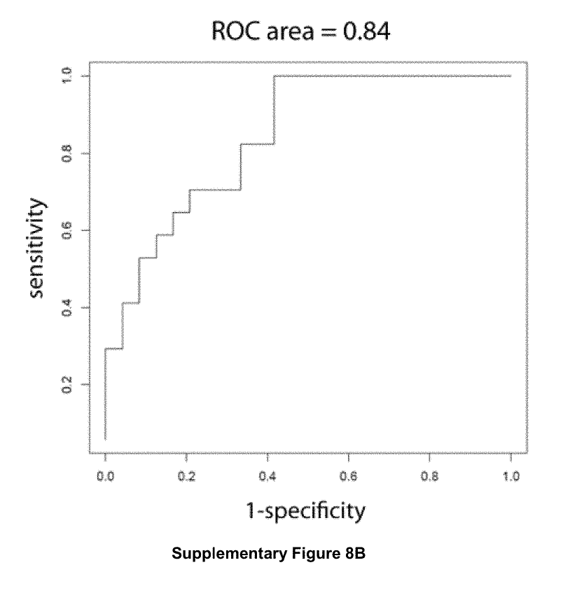

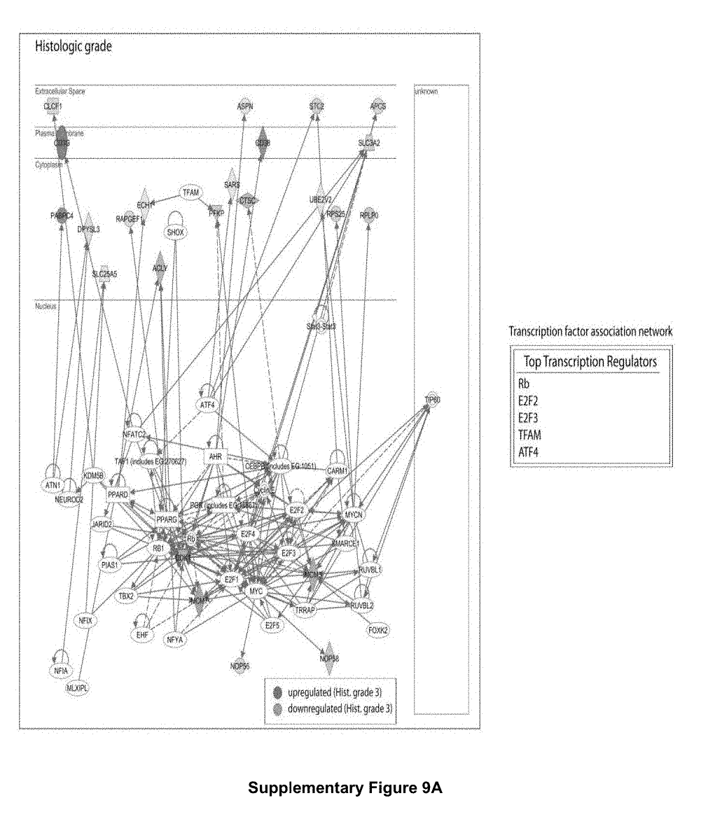

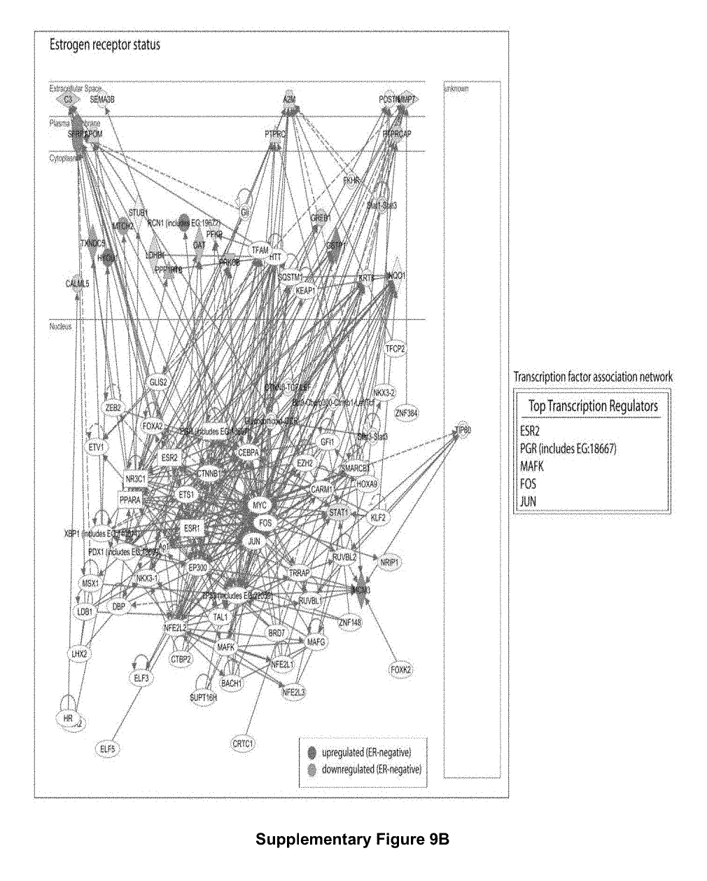

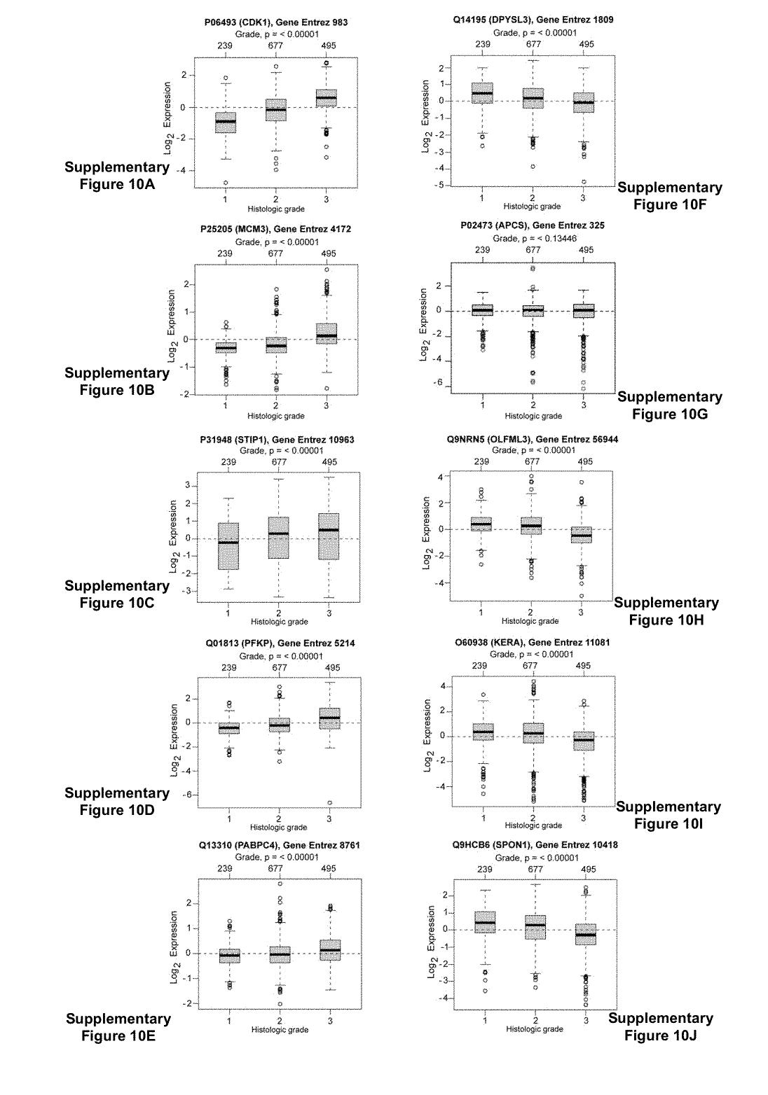

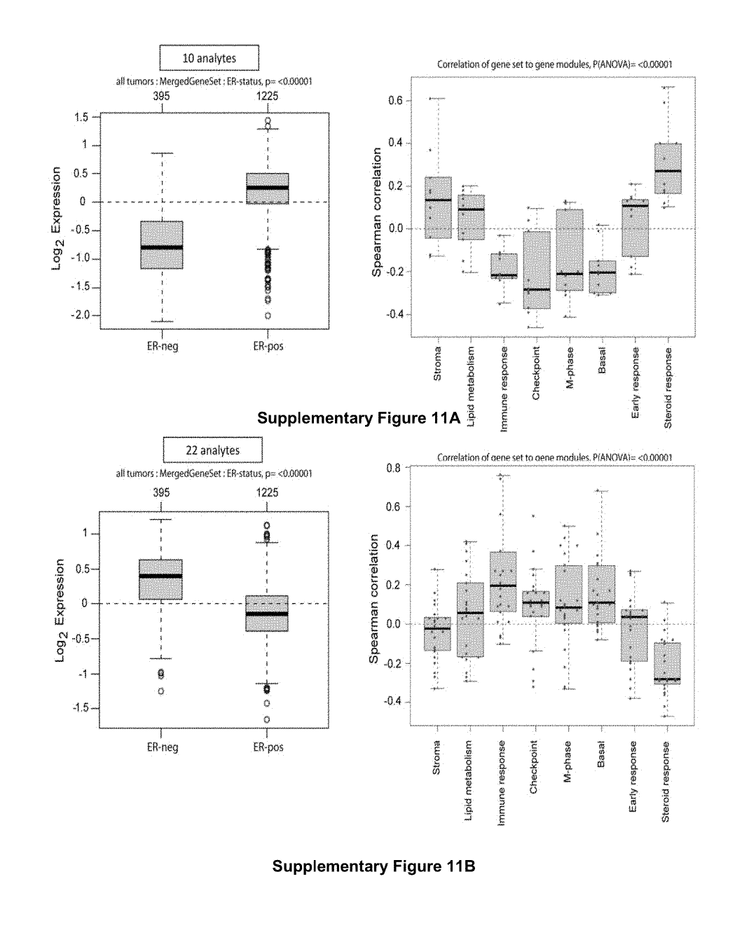

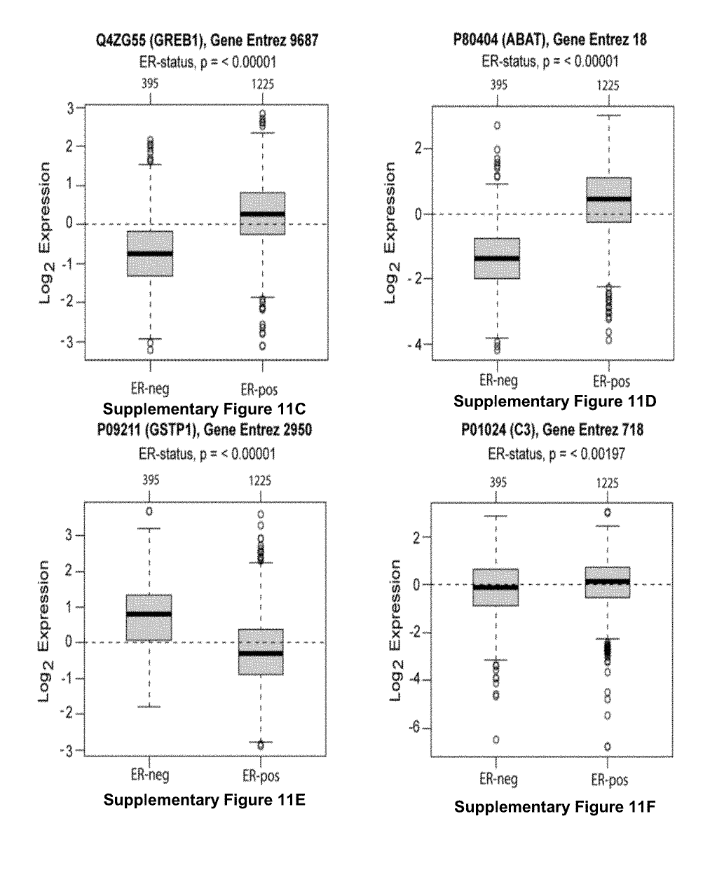

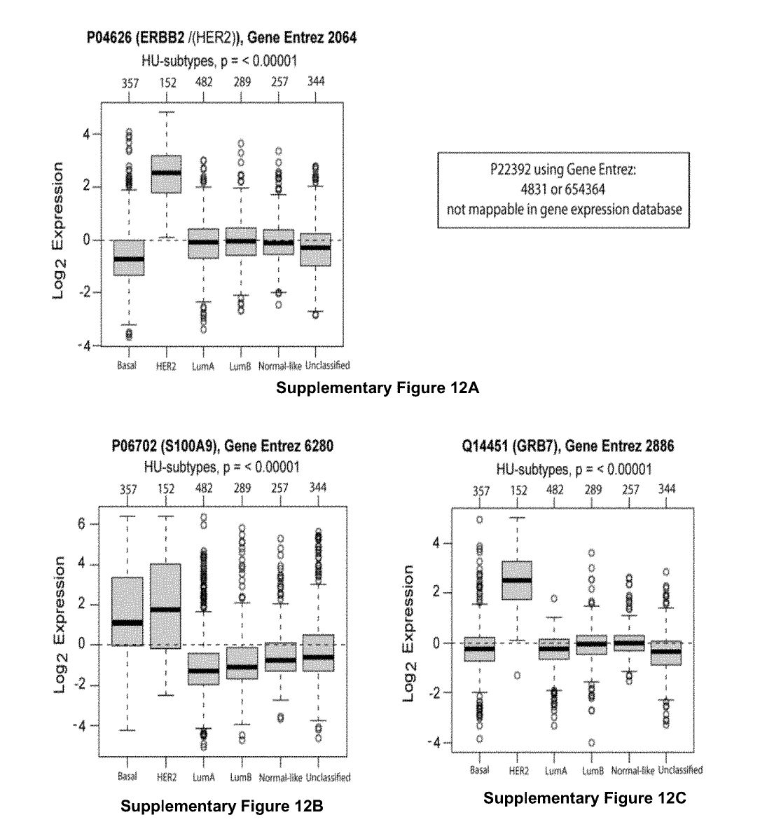

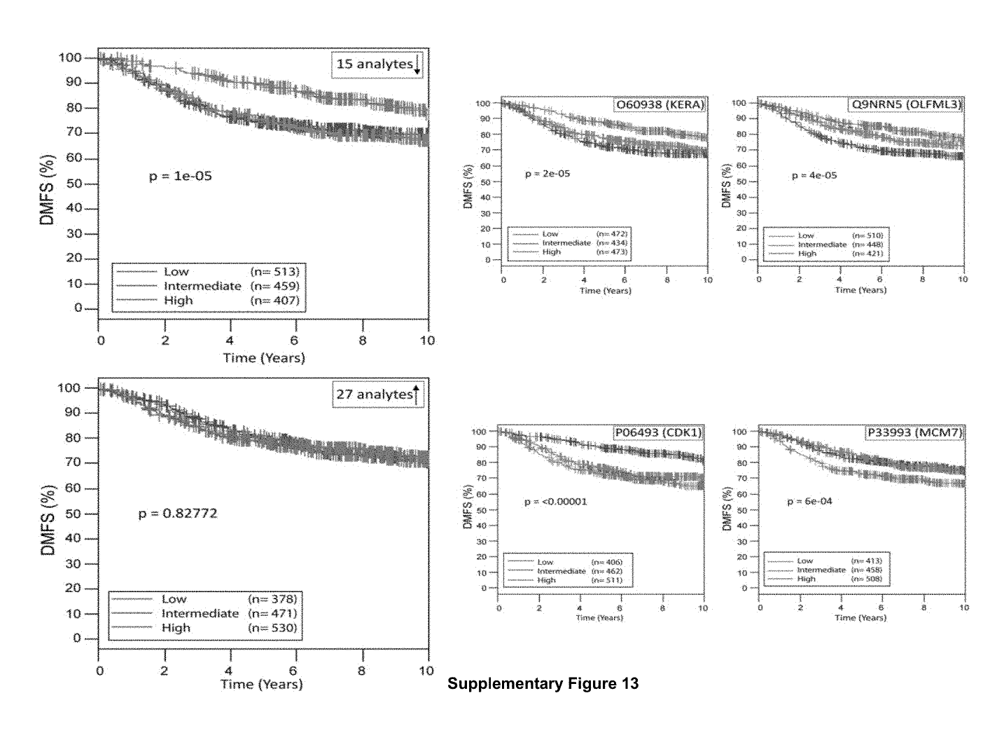

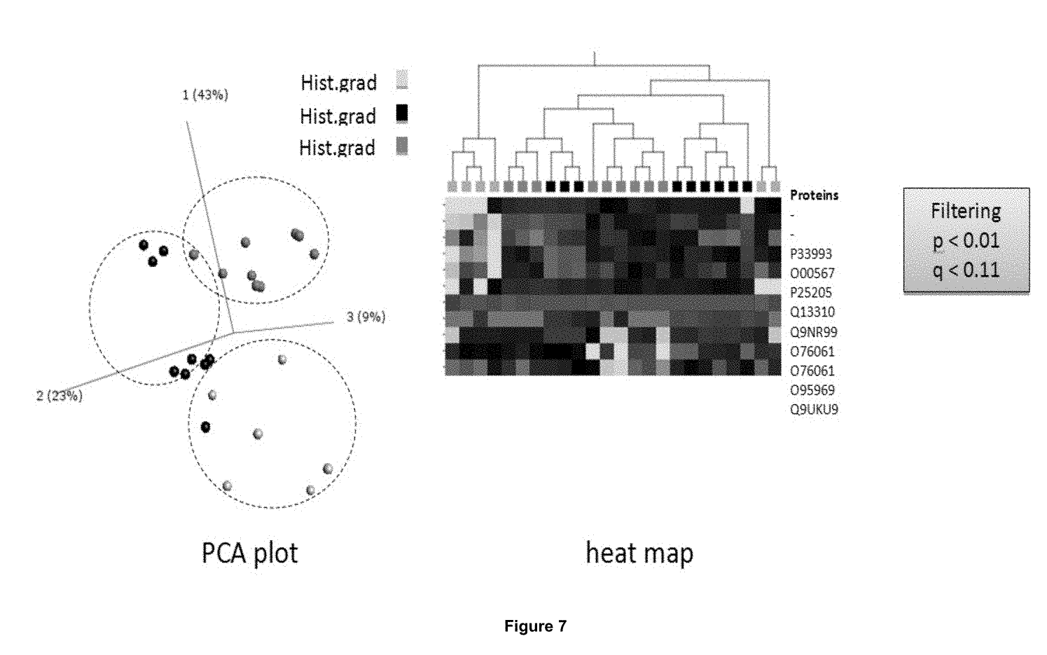

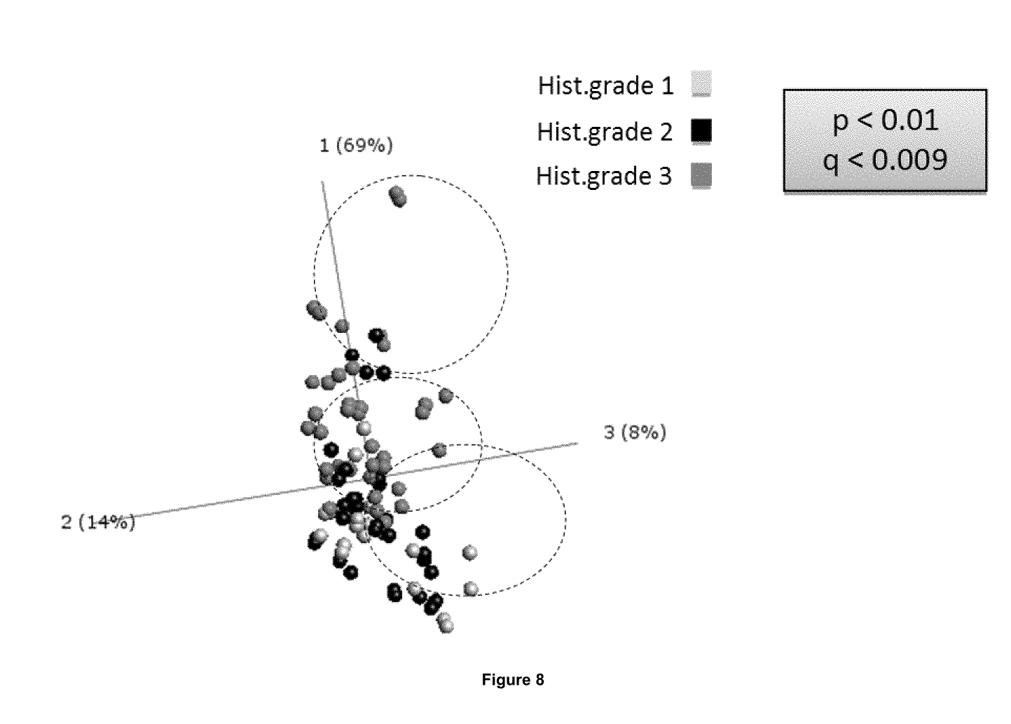

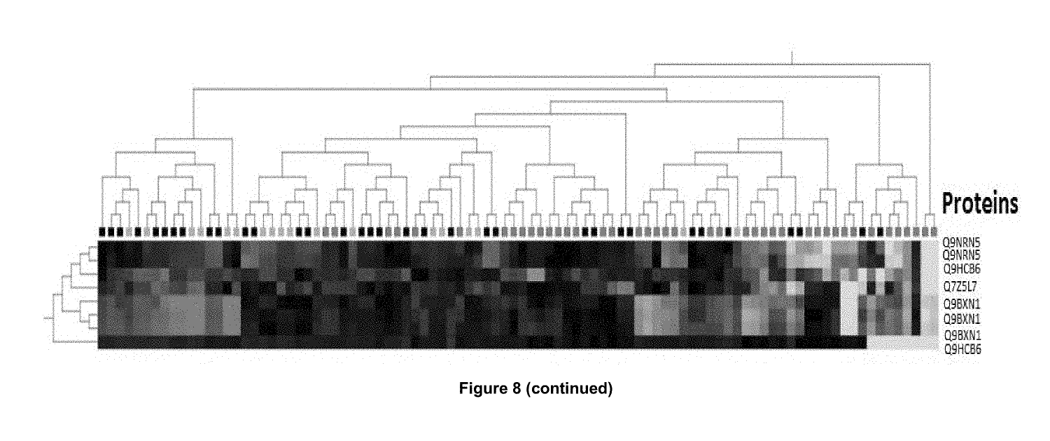

FIG. 8. Breast cancer tissue samples were selected from the same original cohort of 52 samples, here including 47 samples (with technical replicates) spread among grade 1, 2 and 3. The samples were digested (trypsinated) in gel and analysed using Selective Monitoring Reaction (SRM) set-up (an established mass spectrometry based approach). 8 peptides corresponding to 4 proteins from the stated list of biomarkers were targeted and quantified. Samples were run in duplicate. Data was analysed using Anubis followed by P-value filtering (p<0.01) and q-value filtering (q<0.009). The data showed that the breast cancer tissues samples could be differentiated according to grade, using a truncated list of markers.

### EXAMPLES

Introduction

Tumor progression and prognosis in breast cancer patients is difficult to assess using current clinical and laboratory parameters, and no candidate multiplex tissue biomarker signature exist. In an attempt to resolve this clinical unmet need, we applied a recently developed proteomic discovery tool, denoted global proteome survey. Thus, by combining affinity proteomics, based on 9 antibodies only, and label-free LC-MS/MS, we profiled 52 breast cancer tissue samples, representing one of the largest breast cancer tissue proteomic studies, and successfully generated detailed quantified proteomic maps representing 1388 proteins. The results showed that we have deciphered in-depth molecular portraits of histologic graded breast cancer tumors reflecting tumor progression. In more detail, a 49-plex tissue biomarker signature (where p<0.01) and a 79-plex tissue biomarker (where p<0.02) signature discriminating histologic grade 1 to 3 breast cancer tumors with high accuracy were defined. Highly biologically relevant proteins were identified, and the differentially expressed proteins supported the current hypothesis regarding the remodeling of the tumor microenvironment for tumor progression. In addition, using the markers to estimate the risk of distant metastasis free survival was also demonstrated. Furthermore, breast cancer associated biomarker signatures reflecting ER-, HER2-, and Ki67-statues were delineated, respectively. The biomarkers signatures were corroborated using an independent method (mRNA profiling) and patient cohort, respectively. Taken together, these molecular portraits provide improved classification and prognosis of breast cancer.

Experimental Procedures

Clinical Samples

This study was approved by the regional ethics review board in Lund, Sweden. Fifty-two breast cancer patients were recruited from the Department of Oncology (SUS, Lund). Full clinical records were accessible for 50 of the tissue samples. The samples were subdivided based on histologic grade 1 (n=9), grade 2 (n=17), and grade 3 (n=24).

Preparation of Trypsin-Digested Human Breast Cancer Tissue Samples

Proteins were extracted from 52 breast cancer tissue pieces and subsequently reduced, alkylated, trypsin digested, and finally stored at −80° C. until further use. In addition, a pooled sample, used as reference sample, was generated by combining 5 μl aliquots from all digested samples, and stored at −80° C. until further use. Details on sample preparation are provided in Supplemental Experimental Procedures.

Production and Coupling of CIMS-scFv Antibodies to Magnetic Beads

Nine CIMS scFv antibodies (Table S2) directed against six short C-terminal amino acid peptide motifs were produced in E. coli cultures, and purified using Ni²⁺-NTA affinity chromatography. Next, the purified antibodies were coupled to magnetic beads. Details on scFv production and coupling are provided in Supplemental Experimental Procedures.

Label-Free Quantitative GPS Experiments

Four different pools (denoted CIMS-binder mix 1 to 4) of antibody-conjugated beads were made by mixing equal amounts of two or three different binders (Table S2). The antibody mixes were exposed to a tryptic sample, washed, and finally incubated with acetic acid in order to elute captured peptides. The eluate was then used directly for MS-analysis without any additional clean up. The complete study was run using 26 days of MS-instrumentation time, divided into four blocks of 6.5 days (one CIMS-binder mix/block). All samples were individually analyzed one time per CIMS-binder mix. In addition, triplicate captures of selected samples were performed within each block as back-to-back LC-MS/MS runs. The reference sample was repeatedly analysed over time within and between the 4 blocks (FIG. S1). A total of 238 LC-MS/MS runs were performed and all details on the peptide capture and associated mass spectrometry analysis are provided in Supplemental Experimental Procedures.

Protein Identification and Quantification

The generated data was analyzed by two software packages, Proteios SE (Hakkinen et al., 2009) and Progenesis LC-MS (Nonlinear Dynamics, UK). Searches were performed against a forward and a reverse combined database (Homo Sapiens Swiss-Prot, August-2011, resulting in a total of 71324 database entries) with a false discovery rate (FDR) of 0.01 estimated on the basis of the number of identified reverse hits for generating peptide identifications. The Progenesis-LC-MS software (v 4.0) was used for aligning features, identification (Mascot), and generating quantitative values. Details regarding search parameters and data processing are provided in Supplemental Experimental Procedures.

Statistical and Bioinformatical Analysis

Qlucore Omics Explorer v (2.2) (Qlucore AB, Lund, Sweden) was used for identifying significantly up- or down-regulated proteins (p<0.01) using a one-way ANOVA. The q-values were generated based on the Benjamini and Hochberg method (Benjamini and Hochberg, 1995). Principal component analysis (PCA) plots and heatmaps were generated in Qlucore. The support vector machine (SVM) is a learning method (Cortes and Vapnik, 1995) that was used to classify the samples using a leave-one-out cross-validation procedure and the analyses were performed on both unfiltered and p-value filtered data. A receiver operating characteristics (ROC) curve (Lasko et al., 2005), constructed using the SVM decision values and the area under the curve (AUC), was used as a measurement of the performance of the classifier. Furthermore, the Ingenuity Systems Pathway Analysis (IPA) (v 11904312, www.ingenuity.com) was used for the significantly differentially expressed proteins in order for extracting information, such as protein localisation, potential network interactions, transcription factor associations, and association with tumorigenesis.

The experimentally derived protein signatures were finally validated at the mRNA level using the GOBO search tool (Ringner et al., 2011) against large cohorts of published gene expression data for breast cancer tissues with clinical parameters such as histologic grades 1, 2 and 3, ER-status or HER2-status.

Supplementary Experimental Procedures

Preparation of Trypsin-Digested Human Breast Cancer Tissue Samples

Protein was extracted from the breast cancer tissue pieces, and stored at −80° C. until use. Briefly, tissue pieces (about 50 mg/sample) were homogenized in Teflon containers, pre-cooled in liquid nitrogen, by fixating the bomb in a shaker for 2×30 seconds with quick cooling in liquid nitrogen in between the two shaking rounds. The homogenized tissue powder was collected in lysis buffer (2 mg tissue/30 μl buffer) containing 8 M urea, 30 mM Tris, 5 mM magnesium acetate and 4% (w/v) CHAPS (pH 8.5). The tubes were briefly vortexed and incubated on ice for 40 min, with brief vortex of the sample every 5 minutes. After incubation, the samples were centrifuged at 13000 rpm, and the supernatant was transferred to new tubes followed by a second centrifugation. The buffer was exchanged to 0.15 M HEPES, 0.5 M Urea (pH 8.0) using Zeba desalting spin columns (Pierce, Rockford, Ill., USA) before the protein concentration was determined using Total Protein Kit, Micro Lowry (Sigma, St. Louis, Mo., USA). Finally, the samples were aliquoted and stored at −80° C. until further use. The protein extracts were thawed, reduced, alkylated and trypsin digested. First, SDS and TCEP-HCl (Thermo Scientific, Rockford, Ill., USA) were added to 0.02% (w/v) and 5 mM, respectively, and the samples were reduced for 60 minutes at 56° C. The samples were cooled down to room temperature before iodoacetamide was added to 10 mM and then alkylated for 30 minutes at room temperature. Next, sequencing-grade modified trypsin (Promega, Madison, Wis., USA) was added at 20 μg per mg of protein for 16 hours at 37° C. In order to ensure complete digestion, a second aliquot of trypsin (10 μg per mg protein) was added and the tubes were incubated for an additional 3 hours at 37° C. Finally, the digested samples were aliquoted and stored at −80° C. until further use. In addition, a separate pooled sample, generated by combining 5 μl aliquots from all digested samples, was prepared and stored at −80° C. until further use. In order to increase the potential tentative proteome coverage, the two samples for which limited clinical data were at hand Table 51, were still analyzed individually as well as included in the pooled sample.

Production and Coupling of CIMS-scFv Antibodies to Magnetic Beads

Nine CIMS scFv antibodies (clones 1-B03, 15-A06, 17-008, 17-E02, 31-001-D01, 32-3A-G03, 33-3C-A09, 33-3D-F06 and 34-3A-D10 directed against six short C-terminal amino acid peptide motifs (denoted M-1, M-15, M-31, M-32, M-33, and M-34), were selected from the n-CoDeR (Soderlind et al., 2000) library, and kindly provided by BioInvent International AB, Lund, Sweden (Table S2). The specificity and dissociation constant (low μM range) for six of the CIMS antibodies have recently been determined (Olsson et al., 2011). The antibodies were produced in 100 ml E. coli cultures and purified using affinity chromatography on Ni²⁺-NTA agarose (Qiagen, Hilden, Germany). Bound molecules were eluted with 250 mM imidazole, dialyzed against PBS (pH 7.4) for 72 hours and then stored at +4° C. until use. The protein concentration was determined by measuring the absorbance at 280 nm. The integrity and purity of the scFv antibodies was confirmed by running Protein 80 chips on Agilent Bioanalyzer (Agilent, Waldbronn, Germany). The purified scFvs were individually coupled to magnetic beads (M-270 carboxylic acid-activated, Invitrogen Dynal, Oslo) as previously described (Olsson et al., 2011). Briefly, batches of 180-250 μg purified scFv was covalently coupled (EDC-NHS chemistry) to ˜9 mg (300 μl) of magnetic beads, and stored in 0.005% (v/v) Tween-20 in PBS at 4° C. until further use. In addition was a batch of blank beads generated (i.e. beads generated with the coupling protocol but without adding scFv).

Label-Free Quantitative GPS Experiments

Four different pools (denoted CIMS-binder mix 1 to 4) of conjugated beads were made by mixing equal amounts of two or three different binders according to the following: mix 1 (CIMS-33-3D-F06 and CIMS-33-3C-A09), mix 2 (CIMS-17-008 and CIMS-17-E02), mix 3 (CIMS-15-A06 and CIMS-34-3A-D10) and mix 4 (CIMS-1-B03, CIMS-32-3A-G03, and CIMS-31-001-D01) (Table S2). For each capture, 50 μl of the pooled bead solution was used and the scFv-beads were never reused. The beads were prewashed with 350 μl PBS prior to being exposed to a tryptic sample digest in a final volume of 35 μl (diluted with PBS and addition of phenylmethylsulfonyl fluoride (PMSF) to a final concentration of 1 mM) and then incubated with the beads for 20 min with gentle mixing. Next, the tubes were placed on a magnet, the supernatant removed, and the beads were washed with 100 and 90 μl PBS, respectively (the beads were transferred to new tubes in between each washing step and the total washing time was 5 min). Finally, the beads were incubated with 9.5 μl of a 5% (v/v) acetic acid solution for 2 min in order to elute captured peptides. The eluate was then used directly for mass spectrometry analysis without any additional clean up.

An ESI-LTQ-Orbitrap XL mass spectrometer (Thermo Electron, Bremen, Germany) interfaced with an Eksigent nanoLC 2DTM plus HPLC system (Eksigent technologies, Dublin, Calif., USA) was used for all samples. The auto-sampler injected 6 μl of the GPS-generated eluates. A blank LC-MS/MS run was used between each analyzed sample. Peptides were loaded with a constant flow rate of 15 μl/min onto a pre-column (PepMap 100, C18, 5 μm, 5 mm×0.3 mm, LC Packings, Amsterdam, Netherlands). The peptides were subsequently separated on a 10 μm fused silica emitter, 75 μm×16 cm (PicoTip™ Emitter, New Objective, Inc. Woburn, Mass., USA), packed in-house with Reprosil-Pur C18-AQ resin (3 μm Dr. Maisch, GmbH, Germany). Peptides were eluted with a 35 minutes linear gradient of 3 to 35% (v/v) acetonitrile in water, containing 0.1% (v/v) formic acid, with a flow rate of 300 nl/min. The LTQ-Orbitrap was operated in data-dependent mode to automatically switch between Orbitrap-MS (from m/z 400 to 2000) and LTQ-MS/MS acquisition. Four MS/MS spectra were acquired in the linear ion trap per each FT-MS scan, which was acquired at 60,000 FWHM nominal resolution settings using the lock mass option (m/z 445.120025) for internal calibration. The dynamic exclusion list was restricted to 500 entries using a repeat count of two with a repeat duration of 20 seconds and with a maximum retention period of 120 seconds. Precursor ion charge state screening was enabled to select for ions with at least two charges and rejecting ions with undetermined charge state. The normalized collision energy was set to 35%, and one micro scan was acquired for each spectrum. All samples were analyzed individually one time per CIMS-binder mix. In addition, a triplicate capture of the pooled sample (based on all samples in the study) was performed for each CIMS-binder mix and distributed for MS-analysis over a longer time period (start, middle and the end of the LC-MS sequence run order per binder mix) (FIG. S1). This was possible for CIMS-binder mix 1 and 4. However, more than halfway in the sequence runs for both CIMS-binder-mix 2 and 3 the analytical LC-column needed to be replaced (twice) and it was decided to run the scheduled last pool runs directly on the new replaced columns resulting in that a few (11 respectively 9 samples) were analyzed after the pool runs. Furthermore, triplicate captures were performed on samples (7267, 8613) for each CIMS-binder mix. Blank beads, i.e. beads without any conjugated antibody, were exposed to the pooled digest, in order to evaluate potential bead background binding peptides. Based on the low number of identified background binding peptides from two blank bead “captures”, all generated data was left unfiltered unless noted.

Protein Identification and Quantification

The generated data was first analyzed using the Proteios SE for generating identifications using both Mascot and X!Tandem. Briefly, all files were processed and converted into mzML and mgf format using the Proteios (v 2.17) platform and the following search parameters were used for Mascot and X!Tandem: enzyme: trypsin; missed cleavages 1; fixed modification: carbamidomethyl (C); variable modification: methionine oxidation (O). In addition, a variable N-acetyl was allowed for searches performed in X!Tandem (www.thegpm.org/tandem/). A peptide mass tolerance of 3 ppm and fragment mass tolerance of 0.5 Da was used and searches were performed against a forward and a reverse combined database (Homo Sapiens Swiss-Prot, August-2011, resulting in a total of 71324 database entries). The automated database searches in both Mascot and X!Tandem and consequently combination (with a false discovery rate (FDR) of 0.01) was used (estimated on the basis of the number of identified reverse hits) for generating peptide identifications. When generating protein identifications for each sample using the Proteios SE, a FDR of 0.01 on the protein level was applied. All raw data is stored within the Proteios SE.

Since the Proteios SE at the time of analysis offered no quantitative label-free plug-in analyzing modules (development in progress), the Progenesis-LC-MS software (v 4.0) was used for generating all quantitative values. Briefly, the raw data files were converted to mzXML using the ProteoWizard software package prior to using the Progenesis-LC-MS software. The built-in feature finding tool, Mascot search tool and combined fractions tool (CIMS-binder-mix 1, 2, 3 and 4) with default settings and minimal input was used. In order for optimal feature alignment, the first injection run of the pooled sample, for respectively CIMS-binder mix (FIG. S1), was used as reference alignment file, except for CIMS-mix 3 runs, where the halfway pool run was used as the reference alignment file. Features aligned and detected, between retention times 10-50 min for CIMS-binder mix 1 and 2 and between 10-49 min for CIMS-binder mix 3 and 4, were included for quantification. The generated normalized abundance values were extracted and used for statistical and bioinformatics analysis. Due to limitations with the Progenesis software, the identifications was limited to only Mascot searches, meaning that no X!Tandem generated identifications from Proteios SE were included for downstream quantitative analysis. The same database (Homo Sapiens Swiss-Prot, August-2011, a forward and a reverse combined database) and search parameters as mentioned above was used, and a cut-off FDR value of 0.01 was applied.

Results

In this study, semi-global protein expression profiles (identification and quantification) of 52 crude breast cancer tissue extracts were deciphered using GPS. Tissue biomarker signatures reflecting histologic grade, as well as other key clinical laboratory parameters, such as estrogen receptor (ER), HER2, and Ki-67 were delineated. An overall workflow outlining the experimental design is shown in Figure S1.

Protein Coverage, Dynamic Range, and Assay Performance

Using GPS, a total of 2,140 protein groups were identified (FIG. 1A-C). The identification reproducibility was high, resulting in a 54.7% peptide overlap (Figure S2A). In comparison, the reference sample, which was repeatedly analysed throughout the entire project, showed a 43.9% peptide identification overlap (Figure S2B). Of the identified proteins, a total of 1388 were successfully quantified (Figure S3), and subsequently used in the search for disease-associated markers. The total median CV value for quantification for the 7267-sample was found to be 10.8% (FIG. 2A), while the corresponding total median CV value for the reference sample was 22.8% (FIG. 2A). Notably, about 38% (833 peptides) of the quantified peptides, corresponding to 61 proteins had not previously been reported in the PeptideAtals (FIG. 1D), indicating on a substantial novel coverage. This was further highlighted by the fact that a significant portion of the detected peptides were shorter, with a median length of 9 versus 11 amino acids (FIG. 1E), than those previously reported.

The distribution of measured log₂-MS intensity normalized abundances for all quantified proteins was assessed and indicated a dynamic range of ˜10⁶ (Figure S3A). The in-depth coverage generated by the GPS assay was further illustrated by the fact that peptides, ranging from frequently reported in the PeptideAtlas to rarely reported, readily were detected (FIG. 1F). The detected proteins were then grouped by major biological processes, and found to be distributed among several groups (FIG. S3B). Interestingly, proteins grouped with processes, such as translation (e.g. 60S ribosomal protein), were, as might be expected, found to display a higher overall abundance than other proteins involved in e.g. mitosis (e.g. CDK1). Taken together, the data showed the capability of GPS to provide a novel and deep coverage in a reproducible manner.

Protein Expression Profiles Reflecting Histologic Grade

First, we examined whether a tissue biomarker signature reflecting histologic grade could be deciphered. Using a multivariate analysis (3 group comparison), 49 significantly (p<0.01, q-value <0.25) differentially expressed proteins were identified between the grade 1, grade 2, and grade 3 cohorts. Based on this signature, PCA-plots showed that histologic grade 1 and grade 3 tumors could be well separated, while histologic grade 2 tumors appeared to be more heterogeneous and were spread among both of the other groups (FIG. 3A). A pattern of both up- and down-regulated analytes with increasing histologic grade could be observed. As for example, cyclin-dependent kinase 1 (CDK1), minichromosome maintenance complex component 3 (MCM3), DNA replication licensing factor MCM7, ATP-citrate synthase (ACLY), polyadenylate-binding protein 4 (PABPC4), and 6-phosphofructokinase type C (PFKP) were among the up-regulated tissue markers (FIG. 3A and FIG. S4). In contrast, analytes such as keratocan (KERA), spondin (SPON1), asporin (ASPB), adipocyte enhancer-binding protein 1 (AEBP1), chymase (CMA1), and olfactomedin-like protein 3 (OLFML3) were among the down-regulated analytes, i.e. displayed higher expression levels in histologic grade 1 tumors (FIG. 3A and FIG. S5).

We then examined whether the 49 p-value filtered (p<0.01) biomarker list could be used to classify the tissues based on histologic grade. To this end, we ran a leave-one-out cross-validated with SVM and collected the decision values for all samples. The prediction values were then used to construct a ROC curve, and the AUC values were calculated (FIG. 3A). The results showed that the histologic grade tumor subgroups could be well separated (AUC=0.75-0.93), although grade 2 again appeared to be more heterogeneous.

Next, we investigated the impact of using a two-group comparison instead of a multivariate approach to define differentially expressed markers (FIG. S6). As might be expected, the data showed that the classification of the individual histologic subgroups improved as judged by the AUC-values (AUC=0.91-0.92). Focusing on histologic grade 1 versus grade 3, 50 significantly (p<0.01) differentially expressed analytes were delineated, of which 31 overlapped with the previous 49-biomarker signature (cfs. FIGS. 3 and S6C). When histologic grade 2 was mapped onto the frozen 50 biomarker comparison of grade 1 versus grade 1, it again displayed a heterogeneous feature and was spread among both cohorts (cfs. FIGS. 3A and S6D).

Impact of ER-Status

Since 14 of 24 histologic grade 3 tumors were classified as ER-negative and 14 of 17 ER-negative samples were in fact grade 3 tumors, we investigated the direct impact of ER-status on the expression profile. To test this hypothesis, the tumors were re-examined using the ER-positive samples (n=33) only. Adopting a multivariate approach, the results showed that 18 significantly differentially expressed proteins (p<0.01, q-value <0.51) were pin-pointed and histologic grade 1 versus grade 3 tissues could be well classified (AUC-value of 0.9, data not shown). Notably, 16 of 18 analytes (e.g. ASPN, SPON1, KERA, ACLY, APCS and PABPC4) were found to overlap with the originally deciphered 49 biomarker signatures (FIG. 3A). Hence, the data further supported the observation that the 49 biomarker signature reflected histologic grade.

In addition, we also examined whether an ER-associated tissue biomarker signature could be unraveled. The results showed that ER-positive and ER-negative breast cancer tissues could be well classified (AUC=0.82) (FIG. 3B), and that 39 differentially expressed analytes (p<0.01, q-value <0.32) were identified (e.g. GREB1) FIGS. 3B and 57A). Hence, the data showed that an ER-associated tissue biomarker signature had been detected.

Protein Expression Profiles Reflecting HER2/Neu-Status and Ki67-Status

When comparing the 52 breast cancer tissue extracts based on HER2/neu-status using a leave-one-out cross-validation, the data showed that the 2 cohorts could be discriminated (AUC=0.98) and that five differentially expressed markers (p<0.01, q-value <0.9) were identified (FIG. 3C). Most importantly, the receptor tyrosine-protein kinase erbB-2 (HER2) was found to be among the up-regulated proteins (FIG. 3C and FIG. S7B).

Furthermore, in a similar manner, a tissue protein signature reflecting Ki67-status (where 25% of Ki67-positive cancer nuclei was used as cut-off) could also be deciphered. In total, 45 proteins were found to be differentially expressed (p<0.01, q-value <0.27) (FIG. S8A). The data demonstrated that Ki67-positive versus Ki67-negative tumors could be separated (AUC=0.84) (FIG. S8B). Hence, the results showed that protein expression profiles reflecting both HER2/neu status and Ki67-status had been pin-pointed.

Biological Relevance

The biological relevance of the 49 tissue biomarker signature differentiating histologic grade 1 to 3 was then examined. To this end, the cellular localization of each individual protein was mapped using the IPA software (FIG. 4A), and network associated functions and potential relationships were investigated (FIG. 4B-4C). A pattern of mainly down-regulated proteins (extra cellular matric (ECM)) and up-regulated analytes (plasma membrane, cytoplasm, and nucleus) reflecting cellular localization was revealed. More importantly, the top ranked network was found to be associated with DNA replication, recombination, and repair, cell cycle and free radical scavenging, while the second highest ranked network was associated with gene expression, infectious disease and cancer. Noteworthy, several of the proteins within the top 1 network were directly or indirectly associated with NF-kB and VEGF (FIG. 4B). Furthermore, a majority of the ECM proteins were pin-pointed within the second network, and several were directly or indirectly associated with transforming growth factor-β (TGFβ1) (FIG. 4C). Hence, the results showed that biologically highly relevant tissue biomarkers reflecting histologic grade had been identified

In addition, the relationship between the 49 tissue biomarker signature and transcription factor network was also assessed using IPA (FIG. S9). Of note, Rb and E2F2 were found to be among the top associated transcription regulators (Figure S9A). In comparison, the estrogen receptor 2 (ESR2) and progesterone receptor (PGR) were found to be among the top associated regulators when the tissue biomarker signature differentiating ER-positive versus ER-negative tumors (Figure S9B).

Validation of Candidate Breast Cancer Progression Signature

In an attempt to validate the 49 tissue biomarker signature discriminating histologic grade 1 to 3, the data was compared to publicly available orthogonal breast cancer mRNA profiling data set. The validation cohort was composed of 1,881 samples, of which 1,411 with assigned histologic grade, including grade 1 (n=239), grade 2 (n=677), and grade 3 (n=495). Forty-two of 49 tissue biomarkers could be mapped to the gene expression data base using gene entrez ID, and were subsequently used in the validation test.

The 42 tissue markers were then split into two groups, based on the observed down-(15 analytes) or up-regulated (27 analytes) protein expression profile for grade 3 versus grade 1, and compared to the corresponding mRNA expression profiles (FIG. 5). The protein expression profiles of both down-regulated (e.g. SPON1 and KERA) (FIGS. 5A, S5, S10I, and S11J) and up-regulated proteins (e.g. CDK1 and MCM3) (FIGS. 5B, S4, S10A, and S10B) were found to corroborate well with the mRNA expression levels. Interestingly, the up-regulated markers were found to display mRNA profiles with a high correlation to checkpoint and M-phase gene modules (FIG. 5A), while the group of down-regulated markers displayed mRNA profiles with high correlation to the stroma gene set module (FIG. 5B).

Validation of ER- and HER2-Associated Tissue Biomarker Signatures

In a similar manner, attempts were then made to validate the tissue biomarker signatures reflecting ER-status (FIG. 3B), and HER2-status (FIG. 3C), using the same publicly available orthogonal breast cancer mRNA profiling data set as above.

In case of ER, the validation set was composed of 1,620 samples with assigned ER-status, including 395 ER-negative and 1225 ER-negative samples. Thirty-two of 39 tissue biomarkers could be mapped to the gene expression data base, and were subsequently used in the validation. The 32 markers were then split into two groups (10 up-regulated and 22 down-regulated) based on the observed protein expression profile, and compared to the corresponding mRNA expression profiles (FIG. S11). With a few exceptions (e.g. complement C3 (FIG. S11F and FIG. S7A), the observed protein expression profiles corroborated well with the corresponding mRNA expression profiles (cfs. FIGS. 3B, S7A, and S11). In this context, it was of interest to note that the group of up-regulated proteins in ER-positive tumors were found to display mRNA profiles with high correlation to the steroid response gene module, while the group of down-regulated proteins were found to display mRNA profiles with high correlation to the immune-response and basal gene set modules (FIG. S11A-S11B).

The validation set for HER2 was composed of 1,881 samples, split into HER2-positive (n=152), basal (n=357), luminal-A (n=483), luminal-B (n=289), normal like (n=257), and unclassified (n=344). Three of 5 tissue markers could be mapped to the validation data set, and was used in the subsequent evaluation (FIG. S12). The results showed that the protein expression profiles and gene expression profiles correlated well (cfs. FIGS. 3C, S7B and S12), further validating the observations.

Assessing Distant Metastasis Free Survival

Finally, we examined whether the 49 tissue biomarker signature reflecting histologic grade also could be used to assess the risk of distant metastasis free survival (DMFS) again using the same publicly available gene expression data set. Forty-two of 49 tissue biomarkers could be mapped to 1379 samples with 10-year endpoint survival data. The markers were split in two groups, reflecting down-regulated (n=15)) and up-regulated (n=27) markers in grade 3 versus grade 1, and Kaplan-Meier analysis were then performed with DMFS with a 10-year endpoint by stratifying the gene expression data into three quantiles (low, intermediate, and high) based on the expression levels of these analytes (FIG. S13). The data showed that in particular the cohort of down-regulated analytes (mainly ECM-associated analytes) predicted the risk of DMFS. In fact, this could be accomplished by targeting single down-regulated (e.g. KERA and OLFM3) or up-regulated (e.g. CDK1) biomarkers.

### DISCUSSION

In this study we have deciphered the first in-depth, multiplexed tissue biomarker signature reflecting tumor progression in breast cancer, taking the next step towards personalized medicine in breast cancer. This achievement was accomplished using our recently in-house developed GPS technology (Olsson et al., 2012; Olsson et al., 2011; Wingren et al., 2009). Hence, by combining affinity proteomics, based on 9 antibodies only, and label-free LC-MS/MS, we profiled 52 breast cancer tissue samples, representing one of the largest breast cancer tissue proteomic studies, and successfully generated detailed quantified proteomic maps reflecting 1388 proteins.

In more detail, the first 49-plex tissue biomarker signature differentiating histologic grade 1 to 3 breast cancer tumors with high specificity and sensitivity was delineated. This list can be extended to 79 differentially expressed markers setting the p-value criteria to p<0.02, but here the discussions focussed towards the top 49 analytes (p<0.01). The molecular profile, or protein fingerprints, supported the current view that grade 1 and grade 3 tumors were more distinct, while grade 2 tumors were more heterogeneous (Sotiriou et al., 2006). When dissecting the signature a priori known markers, known to be associated with breast cancer, as well as novel candidate biomarkers were identified. From a technical point of view, this novel coverage was reflected by the fact that a large portion (˜38%) of the quantified peptides had not be previously been reported in the PeptideAtlas database (Deutsch et al., 2008). This novel coverage provided by the GPS set-up also became evident when searching for these 49 analytes against the Human Protein Atlas project (Uhlen et al., 2010). Although the Human Protein Atlas project currently covers more than 50% of the non-redundant human proteome, had neither any antibodies nor any histology staining reported for 13 of 49 differentially expressed proteins.

ER-status alone has been shown to affect the expression of more than 10% of the genes in breast tumors, and is generally thought to have an impact on survival. Since ER-negative breast cancers generally are more aggressive and anti-estrogen based therapy is inefficient, additional targeted therapies are urgently needed (Rochefort et al., 2003). We identified a 39 protein signature capable of differentiating ER-positive and ER-negative tumors with adequate specificity and sensitivity. Noteworthy, 11 of 39 markers have not yet been covered by the Human Protein Atlas project, again outlining the novel coverage provided by the GPS technology (Uhlen et al., 2010). One of the 39 markers, GREB1, has been suggested as a candidate clinical marker for response to endocrine therapy as well as a potential therapeutic target (Hnatyszyn et al., 2010; Rae et al., 2005). GREB1 is an estrogen-regulated gene that mediates estrogen-stimulated cell proliferation and was recently reported to be expressed in ER-positive breast cancer cells and normal breast tissue, but not in ER-negative samples outlining its potential as surrogate marker for ER (Hnatyszyn et al., 2010). The protein profile generated with GPS further supported this notion (Figure S7A).

Furthermore, a 5 protein signature capable of discriminating the clinically defined HER2-positive and HER2-negative samples was deciphered (FIG. 3C). In fact, the low abundant receptor tyrosine-protein kinase erbB-2 (HER2-protein) was identified, quantified and found to be one of the differentially expressed markers. Hence, the potential of measuring HER2 using GPS in clinical settings could be envisioned as a complement to currently used classical immunohistochemistry or fluorescence in situ hybridization (FISH) based detection systems. A recent study indicated that one in five HER2-based tests might generate incorrect results (Phillips et al., 2009). In addition, S100-A9 and the growth factor receptor-bound protein 7 (GRB7) were also found to display an increased expression in a majority of HER2-positive defined samples (FIG. S7B). High GRB7 expression was recently reported to be associated with high HER2-expression, and used to define a subset of breast cancer patients with decreased survival (Nadler et al., 2010). The S100 gene family encode for low molecular weight calcium-binding proteins, and specific S100 members have been associated with cancer progression, metastasis, and to have a potential as a prediction marker of drug resistance in patients with breast cancer (McKiernan et al., 2011; Yang et al., 2011).

Most importantly, not only the biomarker signature reflecting histologic grade, but also those reflecting ER-status and HER2-status, were validated using an independent data set and an orthogonal method (mRNA expression levels) using the GOBO-tool (Ringner et al., 2011). Groups of up- and down-regulated proteins were evaluated based on correlation to known gene set modules, since it often is the functional processes captured by a gene signature, and not the individual genes that are important (Wirapati et al., 2008). The significant correlation to the gene-set modules for stroma, checkpoint, and steroid responses were in particular noteworthy (FIG. 5 and FIG. S11). Furthermore, when assessing the DMFS as endpoint, using the histologic derived protein analytes, the data clearly indicated worse clinical outcome, in particular when using the down-regulated ECM proteins. Hence, the independent mRNA validations, added strong support for reported candidate biomarker signatures and their potential in future breast tissue tumor classifications.

Taken together, we have demonstrated the applicability of our recently developed GPS technology platform for clinical proteomic discovery profiling efforts. Tissue biomarker signatures reflecting histologic grade, i.e. tumor progression, as well as other key clinical laboratory parameters, such as ER-, HER2-, and Ki67-status have been reported in this study; these novel tissue biomarker portraits allow for improved classification and prognosis of breast cancer.

### REFERENCES

Aebersold, R., and Mann, M. (2003). Mass spectrometry-based proteomics. Nature 422, 198-207.
Benjamini, Y., and Hochberg, Y. (1995). Controlling the false discovery rate: a practical and powerful approach to multiple testing. Journal of the Royal Statistical Society Series B 57, 289-300.
Bergamaschi, A., Tagliabue, E., Sorlie, T., Naume, B., Triulzi, T., Orlandi, R., Russnes, H. G., Nesland, J. M., Tammi, R., Auvinen, P., et al. (2008). Extracellular matrix signature identifies breast cancer subgroups with different clinical outcome. The Journal of pathology 214, 357-367.
Bierie, B., and Moses, H. L. (2006). Tumour microenvironment: TGFbeta: the molecular Jekyll and Hyde of cancer. Nature reviews Cancer 6, 506-520.
Borrebaeck, C. A., and Wingren, C. (2011). Recombinant antibodies for the generation of antibody arrays. Methods Mol Biol 785, 247-262.
Bouchal, P., Roumeliotis, T., Hrstka, R., Nenutil, R., Vojtesek, B., and Garbis, S. D. (2009). Biomarker discovery in low-grade breast cancer using isobaric stable isotope tags and two-dimensional liquid chromatography-tandem mass spectrometry (iTRAQ-2DLC-MS/MS) based quantitative proteomic analysis. Journal of proteome research 8, 362-373.
Carlsson, A., Wingren, C., Ingvarsson, J., Ellmark, P., Baldertorp, B., Ferno, M., Olsson, H., and Borrebaeck, C. A. (2008). Serum proteome profiling of metastatic breast cancer using recombinant antibody microarrays. Eur J Cancer 44, 472-480.
Carlsson, A., Wingren, C., Kristensson, M., Rose, C., Ferno, M., Olsson, H., Jernstrom, H., Ek, S., Gustaysson, E., Ingvar, C., et al. (2011). Molecular serum portraits in patients with primary breast cancer predict the development of distant metastases. Proceedings of the National Academy of Sciences of the United States of America 108, 14252-14257.
Ciocca, D. R., and Elledge, R. (2000). Molecular markers for predicting response to tamoxifen in breast cancer patients. Endocrine 13, 1-10.
Cortes, C., and Vapnik, V. (1995). Support-Vector Networks. Machine Learning 20, 273-297.
Cortez, D., Glick, G., and Elledge, S. J. (2004). Minichromosome maintenance proteins are direct targets of the ATM and ATR checkpoint kinases. Proceedings of the National Academy of Sciences of the United States of America 101, 10078-10083.
Desmedt, C., Haibe-Kains, B., Wirapati, P., Buyse, M., Larsimont, D., Bontempi, G., Delorenzi, M., Piccart, M., and Sotiriou, C. (2008). Biological processes associated with breast cancer clinical outcome depend on the molecular subtypes. Clinical cancer research: an official journal of the American Association for Cancer Research 14, 5158-5165.
Deutsch, E. W., Lam, H., and Aebersold, R. (2008). PeptideAtlas: a resource for target selection for emerging targeted proteomics workflows. EMBO reports 9, 429-434.
Dowsett, M., Goldhirsch, A., Hayes, D. F., Senn, H. J., Wood, W., and Viale, G. (2007). International Web-based consultation on priorities for translational breast cancer research. Breast cancer research: BCR 9, R81.
Elston, C. W., and Ellis, I. O. (1991). Pathological prognostic factors in breast cancer. I. The value of histological grade in breast cancer: experience from a large study with long-term follow-up. Histopathology 19, 403-410.
Fata, J. E., Werb, Z., and Bissell, M. J. (2004). Regulation of mammary gland branching morphogenesis by the extracellular matrix and its remodeling enzymes. Breast cancer research: BCR 6, 1-11.
Frierson, H. F., Jr., Wolber, R. A., Berean, K. W., Franquemont, D. W., Gaffey, M. J., Boyd, J. C., and Wilbur, D. C. (1995). Interobserver reproducibility of the Nottingham modification of the Bloom and Richardson histologic grading scheme for infiltrating ductal carcinoma. American journal of clinical pathology 103, 195-198.
Geiger, T., Cox, J., Ostasiewicz, P., Wisniewski, J. R., and Mann, M. (2010). Super-SILAC mix for quantitative proteomics of human tumor tissue. Nature methods 7, 383-385.
Geiger, T., Madden, S. F., Gallagher, W. M., Cox, J., and Mann, M. (2012). Proteomic portrait of human breast cancer progression identifies novel prognostic markers. Cancer research.
Gong, Y., Wang, N., Wu, F., Cass, C. E., Damaraju, S., Mackey, J. R., and Li, L. (2008). Proteome profile of human breast cancer tissue generated by LC-ESI-MS/MS combined with sequential protein precipitation and solubilization. Journal of proteome research 7, 3583-3590.
Ha, S. A., Shin, S. M., Namkoong, H., Lee, H., Cho, G. W., Hur, S. Y., Kim, T. E., and Kim, J. W. (2004). Cancer-associated expression of minichromosome maintenance 3 gene in several human cancers and its involvement in tumorigenesis. Clinical cancer research: an official journal of the American Association for Cancer Research 10, 8386-8395.
Hakkinen, J., Vincic, G., Mansson, O., Warell, K., and Levander, F. (2009). The proteios software environment: an extensible multiuser platform for management and analysis of proteomics data. Journal of proteome research 8, 3037-3043.
Hanahan, D., and Weinberg, R. A. (2000). The hallmarks of cancer. Cell 100, 57-70.
Hanahan, D., and Weinberg, R. A. (2011). Hallmarks of cancer: the next generation. Cell 144, 646-674.
Hanash, S. (2003). Disease proteomics. Nature 422, 226-232.
Hnatyszyn, H. J., Liu, M., Hilger, A., Herbert, L., Gomez-Fernandez, C. R., Jorda, M., Thomas, D., Rae, J. M., El-Ashry, D., and Lippman, M. E. (2010). Correlation of GREB1 mRNA with protein expression in breast cancer: validation of a novel GREB1 monoclonal antibody. Breast cancer research and treatment 122, 371-380.
Hondermarck, H., Tastet, C., El Yazidi-Belkoura, I., Toillon, R. A., and Le Bourhis, X. (2008). Proteomics of breast cancer: the quest for markers and therapeutic targets. Journal of proteome research 7, 1403-1411.
Hudis, C. A. (2007). Trastuzumab—mechanism of action and use in clinical practice. The New England journal of medicine 357, 39-51.
Ivshina, A. V., George, J., Senko, O., Mow, B., Putti, T. C., Smeds, J., Lindahl, T., Pawitan, Y., Hall, P., Nordgren, H., et al. (2006). Genetic reclassification of histologic grade delineates new clinical subtypes of breast cancer. Cancer research 66, 10292-10301.
Jemal, A., Bray, F., Center, M. M., Ferlay, J., Ward, E., and Forman, D. (2011). Global cancer statistics. CA: a cancer journal for clinicians 61, 69-90.
Johnson, N., Li, Y. C., Walton, Z. E., Cheng, K. A., Li, D., Rodig, S. J., Moreau, L. A., Unitt, C., Bronson, R. T., Thomas, H. D., et al. (2011). Compromised CDK1 activity sensitizes BRCA-proficient cancers to PARP inhibition. Nature medicine 17, 875-882.
Kang, S., Kim, M. J., An, H., Kim, B. G., Choi, Y. P., Kang, K. S., Gao, M. Q., Park, H., Na, H. J., Kim, H. K., et al. (2010). Proteomic molecular portrait of interface zone in breast cancer. Journal of proteome research 9, 5638-5645.
Kuhn, U., and Wahle, E. (2004). Structure and function of poly(A) binding proteins. Biochimica et biophysica acta 1678, 67-84.
Lasko, T. A., Bhagwat, J. G., Zou, K. H., and Ohno-Machado, L. (2005). The use of receiver operating characteristic curves in biomedical informatics. Journal of biomedical informatics 38, 404-415.
Malumbres, M., and Barbacid, M. (2009). Cell cycle, CDKs and cancer: a changing paradigm. Nature reviews Cancer 9, 153-166.
Mangus, D. A., Evans, M. C., and Jacobson, A. (2003). Poly(A)-binding proteins: multifunctional scaffolds for the post-transcriptional control of gene expression. Genome biology 4, 223.
McKiernan, E., McDermott, E. W., Evoy, D., Crown, J., and Duffy, M. J. (2011). The role of S100 genes in breast cancer progression. Tumour biology: the journal of the International Society for Oncodevelopmental Biology and Medicine 32, 441-450.
Moon, J. S., Kim, H. E., Koh, E., Park, S. H., Jin, W. J., Park, B. W., Park, S. W., and Kim, K. S. (2011). Kruppel-like factor 4 (KLF4) activates the transcription of the gene for the platelet isoform of phosphofructokinase (PFKP) in breast cancer. The Journal of biological chemistry 286, 23808-23816.
Nadler, Y., Gonzalez, A. M., Camp, R. L., Rimm, D. L., Kluger, H. M., and Kluger, Y. (2010). Growth factor receptor-bound protein-7 (Grb7) as a prognostic marker and therapeutic target in breast cancer. Annals of oncology: official journal of the European Society for Medical Oncology/ESMO 21, 466-473.
Olsson, N., James, P., Borrebaeck, C. A., and Wingren, C. (2012). Quantitative proteomics targeting classes of motif-containing peptides using immunoaffinity-based mass spectrometry. Submitted.
Olsson, N., Wingren, C., Mattsson, M., James, P., D, O. C., Nilsson, F., Cahill, D. J., and Borrebaeck, C. A. (2011). Proteomic analysis and discovery using affinity proteomics and mass spectrometry. Molecular & cellular proteomics: MCP 10, M110 003962.
Olsson, N., Wingren, C., Mattsson, M., James, P., D, O. C., Nilsson, F., Cahill, D. J., and Borrebaeck, C. A. (2011). Proteomic analysis and discovery using affinity proteomics and mass spectrometry. Molecular & cellular proteomics: MCP 10, M110 003962.
Paik, S., Shak, S., Tang, G., Kim, C., Baker, J., Cronin, M., Baehner, F. L., Walker, M. G., Watson, D., Park, T., et al. (2004). A multigene assay to predict recurrence of tamoxifen-treated, node-negative breast cancer. The New England journal of medicine 351, 2817-2826.
Pavlidis, P., and Noble, W. S. (2003). Matrix2png: a utility for visualizing matrix data. Bioinformatics 19, 295-296.
Pei, D. S., Qian, G. W., Tian, H., Mou, J., Li, W., and Zheng, J. N. (2012). Analysis of human Ki-67 gene promoter and identification of the Sp1 binding sites for Ki-67 transcription. Tumour biology: the journal of the International Society for Oncodevelopmental Biology and Medicine 33, 257-266.
Perou, C. M., Sorlie, T., Eisen, M. B., van de Rijn, M., Jeffrey, S. S., Rees, C. A., Pollack, J. R., Ross, D. T., Johnsen, H., Akslen, L. A., et al. (2000). Molecular portraits of human breast tumours. Nature 406, 747-752.
Phillips, K. A., Marshall, D. A., Haas, J. S., Elkin, E. B., Liang, S. Y., Hassett, M. J., Ferrusi, I., Brock, J. E., and Van Bebber, S. L. (2009). Clinical practice patterns and cost effectiveness of human epidermal growth receptor 2 testing strategies in breast cancer patients. Cancer 115, 5166-5174.
Place, A. E., Jin Huh, S., and Polyak, K. (2011). The microenvironment in breast cancer progression: biology and implications for treatment. Breast cancer research: BCR 13, 227.
Rae, J. M., Johnson, M. D., Scheys, J. O., Cordero, K. E., Larios, J. M., and Lippman, M. E. (2005). GREB 1 is a critical regulator of hormone dependent breast cancer growth. Breast cancer research and treatment 92, 141-149.
Ringner, M., Fredlund, E., Hakkinen, J., Borg, A., and Staaf, J. (2011). GOBO: gene expression-based outcome for breast cancer online. PloS one 6, e17911.
Robbins, P., Pinder, S., de Klerk, N., Dawkins, H., Harvey, J., Sterrett, G., Ellis, I., and Elston, C. (1995). Histological grading of breast carcinomas: a study of interobserver agreement. Human pathology 26, 873-879.
Rochefort, H., Glondu, M., Sahla, M. E., Platet, N., and Garcia, M. (2003). How to target estrogen receptor-negative breast cancer? Endocrine-related cancer 10, 261-266.
Slamon, D. J., Leyland-Jones, B., Shak, S., Fuchs, H., Paton, V., Bajamonde, A., Fleming, T., Eiermann, W., Wolter, J., Pegram, M., et al. (2001). Use of chemotherapy plus a monoclonal antibody against HER2 for metastatic breast cancer that overexpresses HER2. The New England journal of medicine 344, 783-792.
Soderlind, E., Strandberg, L., Jirholt, P., Kobayashi, N., Alexeiva, V., Aberg, A. M., Nilsson, A., Jansson, B., Ohlin, M., Wingren, C., et al. (2000). Recombining germline-derived CDR sequences for creating diverse single-framework antibody libraries. Nature biotechnology 18, 852-856.
Sorlie, T., Perou, C. M., Tibshirani, R., Aas, T., Geisler, S., Johnsen, H., Hastie, T., Eisen, M. B., van de Rijn, M., Jeffrey, S. S., et al. (2001). Gene expression patterns of breast carcinomas distinguish tumor subclasses with clinical implications. Proceedings of the National Academy of Sciences of the United States of America 98, 10869-10874.
Sotiriou, C., Wirapati, P., Loi, S., Harris, A., Fox, S., Smeds, J., Nordgren, H., Farmer, P., Praz, V., Haibe-Kains, B., et al. (2006). Gene expression profiling in breast cancer: understanding the molecular basis of histologic grade to improve prognosis. Journal of the National Cancer Institute 98, 262-272.
Strande, V., Canelle, L., Tastet, C., Burlet-Schiltz, O., Monsarrat, B., and Hondermarck, H. (2009). The proteome of the human breast cancer cell line MDA-MB-231: Analysis by LTQ-Orbitrap mass spectrometry. Proteomics Clinical applications 3, 41-50.
Sutton, C. W., Rustogi, N., Gurkan, C., Scally, A., Loizidou, M. A., Hadjisavvas, A., and Kyriacou, K. (2010). Quantitative proteomic profiling of matched normal and tumor breast tissues. Journal of proteome research 9, 3891-3902.
Turashvili, G., Bouchal, J., Baumforth, K., Wei, W., Dziechciarkova, M., Ehrmann, J., Klein, J., Fridman, E., Skarda, J., Srovnal, J., et al. (2007). Novel markers for differentiation of lobular and ductal invasive breast carcinomas by laser microdissection and microarray analysis. BMC cancer 7, 55.
Uhlen, M., Oksvold, P., Fagerberg, L., Lundberg, E., Jonasson, K., Forsberg, M., Zwahlen, M., Kampf, C., Wester, K., Hober, S., et al. (2010). Towards a knowledge-based Human Protein Atlas. Nature biotechnology 28, 1248-1250.
van de Vijver, M. J., He, Y. D., van′t Veer, L. J., Dai, H., Hart, A. A., Voskuil, D. W., Schreiber, G. J., Peterse, J. L., Roberts, C., Marton, M. J., et al. (2002). A gene-expression signature as a predictor of survival in breast cancer. The New England journal of medicine 347, 1999-2009.
van 't Veer, L. J., Dai, H., van de Vijver, M. J., He, Y. D., Hart, A. A., Mao, M., Peterse, H. L., van der Kooy, K., Marton, M. J., Witteveen, A. T., et al. (2002). Gene expression profiling predicts clinical outcome of breast cancer. Nature 415, 530-536.
Wang, T. H., Chao, A., Tsai, C. L., Chang, C. L., Chen, S. H., Lee, Y. S., Chen, J. K., Lin, Y. J., Chang, P. Y., Wang, C. J., et al. (2010). Stress-induced phosphoprotein 1 as a secreted biomarker for human ovarian cancer promotes cancer cell proliferation. Molecular & cellular proteomics: MCP 9, 1873-1884.
Wingren, C., James, P., and Borrebaeck, C. A. (2009). Strategy for surveying the proteome using affinity proteomics and mass spectrometry. Proteomics 9, 1511-1517.
Wirapati, P., Sotiriou, C., Kunkel, S., Farmer, P., Pradervand, S., Haibe-Kains, B., Desmedt, C., Ignatiadis, M., Sengstag, T., Schutz, F., et al. (2008). Meta-analysis of gene expression profiles in breast cancer: toward a unified understanding of breast cancer subtyping and prognosis signatures. Breast cancer research: BCR 10, R65.
Yang, W. S., Moon, H. G., Kim, H. S., Choi, E. J., Yu, M. H., Noh, D. Y., and Lee, C. (2011). Proteomic Approach Reveals FKBP4 and S100A9 as Potential Prediction Markers of Therapeutic Response to Neoadjuvant Chemotherapy in Patients with Breast Cancer. Journal of proteome research.
Zhang, L., Reidy, S. P., Bogachev, O., Hall, B. K., Majdalawieh, A., and Ro, H. S. (2011). Lactation defect with impaired secretory activation in AEBP1-null mice. PloS one 6, e27795.

TABLE 1
BIOMARKERS FOR DETERMINING A BREAST CANCER-ASSOCIATED DISEASE STATE

| | A) Core biomarkers (H-grade and DMSF) | | | | | | | Up- or down regulation in individual comparisons | | | |
|---|---|---|---|---|---|---|---|---|---|---|---|
| | Prot acc. | p-Value | q-Value | F-statistic | Name | H-grade | DMSF | H1 vs H2 | H1 vs H3 | H2 vs H3 | DMSF* |
| 1 | O60938 | 3.54E−06 | 0.001483776 | 16.58840179 | KERA | yes | yes | Down H2 | Down H3 | Down H3 | Down H3 |
| 2 | Q9HCB6 | 3.27E−06 | 0.001483776 | 16.72493744 | SPON1 | yes | yes | Down H2 | Down H3 | Down H3 | Down H3 |

| | B) Preferred biomarkers (H-grade and DMSF) | | | | | | | Up- or down regulation in individual comparisons | | | |
|---|---|---|---|---|---|---|---|---|---|---|---|
| | Prot acc. | p-Value | q-Value | F-statistic | Name | H-grade | DMSF | H1 vs H2 | H1 vs H3 | H2 vs H3 | DMFS |
| 3 | P02743 | 1.74E−06 | 0.001483776 | 17.82355118 | APCS | yes | yes | Down H2 | Down H3 | Down H3 | Down H3 |
| 4 | O75348 | 0.000799949 | 0.123381185 | 8.331157684 | ATP6V1G1 | yes | yes | Down H2 | Up H3 | Up H3 | Down H3 |
| 5 | Q71UM5 | 0.00098264 | 0.123381185 | 8.053757668 | RPS27L | yes | yes | Up H2 | Up H3 | Up H3 | Up H3 |
| 6 | Q14195 | 0.001159707 | 0.123381185 | 7.832079411 | DPYSL3 | yes | yes | Down H2 | Down H3 | Down H3 | Down H3 |
| 7 | Q9BS26 | 0.001177863 | 0.123381185 | 7.811373711 | ERP44 | yes | yes | Down H2 | Up H3 | Up H3 | Up H3 |
| 8 | Q13905 | 0.001910644 | 0.184744553 | 7.173429966 | RAPGEF1 | yes | yes | Down H2 | Up H3 | Up H3 | Up H3 |
| 9 | P53396 | 0.002351787 | 0.194665471 | 6.903478146 | ACLY | yes | yes | Up H2 | Up H3 | Up H3 | Up H3 |
| 10 | P23946 | 0.002436705 | 0.194665471 | 6.857621193 | CMA1 | yes | yes | Down H2 | Down H3 | Down H3 | Down H3 |
| 11 | P25205 | 0.002640618 | 0.194665471 | 6.75398016 | MCM3 | yes | yes | Up H2 | Up H3 | Up H3 | Up H3 |
| 12 | Q9UKU9 | 0.002787572 | 0.194665471 | 6.684337139 | ANGPTL2 | yes | yes | Down H2 | Down H3 | Down H3 | Down H3 |
| 13 | Q8IUX7 | 0.00329872 | 0.210392612 | 6.468857288 | AEBP1 | yes | yes | Down H2 | Down H3 | Down H3 | Down H3 |
| 14 | Q15819 | 0.003347536 | 0.210392612 | 6.450129509 | UBE2V2 | yes | yes | Up H2 | Up H3 | Down H3 | Up H3 |
| 15 | Q6P0N0 | 0.003670423 | 0.215540903 | 6.333002567 | MIS18BP1 | yes | yes | Up H2 | Up H3 | Up H3 | Up H3 |
| 16 | Q9UBD9 | 0.003821762 | 0.215540903 | 6.281753063 | CLCF1 | yes | yes | Up H2 | Up H3 | Up H3 | Up H3 |
| 17 | P80404 | 0.004283228 | 0.220097415 | 6.137635708 | ABAT | yes | yes | Up H2 | Down H3 | Down H3 | Down H3 |
| 18 | P05141 | 0.004800725 | 0.220097415 | 5.994134426 | SLC25A5 | yes | yes | Up H2 | Up H3 | Up H3 | Up H3 |
| 19 | P31948 | 0.005012118 | 0.220097415 | 5.940101147 | STIP1 | yes | yes | Up H2 | Up H3 | Up H3 | Up H3 |
| 20 | Q9NRN5 | 0.00549968 | 0.220097415 | 5.824034214 | OLFML3 | yes | yes | Down H2 | Down H3 | Down H3 | Down H3 |
| 21 | P09693 | 0.006353439 | 0.220097415 | 5.644515991 | CD3G | yes | yes | Up H2 | Up H3 | Up H3 | Up H3 |
| 22 | P33993 | 0.006506666 | 0.220097415 | 5.614975929 | MCM7 | yes | yes | Up H2 | Up H3 | Up H3 | Up H3 |
| 23 | Q02978 | 0.006755395 | 0.220097415 | 5.568535328 | SLC25A11 | yes | yes | Down H2 | Up H3 | Up H3 | Up H3 |
| 24 | O00567 | 0.006943766 | 0.220097415 | 5.534535408 | NOP56 | yes | yes | Up H2 | Up H3 | Up H3 | Up H3 |
| 25 | O43159 | 0.006985712 | 0.220097415 | 5.527095318 | RRP8 | yes | yes | Up H2 | Up H3 | Down H3 | Up H3 |
| 26 | Q9NWH9 | 0.007683607 | 0.220097415 | 5.409715176 | SLTM | yes | yes | Up H2 | Up H3 | Up H3 | Up H3 |
| 27 | Q15631 | 0.007749403 | 0.220097415 | 5.399227619 | TSN | yes | yes | Up H2 | Up H3 | Up H3 | Up H3 |
| 28 | Q13011 | 0.007879382 | 0.220097415 | 5.378779411 | ECH1 | yes | yes | Down H2 | Up H3 | Up H3 | Up H3 |
| 29 | P51888 | 0.008461666 | 0.229086405 | 5.291296959 | PRELP | yes | yes | Down H2 | Down H3 | Down H3 | Down H3 |
| 30 | P49591 | 0.008565681 | 0.229086405 | 5.276332378 | SARS | yes | yes | Up H2 | Up H3 | Down H3 | Up H3 |
| 31 | P62851 | 0.009544854 | 0.249955868 | 5.144096375 | RPS25 | yes | yes | Up H2 | Up H3 | Up H3 | Up H3 |
| 32 | Q9BSJ8 | 0.009871082 | 0.253223467 | 5.103161812 | ESYT1 | yes | yes | Down H2 | Up H3 | Up H3 | Up H3 |

| | C) Preferred biomarkers (H-grade) | | | | | | | Up- or down regulation in individual comparisons | | | |
|---|---|---|---|---|---|---|---|---|---|---|---|
| | Prot acc. | p-Value | q-Value | F-statistic | Name | H-grade | DMSF | H1 vs H2 | H1 vs H3 | H2 vs H3 | DMSF* |
| 33 | Q7Z5L7 | 0.000518761 | 0.123381185 | 8.92324543 | PODN | yes | T.B.D. | Up H2 | Down H3 | Down H3 | T.B.D. |
| 34 | Q9NQG5 | 0.00488782 | 0.220097415 | 5.971577644 | RPRD1B | yes | T.B.D. | Up H2 | Up H3 | Up H3 | T.B.D. |
| 35 | Q8NHW5 | 0.005050767 | 0.220097415 | 5.930479527 | RPLP0P6 | yes | T.B.D. | Up H2 | Up H3 | Up H3 | T.B.D. |
| 36 | Q6UXG3 | 0.005269477 | 0.220097415 | 5.877438545 | CD300LG | yes | T.B.D. | Down H2 | Up H3 | Up H3 | T.B.D. |
| 37 | Q9Y2Z0 | 0.005865416 | 0.220097415 | 5.743804455 | SUGT1 | yes | T.B.D. | Up H2 | Up H3 | Up H3 | T.B.D. |
| 38 | A5A3E0 | 0.00721476 | 0.220097415 | 5.487272739 | POTEF | yes | T.B.D. | Up H2 | Up H3 | Down H3 | T.B.D. |
| 39 | Q15046 | 0.010250654 | 0.257701434 | 5.057272911 | KARS | yes | T.B.D. | Up H2 | Up H3 | Up H3 | T.B.D. |
| 40 | O75306 | 0.010613548 | 0.261592736 | 5.015027523 | NDUFS2 | yes | T.B.D. | Up H2 | Up H3 | Down H3 | T.B.D. |
| 41 | P55795 | 0.01129597 | 0.267906306 | 4.939514637 | HNRNPH2 | yes | T.B.D. | Up H2 | Up H3 | Up H3 | T.B.D. |
| 42 | O43852-2 | 0.01160955 | 0.26933089 | 4.906396389 | CALU | yes | T.B.D. | Up H2 | Up H3 | Up H3 | T.B.D. |
| 43 | P55884 | 0.012088996 | 0.26933089 | 4.857522011 | EIF3B | yes | T.B.D. | Down H2 | Up H3 | Up H3 | T.B.D. |
| 44 | Q9BWU0 | 0.012345209 | 0.26933089 | 4.8322258 | SLC4A1AP | yes | T.B.D. | Up H2 | Up H3 | Up H3 | T.B.D. |
| 45 | P46782 | 0.01242736 | 0.26933089 | 4.824230671 | RPS5 | yes | T.B.D. | Up H2 | Up H3 | Down H3 | T.B.D. |
| 46 | Q6UX71 | 0.012772194 | 0.272112683 | 4.791261196 | PLXDC2 | yes | T.B.D. | Up H2 | Down H3 | Down H3 | T.B.D. |
| 47 | Q6UXG2 | 0.01324416 | 0.277465145 | 4.747610569 | KIAA1324 | yes | T.B.D. | Up H2 | Up H3 | Up H3 | T.B.D. |
| 48 | P22897 | 0.014702935 | 0.299546134 | 4.622289181 | MRC1 | yes | T.B.D. | Up H2 | Up H3 | Up H3 | T.B.D. |
| 49 | Q96P16 | 0.014796831 | 0.299546134 | 4.614672184 | RPRD1A | yes | T.B.D. | Down H2 | Up H3 | Up H3 | T.B.D. |
| 50 | P34897 | 0.015248769 | 0.299546134 | 4.578701496 | SHMT2 | yes | T.B.D. | Up H2 | Up H3 | Up H3 | T.B.D. |
| 51 | P50991 | 0.015384493 | 0.299546134 | 4.568115711 | CCT4 | yes | T.B.D. | Up H2 | Up H3 | Up H3 | T.B.D. |
| 52 | Q53HC9 | 0.016831151 | 0.299546134 | 4.460979462 | TSSC1 | yes | T.B.D. | Down H2 | Up H3 | Up H3 | T.B.D. |
| 53 | Q9UKT9 | 0.016953656 | 0.299546134 | 4.452352047 | IKZF3 | yes | T.B.D. | Up H2 | Up H3 | Up H3 | T.B.D. |
| 54 | Q7Z7E8 | 0.017060978 | 0.299546134 | 4.444847107 | UBE2Q1 | yes | T.B.D. | Up H2 | Up H3 | Up H3 | T.B.D. |
| 55 | O00233 | 0.017963635 | 0.305783473 | 4.383607388 | PSMD9 | yes | T.B.D. | Up H2 | Up H3 | Up H3 | T.B.D. |
| 56 | P08621 | 0.018244837 | 0.305783473 | 4.365183353 | SNRNP70 | yes | T.B.D. | Up H2 | Up H3 | Up H3 | T.B.D. |
| 57 | P11234 | 0.018597743 | 0.307596876 | 4.342475891 | RALB | yes | T.B.D. | Up H2 | Up H3 | Down H3 | T.B.D. |
| 58 | Q99798 | 0.018888468 | 0.307987276 | 4.324104309 | ACO2 | yes | T.B.D. | Down H2 | Up H3 | Up H3 | T.B.D. |
| 59 | Q92614 | 0.019111382 | 0.307987276 | 4.310216427 | MYO18A | yes | T.B.D. | Up H2 | Up H3 | Down H3 | T.B.D. |
| 60 | P47897 | 0.019857326 | 0.308754485 | 4.264941692 | QARS | yes | T.B.D. | Up H2 | Up H3 | Up H3 | T.B.D. |

| | D) Optional biomarkers (H-grade and DMFS) | | | | | | | Up- or down-regulation in individual comparisons | | | |
|---|---|---|---|---|---|---|---|---|---|---|---|
| | Prot acc. | p-Value | q-Value | F-statistic | Name | H-grade | DMSF | H1 vs H2 | H1 vs H3 | H2 vs H3 | DMFS |
| 61 | Q13310 | 0.000177815 | 0.055878409 | 10.43467236 | PABPC4 | yes | yes | Up H2 | Up H3 | Down H3 | Up H3 |
| 62 | O95969 | 0.000669988 | 0.123381185 | 8.572206497 | SCGB1D2 | yes | yes | Down H2 | Down H3 | Down H3 | Down H3 |
| 63 | Q01813 | 0.00080514 | 0.123381185 | 8.322399139 | PFKP | yes | yes | Up H2 | Up H3 | Up H3 | Up H3 |
| 64 | P08195 | 0.001042488 | 0.123381185 | 7.974472523 | SLC3A2 | yes | yes | Up H2 | Up H3 | Up H3 | Up H3 |
| 65 | Q9BXN1 | 0.002542201 | 0.194665471 | 6.802918911 | ASPN | yes | yes | Down H2 | Down H3 | Down H3 | Down H3 |
| 66 | P28907 | 0.003943867 | 0.215540903 | 6.241922855 | CD38 | yes | yes | Up H2 | Up H3 | Up H3 | Up H3 |
| 67 | Q9NR99 | 0.00573962 | 0.220097415 | 5.770796299 | MXRA5 | yes | yes | Down H2 | Down H3 | Down H3 | Down H3 |
| 68 | P06493 | 0.006362452 | 0.220097415 | 5.642757893 | CDK1 | yes | yes | Up H2 | Up H3 | Up H3 | Up H3 |
| 69 | O76061 | 0.006825774 | 0.220097415 | 5.555717945 | STC2 | yes | yes | Down H2 | Down H3 | Down H3 | Down H3 |
| 70 | P53634 | 0.007255011 | 0.220097415 | 5.480411053 | CTSC | yes | yes | Up H2 | Up H3 | Down H3 | Up H3 |

| | E) Optional biomarkers (H-grade) | | | | | | | Up- or down regulation in individual comparisons | | | |
|---|---|---|---|---|---|---|---|---|---|---|---|
| | Prot acc. | p-Value | q-Value | F-statistic | Name | H-grade | DMSF | H1 vs H2 | H1 vs H3 | H2 vs H3 | DMSF* |
| 71 | Q9Y2X3 | 0.0078144 | 0.220097415 | 5.388957977 | NOP58 | yes | T.B.D. | Up H2 | Up H3 | Up H3 | T.B.D. |
| 72 | P00558 | 0.011192822 | 0.267906306 | 4.950618267 | PGK1 | yes | T.B.D. | Up H2 | Up H3 | Up H3 | T.B.D. |
| 73 | Q00688 | 0.011943688 | 0.26933089 | 4.872117996 | FKBP3 | yes | T.B.D. | Up H2 | Up H3 | Up H3 | T.B.D. |
| 74 | P21266 | 0.016301906 | 0.299546134 | 4.499019623 | GSTM3 | yes | T.B.D. | Down H2 | Down H3 | Down H3 | T.B.D. |
| 75 | Q9NZT1 | 0.016391 | 0.299546134 | 4.492526531 | CALML5 | yes | T.B.D. | Up H2 | Up H3 | Up H3 | T.B.D. |
| 76 | P29590 | 0.016807432 | 0.299546134 | 4.462657452 | PML | yes | T.B.D. | Up H2 | Up H3 | Up H3 | T.B.D. |
| 77 | O75173 | 0.016811191 | 0.299546134 | 4.462391376 | ADAMTS4 | yes | T.B.D. | Down H2 | Down H3 | Down H3 | T.B.D. |
| 78 | P07996 | 0.017157774 | 0.299546134 | 4.438120365 | THBS1 | yes | T.B.D. | Down H2 | Down H3 | Down H3 | T.B.D. |
| 79 | P02751 | 0.018241382 | 0.305783473 | 4.365407944 | FN1 | yes | T.B.D. | Down H2 | Down H3 | Down H3 | T.B.D. |

*based on median H1/H3 ratio
T.B.D. = to be determined

TABLE 2
RECOMMENDED NAMES OF BIOMARKERS FOR DETERMINING
A BREAST CANCER-ASSOCIATED DISEASE STATE

| Prot acc. | Name | Recommended name |
|---|---|---|
| Q9HCB6 | SPON1 | Spondin-1 |
| O60938 | KERA | Keratocan |
| P02743 | APCS | Serum amyloid P-component |
| Q7Z5L7 | PODN | Podocan |
| O75348 | ATP6V1G1 | V-type proton ATPase subunit G 1 |
| Q71UM5 | RPS27L | 40S ribosomal protein S27-like |
| Q14195 | DPYSL3 | Dihydropyrimidinase-related protein 3 |
| Q9BS26 | ERP44 | Endoplasmic reticulum resident protein 44 |
| Q13905 | RAPGEF1 | Rap guanine nucleotide exchange factor 1 |
| P53396 | ACLY | ATP-citrate synthase |
| P23946 | CMA1 | Chymase |
| P25205 | MCM3 | DNA replication licensing factor MCM3 |
| Q9UKU9 | ANGPTL2 | Angiopoietin-related protein 2 |
| Q8IUX7 | AEBP1 | Adipocyte enhancer-binding protein 1 |
| Q15819 | UBE2V2 | Ubiquitin-conjugating enzyme E2 variant 2 |
| Q6P0N0 | MIS18BP1 | Mis18-binding protein 1 |
| Q9UBD9 | CLCF1 | Cardiotrophin-like cytokine factor 1 |
| P80404 | ABAT | 4-aminobutyrate aminotransferase, |
| | | mitochondrial |
| P05141 | SLC25A5 | ADP/ATP translocase 2 |
| Q9NQG5 | RPRD1B | Regulation of nuclear pre-mRNA domain- |
| | | containing protein 1B |
| P31948 | STIP1 | Stress-induced-phosphoprotein 1 |
| Q8NHW5 | RPLP0P6 | 60S acidic ribosomal protein P0-like |
| Q6UXG3 | CD300LG | CMRF35-like molecule 9 |
| Q9NRN5 | OLFML3 | Olfactomedin-like protein 3 |
| Q9Y2Z0 | SUGT1 | Suppressor of G2 allele of SKP1 homolog |
| P09693 | CD3G | T-cell surface glycoprotein CD3 gamma chain |
| P33993 | MCM7 | DNA replication licensing factor MCM7 |
| Q02978 | SLC25A11 | Mitochondrial 2-oxoglutarate/malate carrier |
| | | protein |
| O00567 | NOP56 | Nucleolar protein 56 |
| O43159 | RRP8 | Ribosomal RNA-processing protein 8 |
| A5A3E0 | POTEF | POTE ankyrin domain family member F |
| Q9NWH9 | SLTM | SAFB-like transcription modulator |
| Q15631 | TSN | Translin |
| Q13011 | ECH1 | Delta(3,5)-Delta(2,4)-dienoyl-CoA isomerase, |
| | | mitochondrial |
| P51888 | PRELP | Prolargin |
| P49591 | SARS | Serine--tRNA ligase, cytoplasmic |
| P62851 | RPS25 | 40S ribosomal protein S25 |
| Q9BSJ8 | ESYT1 | Extended synaptotagmin-1 |
| Q15046 | KARS | Lysine--tRNA ligase |
| O75306 | NDUFS2 | NADH dehydrogenase [ubiquinone] iron-sulfur |
| | | protein 2, mitochondrial |
| P55795 | HNRNPH2 | Heterogeneous nuclear ribonucleoprotein H2 |
| O43852-2 | CALU | Calumenin |
| P55884 | EIF3B | Eukaryotic translation initiation factor 3 |
| | | subunit B |
| Q9BWU0 | SLC4A1AP | Kanadaptin |
| P46782 | RPS5 | 40S ribosomal protein S5 |
| Q6UX71 | PLXDC2 | Plexin domain-containing protein 2 |
| Q6UXG2 | KIAA1324 | UPF0577 protein KIAA1324 |
| P22897 | MRC1 | Macrophage mannose receptor 1 |
| Q96P16 | RPRD1A | Regulation of nuclear pre-mRNA domain- |
| | | containing protein 1A |
| P34897 | SHMT2 | Serine hydroxymethyltransferase, |
| | | mitochondrial |
| P50991 | CCT4 | T-complex protein 1 subunit delta |
| Q53HC9 | TSSC1 | Protein TSSC1 |
| Q9UKT9 | IKZF3 | Zinc finger protein Aiolos |
| Q7Z7E8 | UBE2Q1 | Ubiquitin-conjugating enzyme E2 Q1 |
| O00233 | PSMD9 | 26S proteasome non-ATPase regulatory |
| | | subunit 9 |
| P08621 | SNRNP70 | U1 small nuclear ribonucleoprotein 70 kDa |
| P11234 | RALB | Ras-related protein Ral-B |
| Q99798 | ACO2 | Aconitate hydratase, mitochondrial |
| Q92614 | MYO18A | Unconventional myosin-XVIIIa |
| P47897 | QARS | Glutamine--tRNA ligase |
| Q13310 | PABPC4 | Polyadenylate-binding protein 4 |
| O95969 | SCGB1D2 | Secretoglobin family 1D member 2 |
| Q01813 | PFKP | 6-phosphofructokinase type C |
| P08195 | SLC3A2 | 4F2 cell-surface antigen heavy chain |
| Q9BXN1 | ASPN | Asporin |
| P28907 | CD38 | ADP-ribosyl cyclase 1 |
| Q9NR99 | MXRA5 | Matrix-remodeling-associated protein 5 |
| P06493 | CDK1 | Cyclin-dependent kinase 1 |
| O76061 | STC2 | Stanniocalcin-2 |
| P53634 | CTSC | Dipeptidyl peptidase 1 |
| Q9Y2X3 | NOP58 | Nucleolar protein 58 |
| P00558 | PGK1 | Phosphoglycerate kinase 1 |
| Q00688 | FKBP3 | Peptidyl-prolyl cis-trans isomerase FKBP3 |
| P21266 | GSTM3 | Glutathione S-transferase Mu 3 |
| Q9NZT1 | CALML5 | Calmodulin-like protein 5 |
| P29590 | PML | Protein PML |
| O75173 | ADAMTS4 | A disintegrin and metalloproteinase with |
| | | thrombospondin motifs 4 |
| P07996 | THBS1 | Thrombospondin-1 |
| P02751 | FN1 | Fibronectin |

TABLE 3
ROC AUC VALUES FOR EXEMPLARY
BIOMARKER COMBINATIONS

| | ROC AUC value | | |
|---|---|---|---|
| Biomarker signature | H1 vs H3 | H2 vs H3 | H1 vs H2 |
| Core-1 | 0.94 | 0.82 | 0.69 |
| Core-2 | 0.82 | 0.82 | 0.56 |
| Core-1 + core-2 | 0.94 | 0.83 | 0.50 |
| Core-1 + core-2 + (marker 3-12) | 0.90 | 0.85 | 0.69 |
| Core-1 + core-2 + (marker 3-22) | 0.88 | 0.76 | 0.88 |
| Core-1 + core-2 + (marker 3-32) | 0.81 | 0.73 | 0.94 |
| Core-1 + core-2 + (marker 3-42) | 0.81 | 0.74 | 0.87 |
| Core-1 + core-2 + (marker 3-52) | 0.80 | 0.77 | 0.76 |
| Core-1 + core-2 + (marker 3-60) | 0.80 | 0.77 | 0.76 |
| Core-1 + core-2 + (marker 3-62) | 0.93 | 0.86 | 0.76 |
| Core-1 + core-2 + (marker 3-72) | 0.94 | 0.83 | 0.82 |
| Core-1 + core-2 + (marker 3-79) | 0.94 | 0.79 | 0.86 |

TABLE 4
HISTOLOGICAL GRADE SVM SCRIPT

filnamn<−“Input. txt”

# 1.1 Change FILNAME to datafile ------------------

```
---
# Läser in och logaritmerar datan
rawfile <− read.delim(filnamn)
samplenames <− as.character(rawfile[,1])
Diagnosis <− rawfile[,2]
Morphology<− rawfile[,3]
Treatment<−rawfile[,4]
data <− t(rawfile[,−c(1:4)])
ProteinNames <− read.delim(filnamn,header=FALSE)
ProteinNames <− as.character(as.matrix(ProteinNames)[1,])
ProteinNames <− ProteinNames[−(1:4)]
rownames(data) <− ProteinNames
colnames(data) <− samplenames
logdata <− log(data)/log(2)
# Tar reda p\dot{a} vilka gruppjämförelser som ska göras
PairWiseGroups <−
as.matrix(read.delim(“Comparison_to_do.txt”,header=FALSE)) # 1.2
Change filname and use criteria file -----
# Definierar Wilcoxontestet
wilcoxtest <− function(prot,subset1,subset2){
  res <− wilcox.test(prot[subset1],prot[subset2])
  res$p.value
}
# Definierar foldchange
foldchange <− function(prot,subset1,subset2) {
```

2\hat{ }(mean(prot[subset1]) − mean(prot[subset2]))

}
# Definierar q-värdesberäkningen
BenjaminiHochberg <− function(pvalues){

```
  # This function takes a vector of p-values as input and outputs
  # their q-values. No reordering of the values is performed
  NAindices <− is.na(pvalues)
  Aindices <− !NAindices
  Apvalues <− pvalues[Aindices]
  N <− length(Apvalues)
  orderedindices <− order(Apvalues)
  OrdValues <− Apvalues[orderedindices]
  CorrectedValues <− OrdValues * N /(1:N)
  MinValues <− CorrectedValues
  for (i in 1:N) {MinValues[i] <− min(CorrectedValues[i:N])}
  Aqvalues <− numeric(N)
  Aqvalues[orderedindices] <− MinValues
  Qvalues <− pvalues
  Qvalues[Aindices] <− Aqvalues
  return(Qvalues)
```

}
# Laddar in tv\dot{a} bibliotek
library(MASS)
library(gplots)
# Definierar farger till heatmapen
redgreen <− function(n)

{

c(

hsv(h=0/6, v=c( rep( seq(1,0.3,length=5) , c(13,10,8,6,4) ) ,

0 ) ) ,

hsv(h=2/6, v=c( 0 , rep( seq(0.3,1,length=5) ,

c(3,5,7,9,11) ) ) )

)

}

```
pal <− rev(redgreen(100));
#Laddar in fler bibliotek och funktioner
library(e1071)
source(“NaiveBayesian”)
#Definierar SVM med Leave One Out
svmLOOvalues <− function(data , fac){
  n1 <− sum(fac==levels(fac)[1])
  n2 <− sum(fac==levels(fac)[2])
  nsamples <− n1+n2
  ngenes <− nrow(data)
  SampleInformation <− paste(levels(fac)[1],“ ”,n1,“ ,
”,levels(fac)[2],“ ”,n2,sep=“”)
  res <− numeric(nsamples)
  sign <− numeric(nsamples)
  for (i in 1:nsamples){
```

svmtrain <− svm(t(data[,−i]) , fac[−i] , kernel=“linear” )
pred <− predict(svmtrain , t(data[,i]) , decision.values=TRUE)
res[i] <− as.numeric(attributes(pred)$decision.values)
facnames <− colnames(attributes(pred)$decision.values)[1]
if (facnames ==

paste(levels(fac)[1],“/”,levels(fac)[2],sep=“”)) {sign[i] <− 1}

if (facnames ==

```
paste(levels(fac)[2],“/”,levels(fac)[1],sep=“”)){sign[i] <− −1}
  }
  if (length(unique(sign)) >1){print(“error”)}
  res <− sign * res
  names <− colnames(data , do.NULL=FALSE)
  orden <− order(res , decreasing=TRUE)
  Samples <− data.frame(names[orden],res[orden],fac[orden])
  ROCdata <− myROC(res,fac)
  SenSpe <− SensitivitySpecificity(res,fac)
return(list(SampleInformation=SampleInformation,ROCarea=ROCdata[1],p.
value=ROCdata[2],SenSpe <− SenSpe, samples=Samples))
}
# Definierar hur analysen ska köras om man INTE ANVÄNDER
apriorianalyter
Analysera<− function (group1 ,group2){
```

outputfiletxt <− paste(group1,“ versus ”,group2,“.txt” ,sep=“”)
outputfilepdf <− paste (group1,“ versus ”, group2,“.pdf” ,sep=“”)
#outputfilejpeg <− paste(group1,“ versus ”,group2,“.jpg” ,sep=“”)
subset1 <− is.element (Diagnosis , strsplit (group1,“,”)[[1]])
subset2 <− is.element(Diagnosis , strsplit(group2,“,”)[[1]])
wilcoxpvalues <− apply(logdata , 1 , wilcoxtest , subset1 ,

subset2)

foldchange <− apply(logdata , 1 , foldchange , subset1 , subset2)
QvaluesAll <− BenjaminiHochberg(wilcoxpvalues)
HugeTable <−

cbind(ProteinNames,foldchange,wilcoxpvalues,QvaluesAll)

write.table(HugeTable, file=outputfiletxt , quote=FALSE,

sep=“\t”,row.names=FALSE)

color <− rep(‘black’ , length(subset1))
color[subset1] <− ‘red’
color[subset2] <− ‘blue’
pdf(outputfilepdf)
#jpeg(outputfilejpeg, quality=100, width=600, height=600)
Sam <− sammon(dist(t(logdata[,subset1|subset2])) , k=2)
plot(Sam$points , type=“n” , xlab = NA , ylab=NA, main=“All

proteins” ,asp=1)

text(Sam$point , labels = colnames(logdata[,subset1|subset2]),

col=color[subset1|subset2])

heatmap.2(logdata[,subset1|subset2] , labRow = row.names(logdata) ,

trace=“none” , labCol =“” , ColSideColors=
color[subset1|subset2],col=pal , na.color= “grey”, key=FALSE , symkey
=FALSE , tracecol = “black” , main =“” , dendrogram= ‘both’ , scale
=“row” ,cexRow=0.2)

svmfac <−

factor(rep(‘rest’,ncol(logdata)),levels=c(group1,group2,‘rest’))

svmfac[subset1] <− group1
svmfac[subset2] <− group2
svmResAll <− svmLOOvalues(logdata[,subset1|subset2] ,

factor(as.character(svmfac[subset1|subset2]), levels=c(group1, group2))
)

ROCplot(svmResAll , sensspecnumber=4)
# N <− length(ProteinNames)
# par(mfrow=c(1,2))
# for (k in 1:N){
# boxplot(data[k,subset1],data[k,subset2], names=c(group1, group2),

main=c(ProteinNames[k],“ test”))

# }
write(“” , file=outputfiletxt , append=TRUE)
write(“All proteins” , file=outputfiletxt , append=TRUE)
write(“” , file=outputfiletxt , append=TRUE)
for (i in 1:5){write.table(svmResAll[[i]], file=outputfiletxt ,

append=TRUE, sep=“\t” , quote=FALSE)

write( “” , file=outputfiletxt , append=TRUE)

}
dev.off( )

}

| | |
|---|---|
| Analysera(“X”,”Y”) | # 1.3 Select comparisons to do--------------- |

------

Supplemental Table 1

| | | | Tumor | ER/PgR/ | | |
|---|---|---|---|---|---|---|
| Sample | Hist. | | size | HER2/ | | Nr of |
| ID | Grade | Age | (mm) | ki67_gt_25 | Lymph_pos | Lymph pos. |
| 6616 | 1 | 37, 62 | 22 | +/+/−/− | yes | 1 |
| 6617 | 1 | 66, 30 | 20 | +/+/−/na | yes | 5 |
| 7149 | 1 | 74, 49 | 31 | +/+/−/− | no | 0 |
| 7454 | 1 | 47, 82 | 22 | +/+/−/− | yes | 5 |
| 7940 | 1 | 53, 94 | 30 | +/+/−/− | no | 0 |
| 8415 | 1 | 66, 61 | 31 | +/+/−/− | yes | 4 |
| 9317 | 1 | 47, 42 | 18 | +/+/−/− | no | 0 |
| 9795 | 1 | 43, 26 | 15 | +/+/−/− | yes | 2 |
| 10524 | 1 | 64, 34 | 30 | +/+/−/− | no | 0 |
| 4404 | 2 | 49, 92 | 25 | +/+/−/+ | yes | 1 |
| 5614 | 2 | 45, 48 | 37 | +/+/−/− | yes | 8 |
| 5096 | 2 | 37, 35 | 6 | +/+/−/+ | yes | 8 |
| 5572 | 2 | 43, 55 | 18 | −/−/−/− | yes | 2 |
| 6096 | 2 | 36, 92 | 12 | +/+/−/− | yes | 1 |
| 6627 | 2 | 43, 63 | 15 | +/+/−/− | yes | 2 |
| 7015 | 2 | 46, 77 | 22 | +/+/−/na | yes | 1 |
| 7267 | 2 | 48, 39 | 22 | +/−/−/− | yes | 1 |
| 7296 | 2 | 46, 38 | 14 | +/+/−/na | yes | 4 |
| 8173 | 2 | 47, 03 | 25 | +/+/−/na | yes | 10 |
| 9257 | 2 | 43, 78 | 7 | +/+/−/+ | no | 0 |
| 9340 | 2 | 52, 10 | 29 | +/+/−/− | no | 0 |
| 5402 | 2 | 44, 26 | 50 | −/+/−/− | yes | 5 |
| 6514 | 2 | 49, 18 | 30 | −/−/na/na | no | 0 |
| 7424 | 2 | 47, 98 | 25 | +/−/−/+ | yes | 1 |
| 8278 | 2 | 47, 54 | 10 | +/+/−/− | yes | 1 |
| 8504 | 2 | 49, 66 | 25 | +/+/−/− | yes | 1 |
| 5706 | 3 | 41, 19 | 50 | −/−/−/+ | yes | 5 |
| 4239 | 3 | 40, 66 | 33 | −/−/−/+ | no | 0 |
| 5744 | 3 | 44, 04 | 21 | +/+/na/− | no | 0 |
| 5811 | 3 | 49, 75 | 45 | −/−/−/+ | yes | 1 |
| 5997 | 3 | 46, 37 | 20 | −/−/−/na | no | 0 |
| 6009 | 3 | 49, 57 | 20 | +/+/−/− | no | 0 |
| 6029 | 3 | 42, 80 | 25 | −/−/na/+ | yes | 4 |
| 6158 | 3 | 55, 81 | 20 | +/+/+/+ | yes | 2 |
| 6191 | 3 | 45, 04 | 25 | −/−/−/− | no | 0 |
| 6276 | 3 | 52, 30 | 32 | −/−/+/+ | yes | 6 |
| 4723 | 3 | 48, 89 | 40 | −/−/+/+ | yes | 3 |
| 5198 | 3 | 46, 66 | 32 | −/+/+/na | yes | 4 |
| 5203 | 3 | 33, 22 | 30 | −/−/−/− | yes | 1 |
| 5634 | 3 | 44, 33 | 25 | +/+/−/na | yes | 2 |
| 5996 | 3 | 50, 94 | 22 | −/−/+/− | yes | 2 |
| 6013 | 3 | 41, 60 | 50 | +/+/−/na | yes | 3 |
| 6176 | 3 | 50, 62 | 35 | +/+/−/+ | no | 0 |
| 6503 | 3 | 43, 39 | 28 | −/−/−/+ | no | 0 |
| 6877 | 3 | 34, 39 | 27 | +/+/−/+ | yes | 8 |
| 7694 | 3 | 47, 66 | 18 | −/−/−/+ | yes | 1 |
| 7722 | 3 | 46, 61 | 27 | +/+/na/− | no | 0 |
| 8613 | 3 | 44, 04 | 35 | +/+/−/− | yes | 6 |
| 9322 | 3 | 50, 33 | 30 | −/−/−/+ | no | 0 |
| 9460 | 3 | 49, 01 | 17 | +/+/−/+ | no | 0 |
| 5784 | na | na | na | na/na/na/na | na | na |
| 4917 | na | na | na | na/na/na/na | na | na |

na = not available

SUPPLEMENTAL TABLE 2

| | | Affinity (K_{D}) | |
|---|---|---|---|
| CIMS antibody* | Selection peptide | (μM) | Mix |
| CIMS-33-3C-A09 | Biotin-SGSGLSADHR | 1.6 | 1 |
| CIMS-33-3D-F06 | Biotin-SGSGLSADHR | 5.1 | 1 |
| CIMS-17-C08 | Biotin-SGSGSSAYSR | 0.2 | 2 |
| CIMS-17-E02 | Biotin-SGSGSSAYSR | 0.4 | 2 |
| CIMS-15-A06 | Biotin-SGSGLTEFAK | 2.2 | 3 |
| CIMS-34-3A-D10 | Biotin-SGSGSEAHLR | 2.5 | 3 |
| CIMS-1-B03 | Biotin-SGSGEDFR | 3.5 | 4 |
| CIMS-31-001-D01 | Biotin-SGSGLNVWGK | NA | 4 |
| CIMS-32-3A-G03 | Biotin-SGSGQEASFK | 11.5 | 4 |

*For details regarding binder characteristics see Olsson et al. (2011) MCP M110.003962.

1. A method for determining a breast cancer-associated disease state comprising the steps of:
a) providing a sample to be tested; and
b) determining a biomarker signature of the test sample by measuring the presence and/or amount in the test sample of one or more biomarker selected from the group defined in Table 1A, Table 1B and/or Table 1C;
wherein the presence and/or amount in the test sample of the one or more biomarker selected from the group defined in Table 1A, Table 1B and/or Table 1C is indicative of the breast cancer-associated disease state.

2. The method according to claim 1 wherein the breast cancer associated disease state is the histological grade and/or the metastasis-free survival time.

3. The method according to claim 1 wherein the breast cancer-associated disease state is the histological grade of breast cancer cells.

4. The method according to claim 3 wherein the method further comprises the steps of:
c) providing one or more control sample comprising or consisting of histological grade 1 breast cancer cells, histological grade 2 breast cancer cells and/or histological grade 3 breast cancer cells; and
d) determining a biomarker signature of the control sample(s) by measuring the presence and/or amount in the control sample(s) of the one or more biomarker measured in step (b);
wherein the presence of breast cancer cells is identified in the event that the presence and/or amount in the test sample of the one or more biomarker measured in step (b):
i) corresponds to the presence and/or amount in a control sample comprising or consisting breast cancer cells of a first histological grade (where present);
ii) is different to the presence and/or amount in a control sample comprising or consisting breast cancer cells of a second histological grade (where present); and/or
iii) is different to the presence and/or amount in a control sample comprising or consisting breast cancer cells of a third histological grade (where present).

5. The method according to claim 4 wherein each control sample comprises or consists of a single histological grade of breast cancer cells.

6. The method according to claim 4 wherein step (c) comprises or consists of:
i) providing one or more control sample comprising or consisting of histological grade 1 breast cancer cells; providing one or more control sample comprising or consisting of histological grade 2 breast cancer cells; and providing one or more control sample comprising or consisting of histological grade 3 breast cancer cells;
ii) providing one or more control sample comprising or consisting of histological grade 1 breast cancer cells; and providing one or more control sample comprising or consisting of histological grade 2 breast cancer cells;
iii) providing one or more control sample comprising or consisting of histological grade 1 breast cancer cells; and providing one or more control sample comprising or consisting of histological grade 3 breast cancer cells;
iv) providing one or more control sample comprising or consisting of histological grade 2 breast cancer cells; and providing one or more control sample comprising or consisting of histological grade 3 breast cancer cells;
v) providing one or more control sample comprising or consisting of histological grade 1 breast cancer cells;
vi) providing one or more control sample comprising or consisting of histological grade 2 breast cancer cells; or
vii) providing one or more control sample comprising or consisting of histological grade 3 breast cancer cells.

7. The method according to claim 1 wherein the breast cancer-associated disease state is the metastasis-free survival time of an individual.

8. The method according to claim 7 wherein the method further comprises the steps of:
c) providing one or more first control sample comprising or consisting of breast cancer cells from an individual with less than 10 years metastasis-free survival; and/or one or more second control sample comprising or consisting of breast cancer cells from an individual with 10 or more years metastasis free survival; and
d) determining a biomarker signature of the control sample(s) by measuring the presence and/or amount in the control sample(s) of the one or more biomarker measured in step (b);
wherein the metastasis-free survival time of an individual is identified as less than 10 years in the event that the presence and/or amount of the one or more biomarker measured in step (b) corresponds to the presence and/or amount of the first control sample (where present) and/or is different to the presence and/or amount of the second control sample (where present);
and wherein the metastasis-free survival time of an individual is identified as more than 10 years in the event that the presence and/or amount of the one or more biomarker measured in step (b) is different to the presence and/or amount of the first control sample (where present) and/or corresponds to the presence and/or amount of the second control sample (where present).

9. The method according to claim 8 wherein the one or more first and/or second control sample is of the same histological grade as the sample to be tested.

10. A method according to claim 3 wherein step (b) comprises or consists of measuring the presence and/or amount in the test sample of:
one or more biomarkers selected from the group defined in Table 1A, for example at least 2, biomarkers selected from the group defined in Table 1A;
one or more biomarkers selected from the group defined in Table 1B, for example at least 2, 3, 4, 5, 6, 7, 8, 9, 10, 11, 12, 13, 14, 15, 16, 17, 18, 19, 20, 21, 22, 23, 24, 25, 26, 27, 28, 29 or at least 30 biomarkers selected from the group defined in Table 1B;
one or more biomarkers selected from the group defined in Table 1C, for example at least 2, 3, 4, 5, 6, 7, 8, 9, 10, 11, 12, 13, 14, 15, 16, 17, 18, 19, 20, 21, 22, 23, 24, 25, 26, 27 or at least 28 biomarkers selected from the group defined in Table 1C;
one or more biomarkers selected from the group defined in Table 1D, for example at least 2, 3, 4, 5, 6, 7, 8, 9 or at least 10 biomarkers selected from the group defined in Table 1D; and/or
one or more biomarkers selected from the group defined in Table 1E, for example at least 2, 3, 4, 5, 6, 7, 8 or at least 9 biomarkers selected from the group defined in Table 1E.

11-14. (canceled)

15. A method according to claim 3 wherein step (b) comprises or consists of measuring the presence and/or amount in the test sample of all of the biomarkers defined in Table 1.

16-23. (canceled)

24. The method according to claim 1 wherein step (b) comprises measuring the expression of a nucleic acid molecule encoding the one or more biomarker(s).

25-28. (canceled)

29. The method according to claim 1 wherein measuring the expression of the one or more biomarker(s) in step (b) is performed using one or more binding moieties, each capable of binding selectively to a nucleic acid molecule encoding one of the biomarkers identified in Table 1.

30-35. (canceled)

36. The method according to claim 1 wherein step (b) comprises measuring the expression of the protein or polypeptide of the one or more biomarker(s).

37. The method according to claim 36 wherein measuring the expression of the one or more biomarker(s) in step (b) is performed using one or more binding moieties each capable of binding selectively to one of the biomarkers identified in Table 1.

38-48. (canceled)

49. The method according to claim 1 wherein the predicative accuracy of the method, as determined by an ROC AUC value, is at least 0.50, for example at least 0.55, 0.60, 0.65, 0.70, 0.75, 0.80, 0.85, 0.90, 0.95, 0.96, 0.97, 0.98 or at least 0.99.

50. (canceled)

51. The method according to claim 1 wherein step (b) is performed using an array.

52-54. (canceled)

55. An array for determining a breast cancer-associated disease state, the array comprising one or more first binding agents as defined in claim 29.

56-62. (canceled)

63. Use of one or more biomarkers selected from the group defined in Table 1A, Table 1B and/or Table 1C for determining a breast cancer-associated disease state.

64. (canceled)

65. An analytical kit for determining a breast cancer-associated disease state comprising:
A) an array according to claim 55; and
B) instructions for performing the method (optional).

66-68. (canceled)
